(19) United States
(12) Patent Application Publication
(10) Pub. No.: US 2026/0183383 A1
(43) Pub. Date: Jul. 2, 2026

(54) SARS-COV-2 IMMUNOGENIC COMPOSITIONS AND METHODS

(71) Applicant: CAPRICOR, INC., San Diego, CA (US)

(72) Inventors: Minghao SUN; Kristi ELLIOTT; Mafalda CACCIOTTOLO; Yujia LI; Justin NICE; Michael LECLAIRE; Li-En HSIEH

(21) Appl. No.: 19/106,167

(22) PCT Filed: Aug. 24, 2023
(86) PCT No.: PCT/US2023/072876

§ 371 Date: Feb. 24, 2025

(30) Foreign Application Priority Data
Apr. 10, 2022 (US) 63413193
Aug. 24, 2022 (US) 63373418
Sep. 28, 2022 (US) 63411090
Jan. 8, 2023 (US) 63437710
Mar. 31, 2023 (US) 63456380

(51) Int. Cl. A61K 39/215; A61K 39/00; A61P 37/04; C07K 14/005; C07K 14/705

(52) U.S. Cl. CPC A61K 39/215; A61P 37/04; C07K 14/005; C07K 14/70596; A61K 2039/55555; C12N 2770/20022; C12N 2770/20034

(57) ABSTRACT

The present disclosure relates to compositions and methods for vaccinating a subject against multiple SARS-CoV-2 variants that involves the making and delivery of extracellular vesicles expressing on their surface engineered spike protein and/or engineered nucleocapsid protein to the subject. The present invention also relates to compositions and methods for the design, preparation, manufacture, formulation, and/or use of spike-display and nucleocapsid-display vesicular vaccines designed to elicit strong humoral and cellular immune responses against multiple SARS-CoV-2 variants.

### REFERENCE TO RELATED APPLICATIONS

This application claims the benefit of the priority dates of U.S. Provisional Application No. 63/373,418, filed Aug. 24, 2022, U.S. Provisional Application No. 63/411,090 filed Sep. 28, 2022, U.S. Provisional Application No. 63/413,193, filed Oct. 4, 2022, U.S. Provisional Application No. 63/437,710, filed Jan. 8, 2023, and U.S. Provisional Application No. 63/456,380, filed Mar. 31, 2023, the contents of which are incorporated herein by reference in their entireties.

### SEQUENCE LISTING

The instant application contains a Sequence Listing which has been submitted electronically in XML file format and is hereby incorporated by reference in its entirety. Said XML copy, created on Aug. 18, 2023, is named 514PCT.xml and is 27,456 bytes in size.

### BACKGROUND

The emergence of severe acute respiratory syndrome coronavirus 2 (SARS-CoV-2) has created an urgent need for vaccine development strategies to produce safe, effective, readily available and accessible vaccines that can be efficiently produced to combat the emergence of evolving SARS-CoV-2 variants. Many vaccines have been developed, which try to achieve immunity to the virus, mainly directing the immune response to surface spike protein, which binds to the host cell receptor angiotensin-converting enzyme 2 (ACE2), mediating viral cell entry and infection. The spike (S) protein is a class I fusion glycoprotein, the major surface protein on the SARS-CoV-2 virus and the primary target for neutralizing antibodies. Spike is also the primary site of mutations identified in SARS-CoV-2 viruses, reducing the efficacy of the immune-response induced by vaccines. Since the beginning of the COVID-19 pandemic, five variants of concern (VOC) and 8 variants of interest (VOI) have been reported, which poses challenges to current vaccines and creating the need for more effective vaccines. Vaccines directed against SARS-CoV-2 nucleocapsid protein, which is an internal soluble coronavirus protein, have heretofore been largely unsuccessful.

Current leading vaccines have employed mRNA-lipid nanoparticle or virus vector technology, with more recent vaccines utilizing recombinant proteins (see, e.g., Krammer, F., SARS-CoV-2 vaccines in development. Nature, 2020. 586(7830): p. 516-527). Compared to the leading mRNA vaccines, recombinant or inactivated protein vaccines are a safe and reliable approach for immunization but generally suffer from weak immunogenicity, thus requiring formulation with an appropriate adjuvant. While all current mRNA vaccine candidates achieve a strong initial immune response to the virus with consequent reduction of hospitalization rates, they all lack long-term protection. It's been reported that antibody levels drastically decline over time and therefore the efficacy to protect against infection drastically declines as well (see, e.g., Xiang, T., et al., Declining Levels of Neutralizing Antibodies Against SARS-CoV-2 in Convalescent COVID-19 Patients One Year Post Symptom Onset. Front Immunol, 2021. 12: p. 708523). Moreover, mRNA vaccines have thus far lacked cross-reactivity against new variants of concern, requiring multiple booster injections to maintain protection against the virus. Thus, there remains a significant medical and public health need for a better, globally deployable vaccine for severe respiratory syndrome coronavirus 2 (SARS-CoV-2) to provide improved, broader, longer lasting neutralization of SARS-CoV-2, a more robust T cell response, and an enhanced safety profile.

Briefly, a problem to which the present invention may be directed includes the production of SARS-CoV-2 variants of concern having more infectivity and less immunogenicity than some variants of lesser concern. This creates an ongoing public health and medical problem in which vaccines are no longer effective against new strains, and new strains that are less immunogenic (long term and short-term immunogenicity).

Disclosed is a solution to this problem, which includes a safe (LNP-free, adjuvant-free) and effective vaccine that confers long term humoral and cell-mediated immunity against multiple, emerging, and recalcitrant variants of SARS-CoV-2, which includes an extracellular vesicle that displays a spike protein from a single variant of SARS-CoV-2, which confers robust humoral and/or cellular immunity against several SARS-CoV-2 variants of concern or interest, and which optionally includes an extracellular vesicle that displays a nucleocapsid protein from a single variant of SARS-CoV-2, which also confers robust humoral and/or cellular immunity against several SARS-CoV-2 variants of concern or interest.

### SUMMARY

In one embodiment, a synthetic fusion protein is provided that contains polypeptide sequences of a SARS-CoV-2 protein fused to polypeptide sequences of an exosomal tetraspanin protein. The fusion protein is designed and made such that a SARS-CoV-2 protein antigen can be expressed on the surface of an exosome to enable the eliciting of an immune response when administered to a subject.

In one aspect, the SARS-CoV-2 protein is a spike protein. In another aspect, the SARS-CoV-2 protein is a nucleocapsid protein. In one aspect, the exosomal tetraspanin protein is a CD9 protein.

In some aspects in which the SARS-CoV-2 protein is a spike protein and the exosomal tetraspanin protein is a CD9 protein, the spike protein polypeptide is positioned N-terminal to the CD9 protein polypeptide. In some cases, a linker peptide is positioned between the spike protein polypeptide and the CD9 protein polypeptide, as depicted in FIG. 1A.

In some aspects in which the SARS-CoV-2 protein is a nucleocapsid protein and the exosomal tetraspanin protein is a CD9 protein, the nucleocapsid protein polypeptide is positioned N-terminal to the CD9 protein polypeptide. In some cases, a signal peptide is positioned at the N-terminal end of the nucleocapsid protein polypeptide. In some cases, a hinge peptide, a transmembrane domain peptide, and a linker peptide is positioned between the spike protein polypeptide and the CD9 protein polypeptide, as depicted in FIG. 2A.

In some aspects in which the SARS-CoV-2 protein is a spike protein, the SARS-CoV-2 spike protein polypeptide contains one or more mutations, such as, e.g., a furin cleavage site mutation (CSM [682RRAR685-to-682GSAG685]) and/or a di-proline substitution (2P [986KV987-to-986PP987]).

In one embodiment, a polynucleotide is provided that encodes a synthetic fusion protein that contains polypeptide sequences of a SARS-CoV-2 protein fused to polypeptide sequences of an exosomal tetraspanin protein. The encoded fusion protein is designed and made such that a SARS-CoV-2 protein antigen can be expressed in a cell and sorted onto the surface of an exosome to enable the eliciting of an immune response when that exosome is administered to a subject.

In one aspect, the encoded SARS-CoV-2 protein polypeptide is a spike protein polypeptide. In another aspect, the encoded SARS-CoV-2 protein polypeptide is a nucleocapsid protein polypeptide. In one aspect, the encoded exosomal tetraspanin protein polypeptide is a CD9 protein polypeptide.

In some aspects in which the encoded SARS-CoV-2 protein polypeptide is a spike protein polypeptide and the encoded exosomal tetraspanin protein polypeptide is a CD9 protein polypeptide, the encoded spike protein polypeptide is positioned N-terminal to the encoded CD9 protein polypeptide in the encoded synthetic fusion protein. In some cases, a linker peptide is encoded by the polynucleotide to be positioned between the spike protein polypeptide and the CD9 protein polypeptide of the encoded synthetic fusion protein, as depicted in FIG. 1A.

In some aspects in which the encoded SARS-CoV-2 protein polypeptide is a nucleocapsid protein polypeptide, and the encoded exosomal tetraspanin protein polypeptide is a CD9 protein polypeptide, the encoded nucleocapsid protein polypeptide is positioned N-terminal to the encoded CD9 protein polypeptide. In some cases, a signal peptide is positioned at the N-terminal end of the nucleocapsid protein polypeptide. In some cases, a hinge peptide, a transmembrane domain peptide, and a linker peptide are encoded by the polynucleotide to be positioned between the spike protein polypeptide and the CD9 protein polypeptide of the encoded synthetic fusion protein, as depicted in FIG. 2A.

In some aspects in which the encoded SARS-CoV-2 protein polypeptide is a spike protein polypeptide, the encoded SARS-CoV-2 spike protein polypeptide contains one or more mutations, such as, e.g., a furin cleavage site mutation (CSM [682RRAR685-to-682GSAG685]) and/or a di-proline substitution (2P [986KV987-to-986PP987]).

In one embodiment, a cell is provided that contains a polynucleotide that encodes a synthetic fusion protein that contains polypeptide sequences of a SARS-CoV-2 protein fused to polypeptide sequences of an exosomal tetraspanin protein. The encoded fusion protein is designed and made such that a SARS-CoV-2 protein antigen can be expressed in the cell and sorted onto the surface of an exosome to enable the eliciting of an immune response when that exosome is administered to a subject.

In some aspects, the cell is a metazoan cell. In some aspects, the cell is a vertebrate cell. In some aspects, the cell is a mammalian cell. In some aspects, the cell is a primate cell. In some aspects, the cell is a human cell. In some aspects, the cell is a primary cell. In some aspects, the cell is of an established cell line. In some aspects, the cell is a human embryonic kidney cell. In some aspects, the cell is a HEK293 cell. In some aspects, the cell is a 293F cell.

In one aspect, the polynucleotide in the cell encodes a SARS-CoV-2 protein polypeptide that is a spike protein polypeptide. In another aspect, the polynucleotide in the cell encodes a SARS-CoV-2 protein polypeptide that is a nucleocapsid protein polypeptide. In one aspect, the polynucleotide in the cell encodes an exosomal tetraspanin protein polypeptide that is a CD9 protein polypeptide.

In some aspects in which the polynucleotide in the cell encodes a SARS-CoV-2 protein polypeptide that is a spike protein polypeptide and encodes an exosomal tetraspanin protein polypeptide that is a CD9 protein polypeptide, the encoded spike protein polypeptide is positioned N-terminal to the encoded CD9 protein polypeptide in the encoded synthetic fusion protein. In some cases, the polynucleotide encodes a linker peptide sequence that is positioned between the spike protein polypeptide and the CD9 protein polypeptide of the encoded synthetic fusion protein, as depicted in FIG. 1A.

In some aspects in which the polynucleotide in the cell encodes a SARS-CoV-2 protein polypeptide that is a nucleocapsid protein polypeptide and encodes an exosomal tetraspanin protein polypeptide is a CD9 protein polypeptide, the encoded nucleocapsid protein polypeptide is positioned N-terminal to the encoded CD9 protein polypeptide. In some cases, the polynucleotide encodes a signal peptide that is positioned at the N-terminal end of the nucleocapsid protein polypeptide. In some cases, the polynucleotide encodes a hinge peptide, a transmembrane domain peptide, and a linker peptide that positioned between the spike protein polypeptide and the CD9 protein polypeptide of the encoded synthetic fusion protein, as depicted in FIG. 2A.

In some aspects in which the cell contains a polynucleotide that encodes a SARS-CoV-2 protein polypeptide that is a spike protein polypeptide, the encoded SARS-CoV-2 spike protein polypeptide contains one or more mutations, such as, e.g., a furin cleavage site mutation (CSM [682RRAR685-to-682GSAG685]) and/or a di-proline substitution (2P [986KV987-to-986PP987]).

In one embodiment, a cell is provided that contains a synthetic fusion protein that contains polypeptide sequences of a SARS-CoV-2 protein fused to polypeptide sequences of an exosomal tetraspanin protein. The synthetic fusion protein is designed and made such that a SARS-CoV-2 protein antigen can be expressed in the cell and sorted onto the surface of an exosome produced by the cell to enable the eliciting of an immune response when that exosome is administered to a subject.

In some aspects, the cell is a metazoan cell. In some aspects, the cell is a vertebrate cell. In some aspects, the cell is a mammalian cell. In some aspects, the cell is a primate cell. In some aspects, the cell is a human cell. In some aspects, the cell is a primary cell. In some aspects, the cell is of an established cell line. In some aspects, the cell is a human embryonic kidney cell. In some aspects, the cell is a HEK293 cell. In some aspects, the cell is a 293F cell.

In one aspect, the cell contains a SARS-CoV-2 protein polypeptide that is a spike protein polypeptide. In another aspect, the cell contains a SARS-CoV-2 protein polypeptide that is a nucleocapsid protein polypeptide. In one aspect, the cell contains an exosomal tetraspanin protein polypeptide that is a CD9 protein polypeptide.

In some aspects in which the cell contains a SARS-CoV-2 protein polypeptide that is a spike protein polypeptide and an exosomal tetraspanin protein is a CD9 protein, the spike protein polypeptide is positioned N-terminal to the CD9 protein polypeptide of the contained synthetic fusion protein. In some cases, a linker peptide is positioned between the spike protein polypeptide and the CD9 protein polypeptide of the contained synthetic fusion protein, as depicted in FIG. 1A.

In some aspects in which the cell contains a SARS-CoV-2 protein polypeptide that is a nucleocapsid protein polypeptide and an exosomal tetraspanin protein polypeptide that is a CD9 protein polypeptide, the nucleocapsid protein polypeptide is positioned N-terminal to the CD9 protein polypeptide. In some cases, a signal peptide is positioned at the N-terminal end of the nucleocapsid protein polypeptide of the contained synthetic fusion protein. In some cases, a hinge peptide, a transmembrane domain peptide, and a linker peptide is positioned between the spike protein polypeptide and the CD9 protein polypeptide of the contained synthetic fusion protein, as depicted in FIG. 2A.

In some aspects in which the cell contains a SARS-CoV-2 protein polypeptide that is a spike protein polypeptide, the SARS-CoV-2 spike protein polypeptide contains one or more mutations, such as, e.g., a furin cleavage site mutation (CSM [682RRAR685-to-682GSAG685]) and/or a di-proline substitution (2P [986KV987-to-986PP987]).

In one embodiment, a cell is provided that is produced by transducing the cell with a lentivirus containing a synthetic polynucleotide that encodes a synthetic fusion protein that contains polypeptide sequences of a SARS-CoV-2 protein fused to polypeptide sequences of an exosomal tetraspanin. The encoded fusion protein is designed and made such that a SARS-CoV-2 protein antigen can be expressed in a cell and sorted onto the surface of an exosome to enable the eliciting of an immune response when that exosome is administered to a subject.

In some aspects, the cell is a metazoan cell. In some aspects, the cell is a vertebrate cell. In some aspects, the cell is a mammalian cell. In some aspects, the cell is a primate cell. In some aspects, the cell is a human cell. In some aspects, the cell is a primary cell. In some aspects, the cell is of an established cell line. In some aspects, the cell is a human embryonic kidney cell. In some aspects, the cell is a HEK293 cell. In some aspects, the cell is a 293F cell.

In one aspect, the encoded SARS-CoV-2 protein polypeptide is a spike protein polypeptide. In another aspect, the encoded SARS-CoV-2 protein polypeptide is a nucleocapsid protein polypeptide. In one aspect, the encoded exosomal tetraspanin protein polypeptide is a CD9 protein polypeptide.

In some aspects in which the encoded SARS-CoV-2 protein polypeptide is a spike protein polypeptide and the encoded exosomal tetraspanin protein polypeptide is a CD9 protein polypeptide, the encoded spike protein polypeptide is positioned N-terminal to the encoded CD9 protein polypeptide in the encoded synthetic fusion protein. In some cases, a linker peptide is encoded by the polynucleotide to be positioned between the spike protein polypeptide and the CD9 protein polypeptide of the encoded synthetic fusion protein, as depicted in FIG. 1A.

In some aspects in which the encoded SARS-CoV-2 protein polypeptide is a nucleocapsid protein polypeptide, and the encoded exosomal tetraspanin protein polypeptide is a CD9 protein polypeptide, the encoded nucleocapsid protein polypeptide is positioned N-terminal to the encoded CD9 protein polypeptide. In some cases, a signal peptide is positioned at the N-terminal end of the nucleocapsid protein polypeptide. In some cases, a hinge peptide, a transmembrane domain peptide, and a linker peptide are encoded by the polynucleotide to be positioned between the spike protein polypeptide and the CD9 protein polypeptide of the encoded synthetic fusion protein, as depicted in FIG. 2A.

In some aspects in which the encoded SARS-CoV-2 protein polypeptide is a spike protein polypeptide, the encoded SARS-CoV-2 spike protein polypeptide contains one or more mutations, such as, e.g., a furin cleavage site mutation (CSM [682RRAR685-to-682GSAG685]) and/or a di-proline substitution (2P [986KV987-to-986PP987]).

In one embodiment, a vesicle is provided that contains a synthetic fusion protein that contains polypeptide sequences of a SARS-CoV-2 protein fused to polypeptide sequences of an exosomal tetraspanin protein. The fusion protein is designed and made such that a SARS-CoV-2 protein antigen can be expressed on the surface of an exosome to enable the eliciting of an immune response when administered to a subject.

In one aspect, the vesicle contains a SARS-CoV-2 protein polypeptide that is a spike protein polypeptide. In another aspect, the vesicle contains a SARS-CoV-2 protein polypeptide that is a nucleocapsid protein polypeptide. In one aspect, the vesicle contains an exosomal tetraspanin protein polypeptide that is a CD9 protein.

In some aspects in which the vesicle contains a SARS-CoV-2 protein polypeptide that is a spike protein and an exosomal tetraspanin protein polypeptide that is a CD9 protein, the spike protein polypeptide is positioned N-terminal to the CD9 protein polypeptide. In some cases, a linker peptide is positioned between the spike protein polypeptide and the CD9 protein polypeptide, as depicted in FIG. 1A.

In some aspects in which the vesicle contains a SARS-CoV-2 protein polypeptide that is a nucleocapsid protein and an exosomal tetraspanin protein polypeptide that is a CD9 protein, the nucleocapsid protein polypeptide is positioned N-terminal to the CD9 protein polypeptide. In some cases, a signal peptide is positioned at the N-terminal end of the nucleocapsid protein polypeptide. In some cases, a hinge peptide, a transmembrane domain peptide, and a linker peptide is positioned between the spike protein polypeptide and the CD9 protein polypeptide, as depicted in FIG. 2A.

In some aspects in which the vesicle contains a SARS-CoV-2 protein polypeptide that is a spike protein, the SARS-CoV-2 spike protein polypeptide contains one or more mutations, such as, e.g., a furin cleavage site mutation (CSM [682RRAR685-to-682GSAG685]) and/or a di-proline substitution (2P [986KV987-to-986PP987]).

In one aspect, the vesicle has a diameter of about 50-500 nm. In one aspect, the vesicle is a synthetic vesicle. In one aspect, the vesicle is produced by a cell. In one aspect, the vesicle is an extracellular vesicle. In one aspect, the vesicle is a microvesicle. In one aspect, the vesicle is an exosome. In one aspect, the vesicle is an apoptotic body. In one aspect, the vesicle expresses a CD81 protein on its surface.

In some aspects, the lumen of the vesicle contains a cargo molecule. Here, in some cases, the protein or polypeptide that is displayed on the surface is a targeting ligand that serves to target the vesicle to a receiving target to which the cargo molecule is delivered. In some aspects, the cargo molecule is a nucleic acid, such as, e.g., a PMO antisense oligonucleotide, a peptide, polypeptide, or protein, a hydrophobic small molecule drug, a hydrophilic small molecule drug, an imaging agent, an aptamer, a trap molecule, a nanobody, an antibody of fragment thereof, a receptor tyrosine kinase, and/or the like. For example, a spike-expressing vesicle may contain an immune effector molecule cargo.

In one embodiment, a method is provided for making a vesicle that contains a synthetic fusion protein that contains polypeptide sequences of a SARS-CoV-2 protein fused to polypeptide sequences of an exosomal tetraspanin protein. The fusion protein is designed and made such that a SARS-CoV-2 protein antigen can be expressed on the surface of an exosome to enable the eliciting of an immune response when administered to a subject. Here, a cell containing a polynucleotide that encodes the synthetic fusion protein is cultured in a cell culture medium such that vesicles are secreted by the cell into the medium, the cell culture medium is then collected, and the vesicles subsequently purified.

In some aspects, the cell that is cultured to produce the vesicle is a metazoan cell. In some aspects, the cell is a vertebrate cell. In some aspects, the cell is a mammalian cell. In some aspects, the cell is a primate cell. In some aspects, the cell is a human cell. In some aspects, the cell is a primary cell. In some aspects, the cell is of an established cell line. In some aspects, the cell is a human embryonic kidney cell. In some aspects, the cell is a HEK293 cell. In some aspects, the cell is a 293F cell.

In some aspects, expression of the polynucleotide to produce the synthetic fusion protein is constitutive. In other aspects, expression of the polynucleotide is induced in the cell to produce the synthetic fusion protein. Here, in one aspect, induction is effected by contacting the cell with tetracycline, doxycycline, or analogs thereof. Here, in another aspect, induction is effected by removing from the cell or cell medium tetracycline, doxycycline, or analogs thereof.

In one aspect, the polynucleotide encodes and expresses in the cell a SARS-CoV-2 protein polypeptide that is a spike protein polypeptide. In another aspect, the polynucleotide encodes and expresses in the cell a SARS-CoV-2 protein polypeptide that is a nucleocapsid protein polypeptide. In one aspect, the polynucleotide encodes and expresses in the cell encodes an exosomal tetraspanin protein polypeptide that is a CD9 protein polypeptide.

In some aspects in which the polynucleotide in the cell encodes a SARS-CoV-2 protein polypeptide that is a spike protein polypeptide and encodes an exosomal tetraspanin protein polypeptide that is a CD9 protein polypeptide, the spike protein polypeptide is positioned N-terminal to the CD9 protein polypeptide in the encoded and expressed synthetic fusion protein. In some cases, the polynucleotide encodes a linker peptide sequence that is positioned between the spike protein polypeptide and the CD9 protein polypeptide of the encoded and expressed synthetic fusion protein, as depicted in FIG. 1A.

In some aspects in which the polynucleotide in the cell encodes a SARS-CoV-2 protein polypeptide that is a nucleocapsid protein polypeptide and encodes an exosomal tetraspanin protein polypeptide is a CD9 protein polypeptide, the encoded nucleocapsid protein polypeptide is positioned N-terminal to the encoded CD9 protein polypeptide in the encoded and expressed fusion protein. In some cases, the polynucleotide encodes a signal peptide that is positioned at the N-terminal end of the nucleocapsid protein polypeptide. In some cases, the polynucleotide encodes a hinge peptide, a transmembrane domain peptide, and a linker peptide that positioned between the spike protein polypeptide and the CD9 protein polypeptide of the encoded and expressed synthetic fusion protein, as depicted in FIG. 2A.

In some aspects in which the cell contains a polynucleotide that encodes and expresses a SARS-CoV-2 protein polypeptide that is a spike protein polypeptide as part of the expressed fusion protein, the encoded and expressed SARS-CoV-2 spike protein polypeptide contains one or more mutations, such as, e.g., a furin cleavage site mutation (CSM [682RRAR685-to-682GSAG685]) and/or a di-proline substitution (2P [986KV987-to-986PP987]).

In one embodiment, an immunogenic composition containing a plurality of vesicles that contain synthetic fusion proteins is provided. Here, the synthetic fusion protein contains a polypeptide sequence of a SARS-CoV-2 protein fused to a polypeptide sequence of an exosomal tetraspanin protein. The fusion protein is designed and made such that a SARS-CoV-2 protein antigen can be expressed on the surface of an exosome to enable the eliciting of an immune response when administered to a subject.

In some aspects, the immunogenic composition comprises (i) a first plurality of containing a first synthetic fusion protein that contains a polypeptide sequence of a first SARS-CoV-2 protein fused to a polypeptide sequence of an exosomal tetraspanin protein and (ii) a second plurality of vesicles containing a second synthetic fusion protein that contains a polypeptide sequence of a second SARS-CoV-2 protein fused to a polypeptide sequence of an exosomal tetraspanin protein. For example, in one aspect, the polypeptide sequence of the first SARS-CoV-2 protein is a spike protein polypeptide sequence, and the polypeptide sequence of the second SARS-CoV-2 protein is a nucleocapsid protein polypeptide sequence.

In one aspect, immunogenic composition contains a plurality of vesicles that contain synthetic fusion proteins in which the SARS-CoV-2 protein polypeptide is a spike protein polypeptide. In another aspect, immunogenic composition contains a plurality of vesicles that contain synthetic fusion proteins in which the SARS-CoV-2 protein polypeptide is a nucleocapsid protein polypeptide. In another aspect, immunogenic composition contains a plurality of vesicles that contain a combination of vesicles, some of which contain a synthetic fusion protein in which the SARS-CoV-2 protein polypeptide is a spike protein polypeptide, and others of which contain a synthetic fusion protein in which the SARS-CoV-2 protein polypeptide is a nucleocapsid protein polypeptide. In one aspect, immunogenic composition contains a plurality of vesicles that contain synthetic fusion proteins in which the exosomal tetraspanin protein polypeptide is a CD9 protein polypeptide.

In some aspects in which the immunogenic composition contains a plurality of vesicles that contain synthetic fusion proteins in which the SARS-CoV-2 protein polypeptide is a spike protein polypeptide and the exosomal tetraspanin protein polypeptide is a CD9 protein polypeptide, the spike protein polypeptide is positioned N-terminal to the CD9 protein polypeptide. In some cases, a linker peptide is positioned between the spike protein polypeptide and the CD9 protein polypeptide, as depicted in FIG. 1A.

In some aspects in which the immunogenic composition contains a plurality of vesicles that contain synthetic fusion proteins in which the SARS-CoV-2 protein polypeptide SARS-CoV-2 protein is a nucleocapsid protein polypeptide and the exosomal tetraspanin protein polypeptide is a CD9 protein, the nucleocapsid protein polypeptide is positioned N-terminal to the CD9 protein polypeptide. In some cases, a signal peptide is positioned at the N-terminal end of the nucleocapsid protein polypeptide. In some cases, a hinge peptide, a transmembrane domain peptide, and a linker peptide is positioned between the spike protein polypeptide and the CD9 protein polypeptide, as depicted in FIG. 2A.

In some aspects in which immunogenic composition contains a plurality of vesicles that contain synthetic fusion proteins in which the SARS-CoV-2 protein polypeptide is a spike protein polypeptide, the SARS-CoV-2 spike protein polypeptide contains one or more mutations, such as, e.g., a furin cleavage site mutation (CSM [682RRAR685-to-682GSAG685]) and/or a di-proline substitution (2P [986KV987-to-986PP987]).

In some aspects, the immunogenic composition contains a plurality of vesicles containing the synthetic fusion protein at a concentration of about 2E9 vesicles/mL-3E13 vesicles/mL. In those aspects in which the immunogenic composition contains two types of vesicles, i.e., one type containing a first synthetic fusion protein and another type containing a second synthetic fusion protein, each vesicle type may be provided at a concentration of about 1E9 vesicles/mL-2E13 vesicles/mL or 2E9 vesicles/mL-3E13 vesicles/mL.

In some aspects, the immunogenic composition contains a plurality of vesicles containing the synthetic fusion protein at a concentration of about 0.3 ng/mL-3 μg/mL of the synthetic fusion protein. In those aspects in which the immunogenic composition contains two types of vesicles, i.e., one type containing a first synthetic fusion protein and another type containing a second synthetic fusion protein, each vesicle type may be provided at a fusion protein concentration of about 0.1 ng/mL-1.5 μg/mL or about 0.3 ng/mL-3 μg/mL.

In some aspects, the immunogenic composition further contains one or more pharmaceutically acceptable excipients. In one aspect, the immunogenic composition does not contain an adjuvant.

In some aspects, a vesicle of the plurality of vesicles of the immunogenic composition has an average diameter of about 50-500 nm. In one aspect, the vesicle is a synthetic vesicle. In one aspect, the vesicle is produced by a cell. In one aspect, the vesicle is an extracellular vesicle. In one aspect, the vesicle is a microvesicle. In one aspect, the vesicle is an exosome. In one aspect, the vesicle is an apoptotic body. In one aspect, the vesicle expresses a CD81 protein on its surface.

In one embodiment, a method of eliciting an immune response in a subject is provided by administering to the subject a dose of an immunogenic composition described in the preceding embodiment. In some aspects, the subject is administered more than one dose, such as a second dose administered a period of time after the first dose, and/or subsequent booster doses. In some embodiments, the period of time (e.g., between doses) is 14 days-1 year.

In one aspect, a dose contains about 100 μL-1 mL of the immunogenic composition. In some aspects, the immunogenic composition contains about 0.1 ng/mL-3 μg/mL of synthetic fusion protein in a form expressed on the surface of vesicles. In some aspects, the immunogenic composition contains about 2.81E9 vesicles/mL-2.81E13 vesicles/mL that express synthetic fusion protein on their surfaces.

In some aspects, the immune response that is elicited in the subject is the production of neutralizing antibodies against a virus, such as SARS-CoV-2, influenza, and the like. In some aspects, the immune response that is elicited in the subject is the production of neutralizing antibodies against two or more SARS-CoV-2 variants, such as for example a delta variant, an omicron variant, or other variant of interest or concern now known or yet to be discovered. In some aspects, the immune response that is elicited in the subject is the production of anti-spike antibodies. In some aspects, the immune response that is elicited in the subject is the production of anti-nucleocapsid antibodies. In some aspects, the immune response that is elicited in the subject is a spike-specific T cell response, such as CD4+ and/or CD8+ response. In some aspects, the immune response that is elicited in the subject is a nucleocapsid-specific T cell response, such as CD4+ and/or CD8+ response.

### DRAWINGS

FIG. 1A depicts a linear cartoon of a SARS-CoV-2 spike CD9 fusion protein oriented from left-to-right amino-terminus-to-carboxy-terminus with a linker sequence positioned between the spike protein polypeptide and the CD9 protein polypeptide.

FIG. 1B depicts a cartoon of a SARS-CoV-2 spike CD9 fusion protein of FIG. 1A relative to a vesicle membrane oriented from left-to-right amino-terminus-to-carboxy-terminus. The spike protein polypeptide spans the membrane once with its amino terminus oriented on the outside of the vesicle. The linker is positioned on the luminal side of the membrane. The CD9 protein polypeptide spans the membrane 4 times with its carboxy terminus oriented in the lumen of the vesicle.

FIG. 2A depicts a linear cartoon of a SARS-CoV-2 nucleocapsid CD9 fusion protein oriented from left-to-right amino-terminus-to-carboxy-terminus with an amino terminal signal peptide fused to a nucleocapsid protein polypeptide fused to a hinge region peptide fused to a transmembrane domain peptide fused to a linker peptide fused to a CD9 protein polypeptide.

FIG. 2B depicts a cartoon of a SARS-CoV-2 nucleocapsid CD9 fusion protein of FIG. 2A relative to a vesicle membrane oriented from left-to-right amino-terminus-to-carboxy-terminus. The nucleocapsid protein polypeptide with the amino terminal signal sequence is positioned on the outside (cytoplasmic or external side) with a linker sequence connecting the nucleocapsid protein polypeptide to the transmembrane domain peptide, which spans the membrane and which in turn connects to the linker positioned in the lumen and which connects to the CD9 protein polypeptide which spans the membrane 4 times with its carboxy terminus oriented in the lumen of the vesicle.

FIG. 3A is a flow chart depicting the elements and steps for producing cells expressing a spike protein polypeptide fusion protein using a packaging cell (301) and a host cell (311) mediated by a lentivirus vector (304).

FIG. 3B is a histogram depicting relative fluorescent intensity flow analysis of host cells expressing spike protein on their surface.

FIG. 4A is a graph depicting the concentration of spike-expressing exosomes per milliliter as a function of exosome diameter in nanometers.

FIG. 4B is a western blot stained for SARS-CoV-2 spike protein. The first column from left to right depicts lane loaded with size markers, second column represents lane loaded with non-transduced 293F (host cell) protein, third column represents lane loaded with protein from exosomes derived from non-transduced 293F cells, fourth column represents lane loaded with protein from 293F cells constitutively expressing spike fusion protein, fifth column represents lane loaded with protein from exosomes derived from 293F cells constitutively expressing spike fusion protein, and sixth column represents lane loaded with spike fusion protein.

FIG. 4C is a histogram depicting relative fluorescent intensity flow analysis of exosomes with spike expressed on the surface. The left curve represents exosomes derived from 293F cells that do not express a spike-CD9 fusion protein. The right curve represents exosomes derived from 293F cells expressing a spike-CD9 fusion protein.

FIG. 5A depicts a transmission electron micrograph of exosomes expressing SARS-CoV-2 spike protein. Inset is a blow up showing an exosome decorated with spike protein. Arrows point to SARS-CoV-2 spikes around the circumference of exosomes.

FIG. 5B depicts a higher magnification transmission electron micrograph of exosomes expressing SARS-CoV-2 spike protein. Arrows point to SARS-CoV-2 spikes around the circumference of exosomes.

FIG. 6A is a graph depicting the concentration of nucleocapsid-expressing exosomes per milliliter as a function of exosome diameter in nanometers.

FIG. 6B is a western blot stained for SARS-CoV-2 nucleocapsid protein. The first column from left to right depicts lane loaded with size markers, second column represents lane loaded with nucleocapsid protein, third column represents lane loaded with protein from exosomes derived from non-transduced 293F cells, fourth column represents lane loaded with protein from non-transduced 293F cells, and fifth column represents lane loaded with protein from exosomes derived from 293F cells expressing nucleocapsid fusion protein.

FIG. 6C is a histogram depicting relative fluorescent intensity flow analysis of exosomes with or without nucleocapsid protein expressed on the surface. Moving from left to right, the first curve represents exosomes derived from unmodified 293F cells, the second curve represents exosomes derived from uninduced 293F cells harboring a nucleocapsid-CD9 fusion protein-encoding polynucleotide, the third curve represents exosomes derived from 293F cells expressing nucleocapsid-CD9 fusion protein, the smaller third curve represents exosomes derived from 293F cells expressing nucleocapsid-CD9 fusion protein under tet induction, the larger third curve represents exosomes derived from 293F cells harboring a CD9 knock-out and expressing nucleocapsid-CD9 fusion protein.

FIG. 7 is a timeline depicting a process for generating am immune response from administering an immunogenic composition containing exosomes expressing SARS-CoV-2 fusion proteins on their surface.

FIGS. 8A-8D. STX-S exosome elicited a robust immune response using significantly lower antigen than recombinant protein vaccines. Data are shown as mean±SEM. **** p<0.0005, ***p<0.001, **p<0.01, *p<0.05, ns=not significant, *1-way ANOVA; ##p<0.01, #p<0.05, #—2 tail-t test.

FIG. 8A is a histogram depicting fold change in murine day-14 1:100-diluted serum antibody titer against spike protein as a function of exosome-expressing spike dosage in nanograms. The left-most bar represents PBS control; the second bar represents a 10 ng dose of spike expressing exosomes (STX-S); the third bar represents a 32 ng dose of STX-S; the right most bar represents sera from mice injected with 32 ng spike protein dose combined with adjuvant.

FIG. 8B is a histogram depicting fold change in murine day-35 1:100-diluted serum antibody titer against spike protein as a function of exosome-expressing spike dosage in nanograms. The left-most bar represents PBS control; the second bar represents a 10 ng dose of spike expressing exosomes (STX-S); the third bar represents a 32 ng dose of STX-S; the right most bar represents sera from mice injected with 32 ng spike protein dose combined with adjuvant.

FIG. 8C is a bar graph depicting an ELISpot assay showing the number of IL4 producing spots responding to spike protein as a function of antigen dose. Moving from left to right on the x-axis, the first bar represents non-spike-containing wells from PBS control splenocytes; the second bar represents spike protein reactivity from PBS control splenocytes; the third bar represents non-spike-containing wells from splenocytes of 10 ng STX-S dosed subject; the fourth bar represents spike protein reactivity from splenocytes of 10 ng STX-S dosed subject; the fifth bar represents non-spike-containing wells from splenocytes of 32 ng STX-S dosed subject; the sixth bar represents spike protein reactivity from splenocytes of 32 ng STX-S dosed subject; the seventh bar represents non-spike-containing wells from splenocytes of 32 ng spike protein with adjuvant dosed subject; the eighth bar represents spike protein reactivity from splenocytes of 32 ng spike protein with adjuvant dosed subject.

FIG. 8D is a bar graph depicting an ELISpot assay showing the number of IFNγ producing spots responding to spike protein as a function of antigen dose. Moving from left to right on the x-axis, the first bar represents non-spike-containing wells from PBS control splenocytes; the second bar represents spike protein reactivity from PBS control splenocytes; the third bar represents non-spike-containing wells from splenocytes of 10 ng STX-S dosed subject; the fourth bar represents spike protein reactivity from splenocytes of 10 ng STX-S dosed subject; the fifth bar represents non-spike-containing wells from splenocytes of 32 ng STX-S dosed subject; the sixth bar represents spike protein reactivity from splenocytes of 32 ng STX-S dosed subject; the seventh bar represents non-spike-containing wells from splenocytes of 32 ng spike protein with adjuvant dosed subject; the eighth bar represents spike protein reactivity from splenocytes of 32 ng spike protein with adjuvant dosed subject.

FIG. 9, panels A-D. STX-N vaccine elicits a multivalent immune-response. Data are shown as mean±SEM. *p<0.05, ***p<0.005, ****p<0.001, ANOVA, corrected multiple comparisons; ns=not significant. Dose 1=0.32 ng N per injection; dose 2=3.2 ng N per injection; dose 3=10 ng N per injection

Panels 9A and 9B: Histograms depicting anti-nucleocapsid antibody titers of day-35 post immunization sera as a function of dose. STX-N vaccine induced modest expression of SARS-CoV-2 Nucleocapsid antibodies in two sample bins (N 1 and N 2). PBS was used as a vehicle control.

Panels 9C and 9D: Histograms depicting anti-nucleocapsid IFNγ ELISpot positive wells of day-40 post immunization splenocytes as a function of dose.

FIGS. 10, panels A-C: Line graphs depicting percent SARS-CoV-2 neutralization as a function of immune sera at escalating serum dilution. The graphs depict the production of neutralizing antibodies after STX-S injection. Data are shown as mean±SEM. COV-02-Delta=plasma from a patient immunized with Moderna's mRNA vaccine with a breakthrough SARS-CoV-2 delta spike infection. Dose 4=9.8 ng S per injection; dose 2=3.2 ng S per injection.

Panel 10A. STX-S vaccine generated strong neutralization against SARS-CoV-2 delta spike (B.1.617.2)

Panel 10B. STX-S vaccine resulted in neutralization of SARS-CoV-2 spike Omicron BA.1

Panel 10C. STX-S vaccine resulted in neutralization of SARS-CoV-2 spike Omicron BA.5.2.1.

FIG. 11 is a histogram depicting fold change in anti-spike antibody titer from mice as a function of time and dosage. Data are shown as mean±SEM. *p<0.05, ***p<0.005, ****p<0.001, ANOVA, corrected multiple comparisons; ns=not significant. From left to right, the first histogram represents sera from mice dosed with PBS, the second histogram represents day 14 sera from mice dosed with a single dose of 10 ng STX-S (exosomes expressing CD9-spike fusion), the third histogram represents day 35 sera from mice dosed with a first dose of 10 ng STX-S followed by a 7 ng booster at day 21, the fourth histogram represents day 61 sera from mice dosed with a first dose of 10 ng STX-S followed by a 7 ng booster at day 21, the fifth histogram represents day 89 sera from mice dosed with a first dose of 10 ng STX-S followed by a 7 ng booster at day 21, the sixth histogram represents day 123 sera from mice dosed with a first dose of 10 ng STX-S followed by a 7 ng booster at day 21, the seventh histogram represents day 151 sera from mice dosed with a first dose of 10 ng STX-S followed by a 7 ng booster at day 21, the eighth histogram represents day 278 sera from mice dosed with a first dose of 10 ng STX-S followed by a 7 ng booster at day 21, the ninth histogram represents day 166 sera from mice dosed with a first dose of 10 ng STX-S followed by a 7 ng booster at day 21, the tenth histogram represents day 166 plus month 4 follow-up sera from mice dosed with a first dose of 10 ng STX-S followed by a 7 ng booster at day 21, the eleventh histogram represents day 166 sera from mice dosed with a first dose of 10 ng STX-S followed by a 3 ng booster at day 21, and the twelfth histogram represents day 166 plus month 4 follow-up sera from mice dosed with a first dose of 10 ng STX-S followed by a 3 ng booster at day 21.

FIG. 12 is a line graph depicting the percent uptake of exosomes by HEK293-hACE2 cells as a function of exosome concentration expressed in number of exosomes per mL. Series 1 (blue) represents HEK293-hACE2 cell-uptake of 293F exosomes that do not express spike protein. Series 2 (orange) represents HEK293-hACE2 cell-uptake of spike-expressing exosomes.

FIG. 13 are histograms depicting relative fluorescent intensity flow analysis of exosomes with spike expressed on the surface (Panel 13A) or exosomes with nucleocapsid expressed on the surface (Panel 13B). For each panel, the left curve represents exosomes derived from 293F cells that do not express a spike (A) or nucleocapsid (B). For each panel, the right curve represents exosomes derived from 293F cells expressing a spike (A) or nucleocapsid (B).

FIG. 14 is a line graph depicting size distribution of STX-S and STX-N exosomes as a function of exosome concentration by ZetaView nanoparticle tracking analysis (NTA). Blue line represents spike expressing exosomes. Green line represents nucleocapsid expressing exosomes. Gray line represents non-engineered 293F derived exosomes.

FIG. 15 depicts TEM images of purified STX-S (panel A; bar=200 nm) and STX-N (panel B; bar=500 nm) exosomes.

FIG. 16 is a JESS western blot stained for SARS-CoV-2 spike protein. The first column from left to right depicts lane loaded with size markers, second column represents lane loaded with protein from non-transduced 293F cells, third column represents lane loaded with protein from exosomes derived from non-transduced 293F cells, fourth column represents 293F cells expressing spike fusion protein, and fifth column represents lane loaded with protein from exosomes derived from 293F cells expressing nucleocapsid fusion protein. 0.8 ug protein was loaded per lane, as calculated by BCA assay.

FIG. 17 is a JESS western blot stained for SARS-CoV-2 nucleocapsid protein. The first column from left to right depicts lane loaded with size markers, second column represents lane loaded with protein from non-transduced 293F cells, third column represents lane loaded with protein from exosomes derived from non-transduced 293F cells, fourth column represents 293F cells expressing spike fusion protein, and fifth column represents lane loaded with protein from exosomes derived from 293F cells expressing nucleocapsid fusion protein. 0.8 ug protein was loaded per lane, as calculated by BCA assay.

FIG. 18A is a histogram depicting fold change in murine day-14 serum antibody titer against spike protein after 1 i.m. injection as a function of spike dosage formulation. The left-most bar represents PBS control; the second bar represents a dose of spike expressing exosomes (STX S); the third bar represents an equivalent dose spike (S) combined with adjuvant. N=10/experimental group. Data are shown as mean±SEM. **** p<0.0005, ***p<0.001, **p<0.01, ns=not significant, 1-way ANOVA.

FIG. 18B is a histogram depicting fold change in murine day-35 1:100-diluted serum antibody titer against spike protein after 2 i.m. injections as a function of spike dosage formulation. The left-most bar represents PBS control; the second bar represents a dose of spike expressing exosomes (STX S); the third bar represents an equivalent dose spike (S) combined with adjuvant. N=10/experimental group. Data are shown as mean±SEM. **** p<0.0005, ***p<0.001, **p<0.01, ns=not significant, 1-way ANOVA.

FIG. 19A is a histogram depicting fold change in murine day-14 serum antibody titer against nucleocapsid protein after 1 i.m. injection as a function of nucleocapsid dosage formulation. The left-most bar represents PBS control; the second bar represents a dose of nucleocapsid expressing exosomes (STX S); the third bar represents an equivalent dose nucleocapsid (N) combined with adjuvant. N=10/experimental group. Data are shown as mean SEM. **** p<0.0005, ***p<0.001, **p<0.01, ns=not significant, 1-way ANOVA.

FIG. 19B is a histogram depicting fold change in murine day-35 serum antibody titer against spike protein after 2 i.m. injections as a function of spike dosage formulation. The left-most bar represents PBS control; the second bar represents a dose of spike expressing exosomes (STX S); the third bar represents an equivalent dose spike (S) combined with adjuvant. N=10/experimental group. Data are shown as mean±SEM. **** p<0.0005, ***p<0.001, **p<0.01, ns=not significant, 1-way ANOVA.

FIG. 20 is a histogram showing STX-S+N combination vaccine eliciting a strong antibody mice. Panel A represents IgG against Spike at day 14. Panel B represents IgG against Spike at day 35. For both panels A and B, the first column depicts IgG fold change due to PBS. Column 2 depicts IgG fold change due to S+N at dose 1 (25 ng S, 2.5 ng N). Column 3 depicts IgG fold change due to S+N at dose 2 (10 ng S, 4 ng N). Column 4 depicts IgG fold change due to S+N at dose 3 (3 ng S, 9 ng N). Data are shown as mean±SEM. *p<0.05, **p<0.01, ***p<0.005, **** p<0.001, ns=not significant, 1-way ANOVA, adjusted for multiple comparison. N=10 animals per experimental group.

FIG. 21 is a histogram showing STX-S+N combination vaccine eliciting a strong antibody mice. Panel A represents IgG against Nucleocapsid at day 14. Panel B represents IgG against Nucleocapsid at day 35. For both panels A and B, the first column depicts IgG fold change due to PBS. Column 2 depicts IgG fold change due to S+N at dose 1 (25 ng S, 2.5 ng N). Column 3 depicts IgG fold change due to S+N at dose 2 (10 ng S, 4 ng N). Column 4 depicts IgG fold change due to S+N at dose 3 (3 ng S, 9 ng N). Data are shown as mean±SEM. *p<0.05, **p<0.01, ***p<0.005, **** p<0.001, ns=not significant, 1-way ANOVA, adjusted for multiple comparison. N=10 animals per experimental group.

FIG. 22 are histograms depicting anti-spike IFNγ ELISpot positive wells (panel A) and anti-nucleocapsid IFNγ ELISpot positive wells (panel B) of day-40 post immunization splenocytes as a function of dose of spike-expressing exosomes (STX-S) plus nucleocapsid-expressing exosomes (STX-N). Here, S=spike protein; N=nucleocapsid protein; dose 1=25 ng S, 2.5 ng N; dose 2=10 ng S, 4 ng N; dose 3=3 ng S, 9 ng N. Data are shown as mean SEM. *p<0.05, **p<0.01, ***p<0.005, **** p<0.001, ns=not significant, 1-way ANOVA, adjusted for multiple comparison. N=10 animals per experimental group.

FIG. 23 are histograms depicting change in anti-spike antibody titer (Panel A) or change in anti-nucleocapsid antibody titer (Panel B) as a function of dose of combination vaccine STX-S+N, wherein dose 1 is 125 ng spike+10 ng nucleocapsid, and dose 2 is 50 ng spike+20 ng nucleocapsid. For each antibody read-out and dose form, the first bar, from left to right, represents serum from blood collected at day-7 post first dose; the second bar, day-14 post first dose; the third bar day-7 post second dose, day 21 post first dose; and the fourth bar day-14 post second dose, day 28 post first dose. Data are shown as mean±SEM. N=8 animals per experimental group.

FIG. 24, panels A-C: Line graphs depicting percent SARS-CoV-2 neutralization as a function of immune sera at escalating serum dilution. The graphs depict the production of neutralizing antibodies after STX-S+N (combination vaccine) injection. Data are shown as mean SEM. Data are shown as mean±SEM. COV-02-Delta=plasma from a patient immunized with Moderna's mRNA vaccine with a breakthrough SARS-CoV-2 delta spike infection; CoV-01=plasma from a patient who received two doses of the Moderna covid-19 vaccine with no prior history of SARS-CoV-2 infection. N=8 animals per experimental group.

Panel 24A. STX-S vaccine generated strong neutralization against SARS-CoV-2 delta spike (B.1.617.2)

Panel 24B. STX-S vaccine resulted in neutralization of SARS-CoV-2 spike Omicron BA.1

Panel 24C. STX-S vaccine resulted in neutralization of SARS-CoV-2 spike Omicron BA.5.2.1.

FIG. 25 are histograms depicting anti-spike IFNγ ELISpot positive wells (panel A) and anti-nucleocapsid IFNγ ELISpot positive wells (panel B) of day-28 post immunization rabbit splenocytes as a function of dose of combination vaccine including spike-expressing exosomes (STX-S) plus nucleocapsid-expressing exosomes (STX-N). Here, S=spike protein; N=nucleocapsid protein; dose 1=125 ng S, 10 ng N; dose 2=50 ng S, 20 ng N. Data are shown as mean±SEM. N=8 animals per experimental group.

### DETAILED DESCRIPTION OF EMBODIMENTS

### Definitions

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

As used herein, “and/or” refers to and encompasses any and all possible combinations of one or more of the associated listed items, as well as the lack of combinations when interpreted in the alternative (or).

The terms “about” and “approximate”, as used herein when referring to a measurable value such as an amount, dose, time, temperature, activity, level, number, frequency, percentage, dimension, size, amount, weight, position, length and the like, is meant to encompass variations of ±15%, ±10%, 5%, ±1%, ±0.5%, or even ±0.1% of the specified amount, dose, time, temperature, activity, level, number, frequency, percentage, dimension, size, amount, weight, position, length and the like. In instances in which the terms “about” and “approximate” are used in connection with the location or position of regions within a reference polypeptide, these terms encompass variations of ±up to 20 amino acid residues, ±up to 15 amino acid residues, ±up to 10 amino acid residues, ±up to 5 amino acid residues, ±up to 4 amino acid residues, ±up to 3 amino acid residues, ±up to 2 amino acid residues, or even ±1 amino acid residue.

The term “derived from” as in “A is derived from B” means that A is obtained from B in such a manner that A is not identical to B.

The terms “treat”, “therapeutic”, “prophylactic” and “prevent” are not intended to be absolute terms. Treatment, prevention and prophylaxis can refer to any delay in onset, amelioration of symptoms, improvement in patient survival, increase in survival time or rate, etc. Treatment, prevention, and prophylaxis can be complete or partial. The term “prophylactic” means not only “prevent”, but also minimize illness and disease. For example, a “prophylactic” agent can be administered to a subject, e.g., a human subject, to prevent infection, or to minimize the extent of illness and disease caused by such infection. The effect of treatment can be compared to an individual or pool of individuals not receiving the treatment, or to the same patient prior to treatment or at a different time during treatment. In some aspects, the severity of disease is reduced by at least 10%, as compared, e.g., to the individual before administration or to a control individual not undergoing treatment. In some aspects, the severity of disease is reduced by at least 25%, 50%, 75%, 80%, or 90%, or in some cases, no longer detectable using standard diagnostic techniques.

A treatment can be considered “effective,” as used herein, if one or more of the signs or symptoms of a condition described herein are altered in a beneficial manner, other clinically accepted symptoms are improved, or even ameliorated, or a desired response is induced e.g., by at least 2%, 3%, 4%, 5%, 10%, or more, following treatment according to the methods described herein. Efficacy can be assessed, for example, by measuring a marker, indicator, symptom, and/or the incidence of a condition treated according to the methods described herein or any other measurable parameter appropriate. Efficacy can also be measured by a failure of an individual to worsen as assessed by hospitalization, or need for medical interventions (e.g., progression of the disease is halted). Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. One skilled in the art can monitor efficacy of administration and/or treatment by measuring any one of such parameters, or any combination of parameters.

The term “effective amount” as used herein refers to the amount of a composition or an agent needed to alleviate at least one or more symptom of the disease or disorder, and relates to a sufficient amount of therapeutic composition to provide the desired effect. The term “therapeutically effective amount” refers to an amount of a composition or therapeutic agent that is sufficient to provide a particular effect when administered to a typical subject. An effective amount as used herein, in various contexts, can include an amount sufficient to delay the development of a symptom of the disease, alter the course of a symptom disease (for example but not limited to, slowing the progression of a symptom of the disease), or reverse a symptom of the disease. For example, for the given parameter, a therapeutically effective amount will show an increase or decrease of therapeutic effect at least any of 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least any of a 1.2-fold, 1.5-fold, 2-fold, 5-fold, or more effect over a control. The therapeutically effective amount may be administered in one or more doses of the therapeutic agent. The therapeutically effective amount may be administered in a single administration, or over a period of time in a plurality of doses.

“Administering” as used herein can include any suitable routes of administering a therapeutic agent or composition as disclosed herein. Suitable routes of administration include, without limitation, oral, parenteral, intravenous, intramuscular, subcutaneous, transdermal, airway (aerosol), pulmonary, cutaneous, injection or topical administration. Administration can be local or systemic.

As used herein, the term “pharmaceutically acceptable” refers to a carrier that is compatible with the other ingredients of the formulation and not deleterious to the recipient thereof. The term is used synonymously with “physiologically acceptable” and “pharmacologically acceptable”. A pharmaceutical composition will generally comprise agents for buffering and preservation in storage, and can include buffers and carriers for appropriate delivery, depending on the route of administration. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The terms “dose” and “dosage” are used interchangeably herein. A dose refers to the amount of active ingredient given to an individual at each administration. For the present invention, the dose can refer to the concentration of the extracellular vesicles or associated components, e.g., the amount of therapeutic agent or dosage of radiolabel. The dose will vary depending on a number of factors, including frequency of administration; size and tolerance of the individual; severity of the condition; risk of side effects; the route of administration; and the imaging modality of the detectable moiety (if present). One of skill in the art will recognize that the dose can be modified depending on the above factors or based on therapeutic progress. The term “dosage form” refers to the particular format of the pharmaceutical and depends on the route of administration. For example, a dosage form can be in a liquid, e.g., a saline solution for injection.

“Subject,” “patient,” “individual” and like terms are used interchangeably and refer to, except where indicated, mammals such as humans and non-human primates, as well as rabbits, rats, mice, goats, pigs, and other mammalian species. The term does not necessarily indicate that the subject has been diagnosed with a particular disease, but typically refers to an individual under medical supervision. A patient can be an individual that is seeking treatment, monitoring, adjustment or modification of an existing therapeutic regimen, etc.

As used herein, the following meanings apply unless otherwise specified. The word “may” is used in a permissive sense (i.e., meaning having the potential to), rather than the mandatory sense (i.e., meaning must). The words “include”, “including”, and “includes” and the like mean including, but not limited to. The singular forms “a,” “an,” and “the” include plural referents. Thus, for example, reference to “an element” includes a combination of two or more elements, notwithstanding use of other terms and phrases for one or more elements, such as “one or more.” The term “or” is, unless indicated otherwise, non-exclusive, i.e., encompassing both “and” and “or.” The term “any of” between a modifier and a sequence means that the modifier modifies each member of the sequence. So, for example, the phrase “at least any of 1, 2 or 3” means “at least 1, at least 2 or at least 3”. The phrase “at least one” includes “a plurality”.

Definitions of common terms in cell biology and molecular biology can be found in “The Merck Manual of Diagnosis and Therapy”, 19th Edition, published by Merck Research Laboratories, 2006 (ISBN 0-91 1910-19-0); Robert S. Porter et al. (eds.), The Encyclopedia of Molecular Biology, published by Blackwell Science Ltd., 1994 (ISBN 0-632-02182-9); Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

The term “native form” corresponds to the polypeptide as it is understood to be encoded by the infectious agent's genome. The term “exosomal form” corresponds to any derivative of the protein that, in whole or in part, is fused to an exosome-associated protein. The term “cytoplasmic form” corresponds to any derivative of the protein that, in whole or in part, is configured, or designed, to be expressed within the cytoplasm of the cell, rather than entering the canonical secretory pathway.

The expression that a certain protein is “configured, or designed, to be expressed” in a certain way means that its nucleotide sequence encodes certain a particular amino acid sequence such that when that protein is expressed in a cell, that protein will be in its native form, exosomal form, or cytoplasmic form by virtue of that particular amino acid sequence. For instance, if a spike protein (S) is expressed in its native form, it is configured, or designed, to induce a humoral or cellular immune response by virtue of the fact that it is a transmembrane protein with an extracellular domain.

The term “extracellular vesicle” (EV) refers to lipid bilayer-delimited particles that are naturally released from cells. EVs range in diameter from around 20-30 nanometers to about 10 microns or more. EVs can comprise proteins, nucleic acids, lipids and metabolites from the cells that produced them. EVs include exosomes (about 50 to about 200 nm), microvesicles (about 100 to about 300 nm), ectosomes (about 50 to about 1000 nm), apoptotic bodies (about 50 to about 5000 nm) and lipid-protein aggregates of the same dimensions.

The term “nucleic acid” refers to polynucleotides such as deoxyribonucleic acid (DNA) or ribonucleic acid (RNA), as well as chemically modified nucleic acids such as morpholino (PMO), peptide nucleic acid (PNA), 2′O-methyl, 2′ methoxy-ethyl, phosphoramidate, methylphosphonate, and phosphorothioate. Nucleic acids may be of any size. Nucleic acids include but are not limited to genomic DNA, cDNA, mRNA, iRNA, miRNA, tRNA, ncRNA, rRNA, and recombinantly produced and chemically synthesized molecules such as aptamers, plasmids, anti-sense DNA strands, shRNA, ribozymes, nucleic acids conjugated and oligonucleotides. According to the invention, a nucleic acid may be present as a single-stranded or double-stranded and linear or covalently circularly closed molecule. A nucleic acid might be employed for introduction into, e.g., transfection of, cells, e.g., in the form of RNA which can be prepared by in vitro transcription from a DNA template. The RNA can moreover be modified before application by stabilizing sequences, capping, and polyadenylation. Generally, nucleic acid can be extracted, isolated, amplified, or analyzed by a variety of techniques such as those described by, e.g., Green and Sambrook, Molecular Cloning: A Laboratory Manual (Fourth Edition), Cold Spring Harbor Laboratory Press, Woodbury, NY 2,028 pages (2012).

The terms “peptide”, “polypeptide” and “protein” are used interchangeably herein and refer to any chain of at least two amino acids, linked by a covalent chemical bound. As used herein a peptide can refer to the complete amino acid sequence coding for an entire protein or to a portion thereof. A “protein coding sequence” or a sequence that “encodes” a particular polypeptide or peptide, is a nucleic acid sequence that is transcribed (in the case of DNA) and is translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the coding sequence.

As used herein, the phrase “protein polypeptide” means a polypeptide sequence of or derived from a protein. For example, a CD9 protein polypeptide may be any polypeptide of the CD9 protein, such as, e.g., a full length CD9 protein, a transmembrane domain polypeptide of a CD9 protein, a C-terminal stretch of a CD9 protein, an extracellular loop region of a CD9 protein, the intracellular (intralumenal) loop region of a CD9 protein, a C-terminal stretch of a CD9 protein, combinations thereof, and/or the like. Here, a protein polypeptide may be at least 10 amino acids long.

As used herein, the term “spike protein” includes any SARS-CoV-2 spike glycoprotein, fragment of a SARS-CoV-2 spike glycoprotein, monomer of a SARS-CoV-2 spike glycoprotein, trimer of SARS-CoV-2 spike glycoprotein monomers, variant of a SARS-CoV-2 spike glycoprotein, fusion protein or chimeral protein containing a SARS-CoV-2 spike glycoprotein sequence and another non-SARS-CoV-2 spike glycoprotein sequence, SARS-CoV-2 spike glycoproteins having one or more deletions, additions, or substitutions of one or more amino acids, and conservatively substituted variations of a SARS-CoV-2 spike glycoprotein having at least 80% amino acid sequence identity of e.g., at least the stem region of an S2 subunit, membrane-proximal stem helix region, or the receptor binding domain, or other like domains.

A fragment of a SARS-CoV-2 spike glycoprotein includes peptide or polypeptides the encompass, comprise, consist of, or overlap with e.g., antigenic epitopes, specific domains like receptor binding domain (RBD) in up or down conformational states, a receptor-binding fragment S1, fusion fragment S2, N-terminal domain (NTD), receptor-binding domain (RBD) C-terminal domain 1 (CTD1), C-terminal domain 2 (CTD2), fusion peptide (FP), fusion-peptide proximal region (FPPR), heptad repeat 1 (HR1), central helix (CH), connector domain (CD), heptad repeat 2 (HR2), transmembrane segment (TM), the cytoplasmic tail (CT), and the like.

A variant of a SARS-CoV-2 spike glycoprotein includes any known or yet to be discovered, including alpha, beta, gamma, delta, epsilon, eta, iota, kappa, 1.617.3, mu, zeta, omicron, or their subvariants, lineages, and conservatively substituted spike protein sequence.

SARS-CoV-2 spike glycoprotein may have additions, deletions, substitution, point mutations. For example, a spike protein may have a deletion of several (2-20) amino acids from its C-terminus (see Johnson et al., 2020 and Xiong et al., 2020), or furin cleavage site change (see e.g., Johnson et al., 2020).

As used herein, the term “nucleocapsid protein” means a soluble coronavirus structural protein that binds and forms complexes with RNA and viral membrane protein (M) and is critical for viral genome packaging. Nucleocapsid protein contains (from amino terminus to carboxy terminus) an intrinsically disordered region (IDR), and N terminal domain containing an RNA binding domain (NTD), a serine-arginine rich linker region (LKR), a C terminal domain containing an RNA binding and dimerization domain and a nuclear localization signal, followed by a C terminal IDR. Nucleocapsid protein is generally described, e.g., in McBride et al., “The Coronavirus Nucleocapsid Is a Multifunctional Protein,” Viruses. 2014 August; 6(8): 2991-3018; Cubuk et al., “The SARS-CoV-2 nucleocapsid protein is dynamic, disordered, and phase separates with RNA,” Nature Communications volume 12, Article number: 1936 (2021); and references cited therein.

Fusion proteins or chimeral proteins of SARS-CoV-2 spike glycoprotein or SARS-CoV-2 nucleocapsid protein includes fusions of spike or nucleocapsid protein sequences and sequences of another protein to effect a particular outcome, such as improved sorting or targeting to endosomes and resultant extracellular vesicles such as exosomes. For example, spike protein may be a fusion of spike or nucleocapsid protein sequences and other glycoproteins known to sort to exosomes or useful in the production of pseudovirions, such as VSV glycoproteins, lentivirus glycoprotein, and the like. Spike or nucleocapsid protein may be a fusion of spike protein sequences and other proteins known to sort to exosomes such as various tetraspanins (CD9, CD63, and CD81). Fusion proteins may contain mostly SARS-CoV-2 sequences with short (i.e., dipeptides to peptides of 100 amino acids) sequences of the other protein.

As used herein, the term “tetraspanin” or “tetraspanin protein” means any member (or chimera thereof) of a family of proteins having four transmembrane domains and in some cases present in exosome membranes. Tetraspanin proteins are known to regulate trafficking and cell and membrane compartmentalization. Tetraspanins include inter alia CD9, CD37, CD63, CD81, CD82, CD151, TSPAN7, TSPAN8, TSPAN12, TSPAN33, peripherin, UPla/lb, TSP-15, TSP-12, TSP3A, TSP86D, TSP26D, TSP-2, and analogs, orthologs, and homologs thereof. It is envisioned that useful tetraspanins may include chimeras of any one or more canonical tetraspanins, such as, e.g., a CD9/CD81 chimera or the like. Tetraspanins are generally described in Charrin et al., “Tetraspanins at a glance,” J Cell Sci (2014) 127 (17): 3641-3648; Kummer et al., “Tetraspanins: integrating cell surface receptors to functional microdomains in homeostasis and disease,” Med Microbiol Immunol. 2020; 209(4): 397-405; and references cited therein. The CD9 member of the tetraspanin superfamily is generally described in Umeda et al., “Structural insights into tetraspanin CD9 function,” Nature Communications volume 11, Article number: 1606 (2020), and references cited therein.

The term “sequence identity” as used herein refers to the percentage of sequence identity between two polypeptide sequences or two nucleic acid sequences. To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions times 100%). In one embodiment, the two sequences are the same length. The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present application. BLAST protein searches can be performed with the XBLAST program parameters set, e.g., to score-50, wordlength=3 to obtain amino acid sequences homologous to a protein molecule described herein. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website).

Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

For antibodies, percentage sequence identities can be determined when antibody sequences are maximally aligned by IMGT. After alignment, if a subject antibody region (e.g., the entire mature variable region of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, multiplied by 100 to convert to percentage.

Percent amino acid sequence identity may also be determined using the sequence comparison program NCBI-BLAST2 (Altschul et al., Nucleic Acids Res. 25:3389-3402 (1997)). The NCBI-BLAST2 sequence comparison program may be obtained from the National Institute of Health, Bethesda, Md. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62.

In situations where NCBI-BLAST2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

$$100 times the fraction X / Y ,$$

where X is the number of amino acid residues scored as identical matches by the sequence alignment program NCBI-BLAST2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. The term “nucleic acid sequence” as used herein refers to a sequence of nucleoside or nucleotide monomers consisting of naturally occurring bases, sugars and intersugar (backbone) linkages and includes cDNA. The term also includes modified or substituted sequences comprising non-naturally occurring monomers or portions thereof. The nucleic acid sequences of the present application may be deoxyribonucleic acid sequences (DNA) or ribonucleic acid sequences (RNA) and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine. It is understood that polynucleotides comprising non-transcribable nucleotide bases may be useful as probes in, for example, hybridization assays. The nucleic acid can be either double stranded or single stranded, and represents the sense or antisense strand. Further, the term “nucleic acid” includes the complementary nucleic acid sequences as well as codon optimized or synonymous codon equivalents.

As used herein the term “antibody” refers to immunoglobulin (Ig) molecules and immunologically active portions of immunoglobulin molecules, i.e., molecules that contain an antigen-binding site that specifically binds an antigen. Antibodies are usually heterotetrameric glycoproteins of about 150,000 Daltons, composed of two identical light (L) chains and two identical heavy (H) chains. The light chains from any vertebrate species can be assigned to one of two clearly distinct types, called kappa (κ) and lambda (λ), based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2. The heavy-chain constant domains that correspond to the different classes of immunoglobulins are called α, δ, ε, γ, and μ, respectively. The subunit structures and three-dimensional configurations of different classes of immunoglobulins are well known. The antibody may have one or more effector functions which refer to those biological activities attributable to the Fc region (a native sequence Fc region or amino acid sequence variant Fc region or any other modified Fc region) of an antibody. Non-limiting examples of antibody effector functions include C1q binding; complement dependent cytotoxicity; Fc receptor binding; antibody-dependent cell-mediated cytotoxicity (ADCC); phagocytosis; down regulation of cell surface receptors (e.g., B cell receptor (BCR); and cross-presentation of antigens by antigen presenting cells or dendritic cells).

The term “neutralizing antibody” (Nab) refers an antibody that defends a cell from a pathogen or infectious particle by neutralizing any effect it has biologically. Neutralization renders the particle no longer infectious or pathogenic. Neutralizing antibodies are part of the humoral response of the adaptive immune system against viruses, intracellular bacteria and microbial toxin. By binding specifically to surface antigen on an infectious particle, neutralizing antibodies prevent the particle from interacting with its host cells it might infect and destroy.

Immunity due to neutralizing antibodies is also known as sterilizing immunity, as the immune system eliminates the infectious particle before any infection took place.

The term “antigen” refers to any substance that will elicit an immune response. For instance, an antigen relates to any substance, preferably a peptide or protein, that reacts specifically with antibodies or T-lymphocytes (T cells). As used herein, the term “antigen” comprises any molecule which comprises at least one epitope. For instance, an antigen is a molecule which, optionally after processing, induces an immune reaction. For instance, any suitable antigen may be used, which is a candidate for an immune reaction, wherein the immune reaction may be a cellular immune reaction. For instance, the antigen may be presented by a cell, which results in an immune reaction against the antigen. For example, an antigen is a product which corresponds to or is derived from a naturally occurring antigen. Such antigens include, but are not limited to, SARS-CoV-2 structural proteins S, N, M, and E, and any variants or mutants thereof.

The term “pharmaceutical composition” refers to a formulation comprising an active ingredient, and optionally a pharmaceutically acceptable carrier, diluent or excipient. The term “active ingredient” can interchangeably refer to an “effective ingredient,” and is meant to refer to any agent that is capable of inducing a sought-after effect upon administration. By “pharmaceutically acceptable” it is meant the carrier, diluent or excipient must be compatible with the other ingredients of the formulation and not deleterious to the recipient thereof, nor to the activity of the active ingredient of the formulation. Pharmaceutically acceptable carriers, excipients or stabilizers are well known in the art, for example Remington's Pharmaceutical Sciences, 16th edition, Osol, A. Ed. (1980). Pharmaceutically acceptable carriers, excipients, or stabilizers are nontoxic to recipients at the dosages and concentrations employed, and may include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (for example, Zn-protein complexes); and/or non-ionic surfactants such as TWEEN™, PLURONICS™ or polyethylene glycol (PEG). Examples of carrier include, but are not limited to, liposome, nanoparticles, ointment, micelles, microsphere, microparticle, cream, emulsion, and gel.

Examples of excipient include, but are not limited to, anti-adherents such as magnesium stearate, binders such as saccharides and their derivatives (sucrose, lactose, starches, cellulose, sugar alcohols and the like) protein like gelatin and synthetic polymers, lubricants such as talc and silica, and preservatives such as antioxidants, vitamin A, vitamin E, vitamin C, retinyl palmitate, selenium, cysteine, methionine, citric acid, sodium sulfate and parabens. Examples of diluent include, but are not limited to, water, alcohol, saline solution, glycol, mineral oil and dimethyl sulfoxide (DMSO).

The term “vaccine” or “immunogenic composition” relates to a pharmaceutical preparation (pharmaceutical composition) or product that upon administration induces an immune response, e.g., a cellular immune response, which recognizes and attacks a pathogen or a diseased cell. The term “immune response” refers to an integrated bodily response to an antigen and refers to a cellular immune response and/or a humoral immune response. The immune response may be protective/preventive/prophylactic and/or therapeutic.

The term “cellular immune response” or “cell-mediated immune response” describes any adaptive immune response in which antigen-specific T cells have the main role. It is defined operationally as all adaptive immunity that cannot be transferred to a native recipient by serum antibody. In contrast, the term “humoral immune response” describes immunity due to antibodies.

The cellular response relates to cells called T cells or T-lymphocytes which act as either “helpers” or “killers”. The helper T cells (also termed CD4+ T cells) play a central role by regulating the immune response and the killer cells (also termed cytotoxic T cells, cytolytic T cells, CD8+ T cells or CTLs) kill diseased cells such as cancer cells, preventing the production of more diseased cells.

The terms “immunoreactive cell” “immune cells” or “immune effector cells” relate to a cell which exerts effector functions during an immune reaction. An “immunoreactive cell” preferably is capable of binding an antigen or a cell characterized by presentation of an antigen, or an antigen peptide derived from an antigen and mediating an immune response. For example, such cells secrete cytokines and/or chemokines, secrete antibodies, recognize cancerous cells, and optionally eliminate such cells. For example, immunoreactive cells comprise T cells (cytotoxic T cells, helper T cells, tumor infiltrating T cells), B cells, natural killer cells, neutrophils, macrophages, and dendritic cells.

The term “adjuvant” refers to a pharmacological or immunological agent that modifies the effect of other agents. An adjuvant may be added to the vaccine composition of the invention to boost the immune response to produce more antibodies and longer-lasting immunity, thus minimizing the dose of antigen needed. Adjuvants may also be used to enhance the efficacy of a vaccine by helping to modify the immune response to particular types of immune system cells: for example, by activating T cells instead of antibody-secreting B cells depending on the purpose of the vaccine. Immunologic adjuvants are added to vaccines to stimulate the immune system's response to the target antigen, but do not provide immunity themselves. Examples of adjuvants include, but are not limited to analgesic adjuvants; inorganic compounds such as alum, aluminum hydroxide, aluminum phosphate, calcium phosphate hydroxide; mineral oil such as paraffin oil; bacterial products such as killed bacteria (Bordetella pertussis, Mycobacterium bovis, toxoids); nonbacterial organics such as squalene; delivery systems such as detergents (Quil A); plant saponins from Quillaja, soybean, or Polygala senega; cytokines such as IL-1, IL-2, IL-12; combination such as Freund's complete adjuvant, Freund's incomplete adjuvant; food-based oil such as Adjuvant 65, which is based on peanut oil.

### DETAILED EMBODIMENTS

Embodiment 1. In one embodiment, an immunogenic composition comprising a vesicle and a fusion protein is provided, wherein said fusion protein comprises a virus polypeptide and an exosomal polypeptide, is provided.

Embodiment 2. An immunogenic composition of embodiment 1 further comprising an excipient is provided, is provided.

Embodiment 3. An immunogenic composition of embodiment 2, wherein the excipient is a buffer, is provided.

Embodiments 4. An immunogenic composition of any one of embodiments 1-3 not comprising an adjuvant is provided, is provided.

Embodiment 5. An immunogenic composition of any one of embodiments 1-4, wherein said fusion protein is present in a membrane of a vesicle, is provided.

Embodiment 6. An immunogenic composition of any one of embodiments 1-5, wherein a part or all of the virus polypeptide is present at or on an outer surface of A vesicle, is provided.

Embodiment 7. An immunogenic composition of any one of embodiments 1-6, wherein the fusion protein is present in the composition at a concentration of about 1 ng/100 μL to about 150 ng/100 μL, is provided.

Embodiment 8. An immunogenic composition of any one of embodiments 1-7, wherein the exosomal polypeptide is a tetraspanin protein polypeptide, is provided.

Embodiment 9. An immunogenic composition of any one of embodiments 1-8, wherein the exosomal polypeptide is a CD9 protein polypeptide, is provided.

Embodiment 10. An immunogenic composition of any one of embodiments 1-9, wherein the exosomal polypeptide comprises an amino acid sequence having at least 80% identity to SEQ ID NO:10, is provided.

Embodiment 11. An immunogenic composition of any one of embodiments 1-10, wherein the exosomal polypeptide comprises an amino acid sequence of SEQ ID NO:10, is provided.

Embodiment 12. An immunogenic composition of any one of embodiments 1-11, wherein the virus polypeptide is a SARS-CoV-2 polypeptide, is provided.

Embodiment 13. An immunogenic composition of any one of embodiments 1-12, wherein the virus polypeptide is a SARS-CoV-2 spike protein polypeptide, is provided.

Embodiment 14. An immunogenic composition of any one of embodiments 1-13, wherein the virus polypeptide is a SARS-CoV-2 delta variant spike protein polypeptide, is provided.

Embodiment 15. An immunogenic composition of any one of embodiments 1-14, wherein the virus polypeptide comprises an amino acid sequence having at least 80% identity to SEQ ID NO:1, is provided.

Embodiment 16. An immunogenic composition of any one of embodiments 1-15, wherein the virus polypeptide comprises an amino acid sequence of SEQ ID NO:1, is provided.

Embodiment 17. An immunogenic composition of any one of embodiments 1-11, wherein the virus polypeptide is a SARS-CoV-2 nucleocapsid protein polypeptide, is provided.

Embodiment 18. An immunogenic composition of any one of embodiments 1-11 and 17, wherein the virus polypeptide comprises an amino acid sequence having at least 80% identity to SEQ ID NO:5, is provided.

Embodiment 19. An immunogenic composition of any one of embodiments 1-11, 17 and 18, wherein the virus polypeptide comprises an amino acid sequence of SEQ ID NO:5, is provided.

Embodiment 20. An immunogenic composition of any one of embodiments 1-16, wherein the fusion protein comprises a SARS-CoV-2 spike protein polypeptide and a CD9 protein polypeptide, is provided.

Embodiment 21. An immunogenic composition of any one of embodiments 1-16 and 20, wherein the fusion protein comprises an amino acid sequence having at least 80% identity to SEQ ID NO:2, is provided.

Embodiment 22. An immunogenic composition of any one of embodiments 1-16, 20 and 21, wherein the fusion protein comprises an amino acid sequence having a sequence of SEQ ID NO:2, is provided.

Embodiment 23. An immunogenic composition of any one of embodiments 1-16 and 20-22, wherein the fusion protein comprises an amino acid sequence having at least 80% identity to SEQ ID NO:3, is provided.

Embodiment 24. An immunogenic composition of any one of embodiments 1-16, and 20-23, wherein the fusion protein comprises an amino acid sequence having a sequence of SEQ ID NO:3, is provided.

Embodiment 25. An immunogenic composition of any one of embodiments 1-11 and 17-19, wherein the fusion protein comprises a SARS-CoV-2 nucleocapsid protein polypeptide and a CD9 protein polypeptide, is provided.

Embodiment 26. An immunogenic composition of any one of embodiments 1-11, 17-19 and 25, wherein the fusion protein comprises an amino acid sequence having at least 80% identity to SEQ ID NO:6, is provided.

Embodiment 27. An immunogenic composition of any one of embodiments 1-11, 17-19, 25 and 26, wherein the fusion protein comprises an amino acid sequence having a sequence of SEQ ID NO:6, is provided.

Embodiment 28. An immunogenic composition of any one of embodiments 1-11, 17-19, and 25-27, wherein the fusion protein comprises an amino acid sequence having at least 80% identity to SEQ ID NO:7, is provided.

Embodiment 29. An immunogenic composition of any one of embodiments 1-11, 17-19, and 25-28, wherein the fusion protein comprises an amino acid sequence having a sequence of SEQ ID NO:7, is provided.

Embodiment 30. An immunogenic composition of any one of embodiments 1-29, wherein an immunogenic dose of said composition comprises (i) about 1 ng to about 300 ng of fusion protein or virus polypeptide or (ii) 10-200 ng total fusion protein in 0.5 mL, is provided.

Embodiment 31. An immunogenic composition of any one of embodiments 1-30 comprising a second vesicle and a second fusion protein, wherein said second fusion protein comprises a second virus polypeptide and an exosomal polypeptide, and wherein said second fusion protein is present in a membrane of the second vesicle, is provided.

Embodiment 32. An immunogenic composition of embodiment 31, wherein said virus polypeptide is a SARS-CoV-2 spike protein polypeptide and said second virus polypeptide is a SARS-CoV-2 nucleocapsid protein, is provided.

Embodiment 33. An immunogenic composition of embodiment 31 or 32, wherein the fusion protein comprises an amino acid sequence having at least 80% identity to SEQ ID NO:2, and wherein the second fusion protein comprises an amino acid sequence having at least 80% identity to SEQ ID NO:6, is provided.

Embodiment 34. An immunogenic composition of any one of embodiments 31-33, wherein the fusion protein comprises an amino acid sequence of SEQ ID NO:2, and wherein the second fusion protein comprises an amino acid sequence of SEQ ID NO:6, is provided.

Embodiment 35. An immunogenic composition of any one of embodiments 31-34, wherein the fusion protein comprises an amino acid sequence having at least 80% identity to SEQ ID NO:3, and wherein the second fusion protein comprises an amino acid sequence having at least 80% identity to SEQ ID NO:7, is provided.

Embodiment 36. An immunogenic composition of any one of embodiments 31-35, wherein the fusion protein comprises an amino acid sequence of SEQ ID NO:3, and wherein the second fusion protein comprises an amino acid sequence of SEQ ID NO:7, is provided.

Embodiment 37. An immunogenic composition of any one of embodiments 1-36, wherein A vesicle is an exosome, is provided.

Embodiment 38. A method of immunizing a subject against a virus infection comprising administering to the subject an immunogenically effective dose of an immunogenic composition of any one of embodiments 1-37, is provided.

Embodiment 39. A method of embodiment 38, wherein said immunogenically effective dose elicits protective immunity in said subject against more than one variant of a given virus, is provided.

Embodiment 40. A method of embodiment 38 or 39, wherein An immunogenic composition comprises a SARS-CoV-2 nucleocapsid protein polypeptide, is provided.

Embodiment 41. A method of any one of embodiments 38-40, wherein An immunogenic composition comprises a fusion protein having an amino acid sequence that is at least 80% identical to SEQ ID NO:6, is provided.

Embodiment 42. A method of any one of embodiments 38-41, wherein An immunogenic composition comprises a fusion protein having an amino acid sequence that is at least 80% identical to SEQ ID NO:7, is provided.

Embodiment 43. A method of any one of embodiments 38-42, wherein An immunogenic composition comprises a fusion protein having an amino acid sequence of SEQ ID NO:6, is provided.

Embodiment 44. A method of any one of embodiments 38-43, wherein An immunogenic composition comprises a fusion protein having an amino acid sequence of SEQ ID NO:7, is provided.

Embodiment 45. A method of embodiment 38 or 39, wherein An immunogenic composition comprises a SARS-CoV-2 delta variant spike protein polypeptide and An immunogenically effective dose elicits protective immunity in said subject against a SARS-CoV-2 delta variant and a SARS-CoV-2 omicron variant, is provided.

Embodiment 46. A method of any one of embodiments 38, 39, and 45, wherein An immunogenic composition comprises a fusion protein having an amino acid sequence that is at least 80% identical to SEQ ID NO:2, is provided.

Embodiment 47. A method of any one of embodiments 38, 39, 45 and 46, wherein An immunogenic composition comprises a fusion protein having an amino acid sequence that is at least 80% identical to SEQ ID NO:3, is provided.

Embodiment 48. A method of any one of embodiments 38, 39, and 45-47, wherein An immunogenic composition comprises a fusion protein having an amino acid sequence of SEQ ID NO:2, is provided.

Embodiment 49. A method of any one of embodiments 38, 39, and 45-48, wherein An immunogenic composition comprises a fusion protein having an amino acid sequence of SEQ ID NO:3, is provided.

Embodiment 50. A method of embodiment 38 or 39, wherein An immunogenic composition comprises a first fusion protein comprising a SARS-CoV-2 nucleocapsid protein polypeptide located in the membrane of a first vesicle and a second fusion protein comprising a SARS-CoV-2 spike protein polypeptide located in the membrane of a second vesicle, is provided.

Embodiment 51. A method of embodiment 50, wherein the first fusion protein has an amino acid sequence that is at least 80% identical to SEQ ID NO:6, is provided.

Embodiment 52. A method of embodiment 50 or 51, wherein the first fusion protein has an amino acid sequence that is at least 80% identical to SEQ ID NO:7, is provided.

Embodiment 53. A method of any one of embodiments 50-52, wherein the first fusion protein has an amino acid sequence of SEQ ID NO:6, is provided.

Embodiment 54. A method of any one of embodiments 50-53, wherein the first fusion protein has an amino acid sequence of SEQ ID NO:7, is provided.

Embodiment 55. A method of any one of embodiments 50-54, wherein the second fusion protein has an amino acid sequence that is at least 80% identical to SEQ ID NO:2, is provided.

Embodiment 56. A method of any one of embodiments 50-55, wherein the second fusion protein has an amino acid sequence that is at least 80% identical to SEQ ID NO:3, is provided.

Embodiment 57. A method of any one of embodiments 50-56, wherein the second fusion protein has an amino acid sequence of SEQ ID NO:2, is provided.

Embodiment 58. A method of any one of embodiments 50-57, wherein the second fusion protein has an amino acid sequence of SEQ ID NO:3, is provided.

Embodiment 59. A method of any one of embodiments 38-58, wherein An immunogenically effective dose comprises (i) about 1 ng to about 300 ng of fusion protein or virus polypeptide or (ii) 10-200 ng total fusion protein in 0.5 mL, is provided.

Embodiment 60. A method of any one of embodiments 38-59 further comprising administering to the subject a second effective dose of An immunogenic composition of any one of embodiments 1-37, is provided.

Embodiment 61. A synthetic fusion protein comprising a virus polypeptide and an exosomal polypeptide, is provided.

Embodiment 62. A synthetic fusion protein of embodiment 61 further comprising a linker polypeptide positioned between the virus polypeptide and the exosomal polypeptide, is provided.

Embodiment 63. A synthetic fusion protein of embodiment 61 or 62 further comprising a hinge polypeptide positioned between the virus polypeptide and the exosomal polypeptide, is provided.

Embodiment 64. A synthetic fusion protein of any one of embodiments 61-63 further comprising a transmembrane domain polypeptide positioned between the virus polypeptide and the exosomal polypeptide, is provided.

Embodiment 65. A synthetic fusion protein of any one of embodiments 61-64, wherein the exosomal polypeptide is a tetraspanin polypeptide, is provided.

Embodiment 66. A synthetic fusion protein of any one of embodiments 61-65, wherein the exosomal polypeptide is a CD9 polypeptide, is provided.

Embodiment 67. A synthetic fusion protein of any one of embodiments 61-66, wherein the virus polypeptide is a SARS-CoV-2 structural protein polynucleotide, is provided.

Embodiment 68. A synthetic fusion protein of any one of embodiments 61-67, wherein the virus polypeptide is a SARS-CoV-2 spike protein polypeptide, is provided.

Embodiment 69. A synthetic fusion protein of embodiment 68, wherein said SARS-CoV-2 spike protein polypeptide comprises a one of more of a furin cleavage site mutation (CSM [682RRAR685-to-682GSAG685]) and a di-proline substitution (2P [986KV987-to-986PP987]), is provided.

Embodiment 70. A synthetic fusion protein of any one of embodiments 61-69, wherein the fusion protein comprises, in order from amino terminus to carboxy terminus, a SARS-CoV-2 spike protein polypeptide, a linker polypeptide, and a CD9 polypeptide, is provided.

Embodiment 71. A synthetic fusion protein of any one of embodiments 61-70, wherein the fusion protein comprises an amino acid sequence that is at least 80% identical to SEQ ID NO:3, is provided.

Embodiment 72. A synthetic fusion protein of any one of embodiments 61-71, wherein the fusion protein comprises an amino acid sequence set forth in SEQ ID NO:3, is provided.

Embodiment 73. A synthetic fusion protein of any one of embodiments 61-67, wherein the virus polypeptide in a SARS-CoV-2 nucleocapsid protein polypeptide, is provided.

Embodiment 74. A synthetic fusion protein of any one of embodiments 61-67 and 73, wherein the fusion protein comprises, in order from amino terminus to carboxy terminus, a signal peptide, a SARS-CoV-2 nucleocapsid protein polypeptide, a hinge region, a transmembrane domain polypeptide, a linker polypeptide, and a CD9 polypeptide, is provided.

Embodiment 75. A synthetic fusion protein of any one of embodiments 61-67, 73 and 74, wherein the fusion protein comprises an amino acid sequence that is at least 80% identical to SEQ ID NO:7, is provided.

Embodiment 76. A synthetic fusion protein of any one of embodiments 61-67 and 73-75, wherein the fusion protein comprises an amino acid sequence set forth in SEQ ID NO:7, is provided.

Embodiment 77. A synthetic polynucleotide encoding a synthetic fusion protein of any one of embodiments 61-76, is provided.

Embodiment 78. A synthetic polynucleotide of embodiment 77 comprising a nucleic acid sequence having at least 80% identity to SEQ ID NO:13, is provided.

Embodiment 79. A synthetic polynucleotide of embodiment 77 or 78 comprising a nucleic acid sequence set forth in SEQ ID NO:13, is provided.

Embodiment 80. A synthetic polynucleotide of any one of embodiments 77-79 comprising a nucleic acid sequence having at least 80% identity to SEQ ID NO:11, is provided.

Embodiment 81. A synthetic polynucleotide of any one of embodiments 77-80 comprising a nucleic acid sequence set forth in SEQ ID NO:11, is provided.

Embodiment 82. A synthetic polynucleotide of any one of embodiments 77-81 comprising a nucleic acid sequence having at least 80% identity to SEQ ID NO:4, is provided.

Embodiment 83. A synthetic polynucleotide of any one of embodiments 77-82 comprising a nucleic acid sequence set forth in SEQ ID NO:4, is provided.

Embodiment 84. A synthetic polynucleotide of any one of embodiments 77-79 comprising a nucleic acid sequence having at least 80% identity to SEQ ID NO:12, is provided.

Embodiment 85. A synthetic polynucleotide of any one of embodiments 77-79 and 84 comprising a nucleic acid sequence set forth in SEQ ID NO:12, is provided.

Embodiment 86. A synthetic polynucleotide of any one of embodiments 77-79, 84 and 85 comprising a nucleic acid sequence having at least 80% identity to SEQ ID NO:8, is provided.

Embodiment 87. A synthetic polynucleotide of any one of embodiments 77-79 and 84-86 comprising a nucleic acid sequence set forth in SEQ ID NO:8, is provided.

Embodiment 88. A cell comprising a synthetic polynucleotide of any one of embodiments 77-87, is provided.

Embodiment 89. A cell of embodiment 88, wherein A cell is a metazoan cell, is provided.

Embodiment 90. A cell of embodiment 88 or 89 wherein A cell is a vertebrate cell, is provided.

Embodiment 91. A cell of any one of embodiments 88-90, wherein A cell is a mammalian cell, is provided.

Embodiment 92. A cell of any one of embodiments 88-91, wherein A cell is a primate cell, is provided.

Embodiment 93. A cell of any one of embodiments 88-92, wherein A cell is a human cell, is provided.

Embodiment 94. A cell of any one of embodiments 88-93, wherein A cell is a primary cell, is provided.

Embodiment 95. A cell of any one of embodiments 88-93, wherein A cell is a human embryonic kidney cell, is provided.

Embodiment 96. A cell of embodiment 95, wherein A cell is a 293 cell, is provided.

Embodiment 97. A cell of any one of embodiments 88-96, wherein said cell is produced by transducing A cell with a lentivirus comprising A synthetic polynucleotide, is provided.

Embodiment 98. A cell of any one of embodiments 88-97, wherein said cell comprises a synthetic fusion protein of any one of embodiments 61-77, is provided.

Embodiment 99. A vesicle comprising a synthetic fusion protein of any one of embodiments 61-76, is provided.

Embodiment 100. A vesicle of embodiment 99, wherein said vesicle is an exosome, is provided.

Embodiment 101. A vesicle of embodiment 99 or 100, wherein said vesicle has a diameter of about 50-500 nm, is provided.

Embodiment 102. A vesicle of any one of embodiments 99-101, wherein a SARS-CoV-2 spike protein polypeptide is expressed on an outer surface of said vesicle, is provided.

Embodiment 103. A vesicle of any one of embodiments 99-102, wherein said vesicle expresses a SARS-CoV-2 nucleocapsid protein polypeptide on its surface, is provided.

Embodiment 104. A method of making a vesicle of any one of embodiments 99-103 comprising culturing a cell of any one of embodiments 88-98 in a cell culture medium, collecting a cell culture medium, and purifying a plurality of vesicles comprising said vesicle from a cell culture medium, is provided.

Embodiment 105. A method of embodiment 104 further comprising inducing expression of a synthetic polynucleotide of any one of embodiments 77-87 to produce a synthetic fusion protein of any one of embodiments 61-76, is provided.

Embodiment 106. A method of embodiment 105, wherein said inducing comprises contacting A cell with tetracycline, doxycycline, or analogs thereof.

Embodiment 107. A method of embodiment 105, wherein said inducing comprises removing from A cell tetracycline, doxycycline, or analogs thereof.

Embodiment 108. A method of eliciting an immune response in a subject comprising administering to said subject a first dose of an immunogenic composition comprising a plurality of vesicles comprising a vesicle of any one of embodiments 99-103 or a vesicle produced according to a method of any one of embodiments 104-107, wherein a synthetic fusion protein of any one of embodiments 61-76 is expressed on an outer surface of said vesicle, is provided.

Embodiment 109. A method of embodiment 108 further comprising administering to said subject a second dose of An immunogenic composition a period of time after administering the first dose, is provided.

Embodiment 110. A method of embodiment 109, wherein the period of time is 14 days-1 year, is provided.

Embodiment 111. A method of any one of embodiments 108-110, wherein the first dose or the second dose comprises about 100 μL-1 mL of An immunogenic composition, wherein said immunogenic composition comprises about 0.3 ng/mL-3 μg/mL of said synthetic fusion protein, is provided.

Embodiment 112. A method of any one of embodiments 108-111, wherein the first dose or the second dose comprises about 100 μL-1 mL of An immunogenic composition which comprises about 2E9 vesicles/mL-3E13 vesicles/mL, is provided.

Embodiment 113. A method of any one of embodiments 108-112, wherein said synthetic fusion protein comprises a SARS-CoV-2 spike protein polypeptide, is provided.

Embodiment 114. A method of any one of embodiments 108-113, wherein said synthetic fusion protein comprises a SARS-CoV-2 nucleocapsid protein polypeptide, is provided.

Embodiment 115. A method of any one of embodiments 108-114, wherein said immunogenic composition comprises vesicles that express on the outer surface SARS-CoV-2 spike protein polypeptides, and other vesicles that express on the outer surface SARS-CoV-2 nucleocapsid protein polypeptides, is provided.

Embodiment 116. A method of any one of embodiments 108-115, wherein the elicited immune response comprises producing neutralizing antibodies against an antigen present in A synthetic fusion protein, is provided.

Embodiment 117. A method of any one of embodiments 108-116, wherein the elicited immune response comprises producing neutralizing antibodies against two or more SARS-CoV-2 variants, is provided.

Embodiment 118. A method of any one of embodiments 108-117, wherein the elicited immune response comprises producing anti-spike antibodies, is provided.

Embodiment 119. A method of any one of embodiments 108-118, wherein the elicited immune response comprises a spike-specific T cell response, is provided.

Embodiment 120. A method of any one of embodiments 108-119, wherein the elicited immune response comprises producing anti-nucleocapsid antibodies, is provided.

Embodiment 121. A method of any one of embodiments 108-120, wherein the elicited immune response comprises a nucleocapsid-specific T cell response, is provided.

Embodiment 122. A method of any one of embodiments 108-121, wherein the immune response persists in the subject for up to nine months, is provided.

Embodiment 123. A method of any one of embodiments 108-121, wherein the immune response persists in the subject for at least nine months, is provided.

### Sequences

SEQ ID NO: 1 provides an amino acid sequence of a coronavirus Spike protein
polypeptide.
MFVFLVLLPLVSSQCVNLRTRTQLPPAYTNSFTRGVYYPDKVFRSS
VLHSTQDLFLPFFSNVTWFHAIHVSGTNGTKRFDNPVLPFNDGVYF
ASTEKSNIIRGWIFGTTLDSKTQSLLIVNNATNVVIKVCEFQFCNDP
FLDVYYHKNNKSWMESGVYSSANNCTFEYVSQPFLMDLEGKQGN
FKNLREFVFKNIDGYFKIYSKHTPINLVRDLPQGFSALEPLVDLPIGI
NITRFQTLLALHRSYLTPGDSSSGWTAGAAAYYVGYLQPRTFLLK
YNENGTITDAVDCALDPLSETKCTLKSFTVEKGIYQTSNFRVQPTE
SIVRFPNITNLCPFGEVENATRFASVYAWNRKRISNCVADYSVLYN
SASFSTFKCYGVSPTKLNDLCFTNVYADSFVIRGDEVRQIAPGQTG
KIADYNYKLPDDFTGCVIAWNSNNLDSKVGGNYNYRYRLFRKSN
LKPFERDISTEIYQAGSKPCNGVEGENCYFPLQSYGFQPTNGVGYQ
PYRVVVLSFELLHAPATVCGPKKSTNLVKNKCVNFNFNGLTGTGV
LTESNKKFLPFQQFGRDIADTTDAVRDPQTLEILDITPCSFGGVSVIT
PGTNTSNQVAVLYQGVNCTEVPVAIHADQLTPTWRVYSTGSNVF
QTRAGCLIGAEHVNNSYECDIPIGAGICASYQTQTNSRGSAGSVAS
QSIIAYTMSLGAENSVAYSNNSIAIPTNFTISVTTEILPVSMTKTSVD
CTMYICGDSTECSNLLLQYGSFCTQLNRALTGIAVEQDKNTQEVF
AQVKQIYKTPPIKDFGGFNFSQILPDPSKPSKRSFIEDLLFNKVTLAD
AGFIKQYGDCLGDIAARDLICAQKFNGLTVLPPLLTDEMIAQYTSA
LLAGTITSGWTFGAGAALQIPFAMQMAYRENGIGVTQNVLYENQK
LIANQFNSAIGKIQDSLSSTASALGKLQNVVNQNAQALNTLVKQLS
SNFGAISSVLNDILSRLDPPEAEVQIDRLITGRLQSLQTYVTQQLIRA
AEIRASANLAATKMSECVLGQSKRVDFCGKGYHLMSFPQSAPHG
VVFLHVTYVPAQEKNFTTAPAICHDGKAHFPREGVFVSNGTHWFV
TQRNFYEPQIITTDNTFVSGNCDVVIGIVNNTVYDPLQPELDSFKEE
LDKYFKNHTSPDVDLGDISGINASVVNIQKEIDRLNEVAKNLNESLI
DLQELGKYEQYIKWPWYIWLGFIAGLIAIVMVTIMLCCMTSCCSCL
KGCCSCGSCCKF
SEQ ID NO: 2 provides an amino acid sequence of a Spike polypeptide-linker-CD9
proximal region chimera.
NESLIDLQELGKYEQYIKWPWYIWLGFIAGLIAIVMVTIMLCCMTS
CCSCLKGCCSCGSCCKFGGGGSGGGGSGGGGSPVKGGTKCIKYLL
FGFNFIFWLAGIAVLAIGLWLRFDSQTKSIFEQETNNNNSSFYTGVY
ILIGAGALMMLVGFLGCCGAVQESQCMLGL
SEQ ID NO: 3 provides an amino acid sequence of a Spike-CD9 fusion protein.
MFVFLVLLPLVSSQCVNLRTRTQLPPAYTNSFTRGVYYPDKVFRSS
VLHSTQDLFLPFFSNVTWFHAIHVSGTNGTKRFDNPVLPFNDGVYF
ASTEKSNIIRGWIFGTTLDSKTQSLLIVNNATNVVIKVCEFQFCNDP
FLDVYYHKNNKSWMESGVYSSANNCTFEYVSQPFLMDLEGKQGN
FKNLREFVFKNIDGYFKIYSKHTPINLVRDLPQGFSALEPLVDLPIGI
NITRFQTLLALHRSYLTPGDSSSGWTAGAAAYYVGYLQPRTFLLK
YNENGTITDAVDCALDPLSETKCTLKSFTVEKGIYQTSNFRVQPTE
SIVRFPNITNLCPFGEVFNATRFASVYAWNRKRISNCVADYSVLYN
SASFSTFKCYGVSPTKLNDLCFTNVYADSFVIRGDEVRQIAPGQTG
KIADYNYKLPDDFTGCVIAWNSNNLDSKVGGNYNYRYRLFRKSN
LKPFERDISTEIYQAGSKPCNGVEGENCYFPLQSYGFQPTNGVGYQ
PYRVVVLSFELLHAPATVCGPKKSTNLVKNKCVNFNFNGLTGTGV
LTESNKKFLPFQQFGRDIADTTDAVRDPQTLEILDITPCSFGGVSVIT
PGTNTSNQVAVLYQGVNCTEVPVAIHADQLTPTWRVYSTGSNVF
QTRAGCLIGAEHVNNSYECDIPIGAGICASYQTQTNSRGSAGSVAS
QSIIAYTMSLGAENSVAYSNNSIAIPTNFTISVTTEILPVSMTKTSVD
CTMYICGDSTECSNLLLQYGSFCTQLNRALTGIAVEQDKNTQEVF
AQVKQIYKTPPIKDFGGFNFSQILPDPSKPSKRSFIEDLLFNKVTLAD
AGFIKQYGDCLGDIAARDLICAQKFNGLTVLPPLLTDEMIAQYTSA
LLAGTITSGWTFGAGAALQIPFAMQMAYRENGIGVTQNVLYENQK
LIANQFNSAIGKIQDSLSSTASALGKLQNVVNQNAQALNTLVKQLS
SNFGAISSVLNDILSRLDPPEAEVQIDRLITGRLQSLQTYVTQQLIRA
AEIRASANLAATKMSECVLGQSKRVDFCGKGYHLMSFPQSAPHG
VVFLHVTYVPAQEKNFTTAPAICHDGKAHFPREGVFVSNGTHWFV
TQRNFYEPQIITTDNTFVSGNCDVVIGIVNNTVYDPLQPELDSFKEE
LDKYFKNHTSPDVDLGDISGINASVVNIQKEIDRLNEVAKNLNESLI
DLQELGKYEQYIKWPWYIWLGFIAGLIAIVMVTIMLCCMTSCCSCL
KGCCSCGSCCKFGGGGSGGGGSGGGGSPVKGGTKCIKYLLFGFNF
IFWLAGIAVLAIGLWLRFDSQTKSIFEQETNNNNSSFYTGVYILIGA
GALMMLVGFLGCCGAVQESQCMLGLFFGFLLVIFAIEIAAAIWGY
SHKDEVIKEVQEFYKDTYNKLKTKDEPQRETLKAIHYALNCCGLA
GGVEQFISDICPKKDVLETFTVKSCPDAIKEVFDNKFHIIGAVGIGIA
VVMIFGMIFSMILCCAIRRNREMV
SEQ ID NO: 4 provides a nucleic acid sequence encoding a Spike-CD9 fusion protein.
ATGTTTGTATTTCTGGTACTTTTGCCACTCGTCAGTTCCCAATGT
GTTAACCTGCGCACTCGTACGCAACTCCCCCCAGCATATACCAA
CTCATTTACGCGGGGAGTATATTATCCGGACAAGGTATTTAGGA
GTTCTGTACTGCACAGCACACAGGACCTTTTTCTTCCTTTTTTTT
CCAACGTGACATGGTTTCATGCGATACACGTAAGTGGAACCAA
TGGAACTAAAAGGTTCGATAATCCTGTACTTCCGTTTAACGATG
GTGTCTATTTCGCTTCTACGGAGAAGTCAAATATCATTAGAGGT
TGGATCTTCGGTACAACTCTTGACAGTAAGACGCAAAGTCTGCT
CATCGTAAACAACGCGACGAACGTAGTGATAAAGGTCTGCGAG
TTTCAGTTCTGTAATGATCCGTTTCTTGACGTCTATTACCATAAG
AACAATAAGAGTTGGATGGAAAGTGGAGTATACAGCAGCGCTA
ACAATTGCACATTTGAATATGTCTCTCAACCTTTCCTGATGGAC
CTCGAAGGGAAACAAGGTAACTTCAAAAATCTCCGAGAATTTG
TTTTTAAAAATATAGACGGATATTTCAAGATATACTCAAAGCAC
ACGCCGATAAATCTGGTCAGGGATCTCCCTCAGGGTTTCAGTGC
ACTCGAACCTTTGGTAGATTTGCCTATCGGAATAAATATTACAC
GCTTTCAGACGCTTCTTGCGTTGCACCGGTCTTACCTTACACCTG
GGGACAGTTCATCTGGTTGGACGGCCGGGGCTGCCGCTTATTAC
GTCGGATATCTCCAACCAAGAACGTTTCTGCTTAAATATAATGA
GAACGGCACAATAACTGACGCGGTGGATTGCGCTCTTGATCCTC
TCTCAGAGACCAAATGCACGCTCAAATCCTTTACTGTCGAGAAA
GGGATTTATCAGACCAGCAACTTTCGCGTGCAGCCTACAGAAA
GTATTGTAAGATTCCCTAACATTACTAACCTCTGCCCGTTCGGC
GAGGTATTTAATGCAACACGATTTGCAAGTGTCTATGCGTGGAA
TAGAAAACGCATTTCAAATTGTGTCGCCGATTACTCCGTACTCT
ATAACAGCGCCTCATTTAGCACGTTTAAATGTTATGGTGTCTCC
CCCACAAAGCTCAATGATCTCTGCTTCACTAACGTGTATGCTGA
TAGCTTCGTGATCCGAGGTGACGAAGTGAGGCAAATTGCTCCG
GGTCAAACCGGGAAGATCGCAGATTATAACTATAAGTTGCCAG
ACGATTTCACTGGATGTGTTATTGCGTGGAACTCTAATAACCTG
GACTCAAAAGTGGGAGGGAATTACAATTACCGCTACAGGCTGT
TTCGCAAAAGCAATTTGAAACCCTTCGAGAGGGACATCAGTAC
TGAGATATACCAGGCAGGGTCAAAACCCTGCAATGGAGTCGAA
GGATTTAATTGCTACTTCCCGCTTCAGAGCTATGGGTTCCAGCC
AACGAACGGAGTGGGCTACCAACCCTATCGGGTCGTAGTTTTG
AGTTTTGAGCTGCTCCACGCCCCTGCGACGGTTTGTGGACCTAA
GAAGAGCACTAACCTCGTGAAAAATAAGTGTGTCAATTTTAATT
TTAATGGCCTGACAGGGACAGGGGTCCTTACAGAGTCCAACAA
AAAGTTTCTTCCTTTTCAGCAGTTTGGGAGGGACATAGCCGATA
CAACCGACGCCGTCCGAGATCCACAAACCTTGGAAATATTGGA
TATCACCCCGTGTAGTTTCGGCGGAGTCAGCGTTATTACCCCTG
GGACCAATACCAGTAACCAGGTAGCCGTATTGTATCAAGGAGT
TAATTGTACTGAAGTGCCTGTGGCTATCCACGCAGACCAACTGA
CCCCGACATGGCGAGTTTACAGTACGGGTTCTAACGTGTTTCAA
ACCAGGGCTGGATGTTTGATCGGTGCAGAACACGTTAATAACA
GTTACGAGTGCGATATCCCGATCGGTGCGGGCATTTGTGCCAGT
TACCAAACACAGACAAACAGTAGAGGTAGTGCTGGTAGCGTTG
CGAGTCAGTCTATTATAGCTTACACGATGTCACTGGGTGCTGAA
AACAGCGTAGCCTATTCTAATAATTCCATCGCCATACCTACAAA
CTTCACCATATCTGTGACGACTGAAATCCTGCCAGTAAGCATGA
CTAAGACATCAGTGGACTGTACTATGTATATCTGTGGCGACTCC
ACAGAGTGTAGTAATCTGTTGCTGCAATACGGGTCTTTCTGTAC
CCAGTTGAATCGCGCATTGACGGGCATAGCCGTTGAACAGGAC
AAGAACACGCAAGAAGTATTCGCACAAGTTAAACAAATCTATA
AGACTCCGCCTATAAAAGACTTTGGTGGATTCAATTTTAGTCAA
ATACTTCCTGATCCGTCCAAGCCGAGTAAACGAAGTTTCATAGA
GGATTTGTTGTTCAATAAGGTGACATTGGCTGACGCAGGCTTTA
TCAAACAGTACGGTGACTGTCTCGGAGACATTGCTGCGCGAGA
CCTTATATGTGCGCAAAAGTTTAACGGACTTACTGTTTTGCCGC
CCTTGTTGACGGACGAAATGATAGCACAATATACTTCCGCTCTG
CTTGCTGGAACAATTACTTCTGGCTGGACTTTCGGAGCGGGTGC
TGCCCTCCAGATACCCTTTGCGATGCAAATGGCTTACAGATTCA
ACGGCATCGGAGTGACTCAGAACGTCCTGTACGAAAACCAAAA
GCTTATCGCAAATCAGTTTAACTCTGCAATCGGAAAAATCCAAG
ACAGCTTGTCTAGTACGGCTAGCGCGCTTGGAAAACTTCAGAAT
GTTGTTAACCAGAACGCGCAAGCACTCAATACGTTGGTTAAGC
AGTTGTCATCTAATTTCGGCGCAATCTCTTCAGTACTTAACGAT
ATTTTGTCAAGGCTCGACCCGCCGGAAGCCGAAGTGCAGATCG
ATAGACTCATAACTGGGAGATTGCAGTCCCTTCAAACCTATGTT
ACACAGCAGTTGATACGAGCGGCCGAGATCAGGGCGTCTGCGA
ACCTTGCCGCAACAAAGATGTCTGAGTGCGTTTTGGGGCAGTCC
AAACGCGTGGATTTCTGCGGTAAAGGGTATCACCTTATGAGTTT
TCCTCAATCAGCGCCCCACGGGGTGGTTTTCCTCCATGTAACGT
ATGTACCCGCACAAGAAAAGAACTTCACCACAGCTCCCGCAAT
ATGCCACGACGGGAAGGCGCACTTCCCGAGGGAAGGTGTTTTC
GTTAGTAACGGCACGCATTGGTTTGTGACCCAGAGAAATTTTTA
CGAGCCACAGATCATCACAACGGACAACACATTCGTAAGTGGA
AACTGTGATGTAGTAATTGGCATCGTCAACAATACCGTCTACGA
CCCTCTCCAACCAGAACTCGATTCTTTTAAGGAGGAACTTGATA
AATATTTTAAAAATCATACAAGCCCCGATGTGGACCTCGGAGA
CATCAGCGGCATTAATGCGAGCGTGGTCAACATCCAAAAGGAG
ATAGATCGCCTCAACGAAGTTGCCAAGAATTTGAATGAGAGCC
TTATAGATCTTCAGGAACTCGGCAAGTACGAGCAATACATTAA
ATGGCCTTGGTACATATGGCTGGGATTTATTGCAGGGCTGATAG
CCATCGTGATGGTGACAATAATGCTCTGTTGTATGACTAGTTGT
TGCAGCTGCCTTAAAGGGTGCTGTTCTTGTGGGAGTTGCTGTAA
GTTTGGCGGAGGCGGAAGCGGAGGCGGAGGCTCCGGCGGAGG
CGGAAGCCCGGTCAAAGGAGGCACCAAGTGCATCAAATACCTG
CTGTTCGGATTTAACTTCATCTTCTGGCTTGCCGGGATTGCTGTC
CTTGCCATTGGACTATGGCTCCGATTCGACTCTCAGACCAAGAG
CATCTTCGAGCAAGAAACTAATAATAATAATTCCAGCTTCTACA
CAGGAGTCTATATTCTGATCGGAGCCGGCGCCCTCATGATGCTG
GTGGGCTTCCTGGGCTGCTGCGGGGCTGTGCAGGAGTCCCAGT
GCATGCTGGGACTGTTCTTCGGCTTCCTCTTGGTGATATTCGCC
ATTGAAATAGCTGCGGCCATCTGGGGATATTCCCACAAGGATG
AGGTGATTAAGGAAGTCCAGGAGTTTTACAAGGACACCTACAA
CAAGCTGAAAACCAAGGATGAGCCCCAGCGGGAAACGCTGAA
AGCCATCCACTATGCGTTGAACTGCTGTGGTTTGGCTGGGGGCG
TGGAACAGTTTATCTCAGACATCTGCCCCAAGAAGGACGTACTC
GAAACCTTCACCGTGAAGTCCTGTCCTGATGCCATCAAAGAGGT
CTTCGACAATAAATTCCACATCATCGGCGCAGTGGGCATCGGC
ATTGCCGTGGTCATGATATTTGGCATGATCTTCAGTATGATCTT
GTGCTGTGCTATCCGCAGGAACCGCGAGATGGTC
SEQ ID NO: 5 provides an amino acid sequence of a coronavirus nucleocapsid protein
polypeptide.
SDNGPQNQRNAPRITFGGPSDSTGSNQNGERSGARSKQRRPQGLP
NNTASWFTALTQHGKEDLKFPRGQGVPINTNSSPDDQIGYYRRAT
RRIRGGDGKMKDLSPRWYFYYLGTGPEAGLPYGANKDGIIWVAT
EGALNTPKDHIGTRNPANNAAIVLQLPQGTTLPKGFYAEGSRGGS
QASSRSSSRSRNSSRNSTPGSSRGTSPARMAGNGGDAALALLLLDR
LNQLESKMSGKGQQQQGQTVTKKSAAEASKKPRQKRTATKAYN
VTQAFGRRGPEQTQGNFGDQELIRQGTDYKHWPQIAQFAPSASAF
FGMSRIGMEVTPSGTWLTYTGAIKLDDKDPNFKDQVILLNKHIDA
YKTFPPTEPKKDKKKKADETQALPQRQKKQQTVTLLPAADLDDFS
KQLQQSMSSADSTQA
SEQ ID NO: 6 provides an amino acid sequence of a nucleocapsid polypeptide-linker-
transmembrane domain proximal region chimera.
WLTYTGAIKLDDKDPNFKDQVILLNKHIDAYKTFPPTEPKKDKKK
KADETQALPQRQKKQQTVTLLPAADLDDFSKQLQQSMSSADSTQ
AKPTTTPAPRPPTPAPTIASQPLSLRPEACRPAAGGAVHTRGLDFAC
DIYIWAPLAGTCGVLLLSLVITLYCNHRNGGGGSGGGGSGGGGSP
VKGGTKCIKYLLFGFNFIFWLAGIAVLAIGLWLRFDSQTKSIFEQET
NNNNSSFYTGVY
SEQ ID NO: 7 provides an amino acid sequence of a nucleocapsid-CD9 fusion protein.
MLLLVTSLLLCELPHPAFLLIPSDNGPQNQRNAPRITFGGPSDSTGS
NQNGERSGARSKQRRPQGLPNNTASWFTALTQHGKEDLKFPRGQ
GVPINTNSSPDDQIGYYRRATRRIRGGDGKMKDLSPRWYFYYLGT
GPEAGLPYGANKDGIIWVATEGALNTPKDHIGTRNPANNAAIVLQ
LPQGTTLPKGFYAEGSRGGSQASSRSSSRSRNSSRNSTPGSSRGTSP
ARMAGNGGDAALALLLLDRLNQLESKMSGKGQQQQGQTVTKKS
AAEASKKPRQKRTATKAYNVTQAFGRRGPEQTQGNFGDQELIRQ
GTDYKHWPQIAQFAPSASAFFGMSRIGMEVTPSGTWLTYTGAIKL
DDKDPNFKDQVILLNKHIDAYKTFPPTEPKKDKKKKADETQALPQ
RQKKQQTVTLLPAADLDDFSKQLQQSMSSADSTQAKPTTTPAPRP
PTPAPTIASQPLSLRPEACRPAAGGAVHTRGLDFACDIYIWAPLAG
TCGVLLLSLVITLYCNHRNGGGGSGGGGSGGGGSPVKGGTKCIKY
LLFGFNFIFWLAGIAVLAIGLWLRFDSQTKSIFEQETNNNNSSFYTG
VYILIGAGALMMLVGFLGCCGAVQESQCMLGLFFGFLLVIFAIEIA
AAIWGYSHKDEVIKEVQEFYKDTYNKLKTKDEPQRETLKAIHYAL
NCCGLAGGVEQFISDICPKKDVLETFTVKSCPDAIKEVFDNKFHIIG
AVGIGIAVVMIFGMIFSMILCCAIRRNREMV
SEQ ID NO: 8 provides a nucleic acid sequence encoding a nucleocapsid-CD9 fusion
protein.
TCTAGAGCCACCATGCTGCTGCTGGTGACCAGCCTGCTGCTGTG
TGAGCTGCCCCACCCCGCCTTTCTGCTGATCCCCTCTGATAATG
GACCCCAAAATCAGCGAAATGCACCCCGCATTACGTTTGGTGG
ACCCTCAGATTCAACTGGCAGTAACCAGAATGGAGAACGCAGT
GGGGCGCGATCAAAACAACGTCGGCCCCAAGGTTTACCCAATA
ATACTGCGTCTTGGTTCACCGCTCTCACTCAACATGGCAAGGAA
GACCTTAAATTCCCTCGAGGACAAGGCGTTCCAATTAACACCA
ATAGCAGTCCAGATGACCAAATTGGCTACTACCGAAGAGCTAC
CAGACGAATTCGTGGTGGTGACGGTAAAATGAAAGATCTCAGT
CCAAGATGGTATTTCTACTACCTAGGAACTGGGCCAGAAGCTG
GACTTCCCTATGGTGCTAACAAAGACGGCATCATATGGGTTGCA
ACTGAGGGAGCCTTGAATACACCAAAAGATCACATTGGCACCC
GCAATCCTGCTAACAATGCTGCAATCGTGCTACAACTTCCTCAA
GGAACAACATTGCCAAAAGGCTTCTACGCAGAAGGGAGCAGAG
GCGGCAGTCAAGCCTCTTCTCGTTCCTCATCACGTAGTCGCAAC
AGTTCAAGAAATTCAACTCCAGGCAGCAGTAGGGGAACTTCTC
CTGCTAGAATGGCTGGCAATGGCGGTGATGCTGCTCTTGCTTTG
CTGCTGCTTGACAGATTGAACCAGCTTGAGAGCAAAATGTCTG
GTAAAGGCCAACAACAACAAGGCCAAACTGTCACTAAGAAATC
TGCTGCTGAGGCTTCTAAGAAGCCTCGGCAAAAACGTACTGCC
ACTAAAGCATACAATGTAACACAAGCTTTCGGCAGACGTGGTC
CAGAACAAACCCAAGGAAATTTTGGGGACCAGGAACTAATCAG
ACAAGGAACTGATTACAAACATTGGCCGCAAATTGCACAATTT
GCCCCCAGCGCTTCAGCGTTCTTCGGAATGTCGCGCATTGGCAT
GGAAGTCACACCTTCGGGAACGTGGTTGACCTACACAGGTGCC
ATCAAATTGGATGACAAAGATCCAAATTTCAAAGATCAAGTCA
TTTTGCTGAATAAGCATATTGACGCATACAAAACATTCCCACCA
ACAGAGCCTAAAAAGGACAAAAAGAAGAAGGCTGATGAAACT
CAAGCCTTACCGCAGAGACAGAAGAAACAGCAAACTGTGACTC
TTCTTCCTGCTGCAGATTTGGATGATTTCTCCAAACAATTGCAA
CAATCCATGAGCAGTGCTGACTCAACTCAGGCCAAGCCCACCA
CCACCCCTGCCCCTAGACCTCCAACCCCAGCCCCTACAATCGCC
AGCCAGCCCCTGAGCCTGAGGCCCGAAGCCTGTAGACCTGCCG
CTGGCGGAGCCGTGCACACCAGAGGCCTGGATTTCGCCTGCGA
CATCTACATCTGGGCCCCTCTGGCCGGCACCTGTGGCGTGCTGC
TGCTGAGCCTGGTCATCACCCTGTACTGCAACCACCGGAATGGC
GGAGGCGGAAGCGGAGGCGGAGGCTCCGGCGGAGGCGGAAGC
CCGGTCAAAGGAGGCACCAAGTGCATCAAATACCTGCTGTTCG
GATTTAACTTCATCTTCTGGCTTGCCGGGATTGCTGTCCTTGCCA
TTGGACTATGGCTCCGATTCGACTCTCAGACCAAGAGCATCTTC
GAGCAAGAAACTAATAATAATAATTCCAGCTTCTACACAGGAG
TCTATATTCTGATCGGAGCCGGCGCCCTCATGATGCTGGTGGGC
TTCCTGGGCTGCTGCGGGGCTGTGCAGGAGTCCCAGTGCATGCT
GGGACTGTTCTTCGGCTTCCTCTTGGTGATATTCGCCATTGAAA
TAGCTGCGGCCATCTGGGGATATTCCCACAAGGATGAGGTGAT
TAAGGAAGTCCAGGAGTTTTACAAGGACACCTACAACAAGCTG
AAAACCAAGGATGAGCCCCAGCGGGAAACGCTGAAAGCCATCC
ACTATGCGTTGAACTGCTGTGGTTTGGCTGGGGGCGTGGAACA
GTTTATCTCAGACATCTGCCCCAAGAAGGACGTACTCGAAACCT
TCACCGTGAAGTCCTGTCCTGATGCCATCAAAGAGGTCTTCGAC
AATAAATTCCACATCATCGGCGCAGTGGGCATCGGCATTGCCGT
GGTCATGATATTTGGCATGATCTTCAGTATGATCTTGTGCTGTG
CTATCCGCAGGAACCGCGAGATGGTCTAA
SEQ ID NO: 9 provides an amino acid sequence of a transmembrane region
polypeptide.
IYIWAPLAGTCGVLLLSLVITLYCNHRN
SEQ ID NO: 10 provides an amino acid sequence of a CD9 protein polypeptide.
PVKGGTKCIKYLLFGFNFIFWLAGIAVLAIGLWLRFDSQTKSIFEQE
TNNNNSSFYTGVYILIGAGALMMLVGFLGCCGAVQESQCMLGLFF
GFLLVIFAIEIAAAIWGYSHKDEVIKEVQEFYKDTYNKLKTKDEPQ
RETLKAIHYALNCCGLAGGVEQFISDICPKKDVLETFTVKSCPDAIK
EVFDNKFHIIGAVGIGIAVVMIFGMIFSMILCCAIRRNREMV
SEQ ID NO: 11 provides a nucleic acid sequence encoding a coronavirus Spike
protein polypeptide.
ATGTTTGTATTTCTGGTACTTTTGCCACTCGTCAGTTCCCAATGT
GTTAACCTGCGCACTCGTACGCAACTCCCCCCAGCATATACCAA
CTCATTTACGCGGGGAGTATATTATCCGGACAAGGTATTTAGGA
GTTCTGTACTGCACAGCACACAGGACCTTTTTCTTCCTTTTTTTT
CCAACGTGACATGGTTTCATGCGATACACGTAAGTGGAACCAA
TGGAACTAAAAGGTTCGATAATCCTGTACTTCCGTTTAACGATG
GTGTCTATTTCGCTTCTACGGAGAAGTCAAATATCATTAGAGGT
TGGATCTTCGGTACAACTCTTGACAGTAAGACGCAAAGTCTGCT
CATCGTAAACAACGCGACGAACGTAGTGATAAAGGTCTGCGAG
TTTCAGTTCTGTAATGATCCGTTTCTTGACGTCTATTACCATAAG
AACAATAAGAGTTGGATGGAAAGTGGAGTATACAGCAGCGCTA
ACAATTGCACATTTGAATATGTCTCTCAACCTTTCCTGATGGAC
CTCGAAGGGAAACAAGGTAACTTCAAAAATCTCCGAGAATTTG
TTTTTAAAAATATAGACGGATATTTCAAGATATACTCAAAGCAC
ACGCCGATAAATCTGGTCAGGGATCTCCCTCAGGGTTTCAGTGC
ACTCGAACCTTTGGTAGATTTGCCTATCGGAATAAATATTACAC
GCTTTCAGACGCTTCTTGCGTTGCACCGGTCTTACCTTACACCTG
GGGACAGTTCATCTGGTTGGACGGCCGGGGCTGCCGCTTATTAC
GTCGGATATCTCCAACCAAGAACGTTTCTGCTTAAATATAATGA
GAACGGCACAATAACTGACGCGGTGGATTGCGCTCTTGATCCTC
TCTCAGAGACCAAATGCACGCTCAAATCCTTTACTGTCGAGAAA
GGGATTTATCAGACCAGCAACTTTCGCGTGCAGCCTACAGAAA
GTATTGTAAGATTCCCTAACATTACTAACCTCTGCCCGTTCGGC
GAGGTATTTAATGCAACACGATTTGCAAGTGTCTATGCGTGGAA
TAGAAAACGCATTTCAAATTGTGTCGCCGATTACTCCGTACTCT
ATAACAGCGCCTCATTTAGCACGTTTAAATGTTATGGTGTCTCC
CCCACAAAGCTCAATGATCTCTGCTTCACTAACGTGTATGCTGA
TAGCTTCGTGATCCGAGGTGACGAAGTGAGGCAAATTGCTCCG
GGTCAAACCGGGAAGATCGCAGATTATAACTATAAGTTGCCAG
ACGATTTCACTGGATGTGTTATTGCGTGGAACTCTAATAACCTG
GACTCAAAAGTGGGAGGGAATTACAATTACCGCTACAGGCTGT
TTCGCAAAAGCAATTTGAAACCCTTCGAGAGGGACATCAGTAC
TGAGATATACCAGGCAGGGTCAAAACCCTGCAATGGAGTCGAA
GGATTTAATTGCTACTTCCCGCTTCAGAGCTATGGGTTCCAGCC
AACGAACGGAGTGGGCTACCAACCCTATCGGGTCGTAGTTTTG
AGTTTTGAGCTGCTCCACGCCCCTGCGACGGTTTGTGGACCTAA
GAAGAGCACTAACCTCGTGAAAAATAAGTGTGTCAATTTTAATT
TTAATGGCCTGACAGGGACAGGGGTCCTTACAGAGTCCAACAA
AAAGTTTCTTCCTTTTCAGCAGTTTGGGAGGGACATAGCCGATA
CAACCGACGCCGTCCGAGATCCACAAACCTTGGAAATATTGGA
TATCACCCCGTGTAGTTTCGGCGGAGTCAGCGTTATTACCCCTG
GGACCAATACCAGTAACCAGGTAGCCGTATTGTATCAAGGAGT
TAATTGTACTGAAGTGCCTGTGGCTATCCACGCAGACCAACTGA
CCCCGACATGGCGAGTTTACAGTACGGGTTCTAACGTGTTTCAA
ACCAGGGCTGGATGTTTGATCGGTGCAGAACACGTTAATAACA
GTTACGAGTGCGATATCCCGATCGGTGCGGGCATTTGTGCCAGT
TACCAAACACAGACAAACAGTAGAGGTAGTGCTGGTAGCGTTG
CGAGTCAGTCTATTATAGCTTACACGATGTCACTGGGTGCTGAA
AACAGCGTAGCCTATTCTAATAATTCCATCGCCATACCTACAAA
CTTCACCATATCTGTGACGACTGAAATCCTGCCAGTAAGCATGA
CTAAGACATCAGTGGACTGTACTATGTATATCTGTGGCGACTCC
ACAGAGTGTAGTAATCTGTTGCTGCAATACGGGTCTTTCTGTAC
CCAGTTGAATCGCGCATTGACGGGCATAGCCGTTGAACAGGAC
AAGAACACGCAAGAAGTATTCGCACAAGTTAAACAAATCTATA
AGACTCCGCCTATAAAAGACTTTGGTGGATTCAATTTTAGTCAA
ATACTTCCTGATCCGTCCAAGCCGAGTAAACGAAGTTTCATAGA
GGATTTGTTGTTCAATAAGGTGACATTGGCTGACGCAGGCTTTA
TCAAACAGTACGGTGACTGTCTCGGAGACATTGCTGCGCGAGA
CCTTATATGTGCGCAAAAGTTTAACGGACTTACTGTTTTGCCGC
CCTTGTTGACGGACGAAATGATAGCACAATATACTTCCGCTCTG
CTTGCTGGAACAATTACTTCTGGCTGGACTTTCGGAGCGGGTGC
TGCCCTCCAGATACCCTTTGCGATGCAAATGGCTTACAGATTCA
ACGGCATCGGAGTGACTCAGAACGTCCTGTACGAAAACCAAAA
GCTTATCGCAAATCAGTTTAACTCTGCAATCGGAAAAATCCAAG
ACAGCTTGTCTAGTACGGCTAGCGCGCTTGGAAAACTTCAGAAT
GTTGTTAACCAGAACGCGCAAGCACTCAATACGTTGGTTAAGC
AGTTGTCATCTAATTTCGGCGCAATCTCTTCAGTACTTAACGAT
ATTTTGTCAAGGCTCGACCCGCCGGAAGCCGAAGTGCAGATCG
ATAGACTCATAACTGGGAGATTGCAGTCCCTTCAAACCTATGTT
ACACAGCAGTTGATACGAGCGGCCGAGATCAGGGCGTCTGCGA
ACCTTGCCGCAACAAAGATGTCTGAGTGCGTTTTGGGGCAGTCC
AAACGCGTGGATTTCTGCGGTAAAGGGTATCACCTTATGAGTTT
TCCTCAATCAGCGCCCCACGGGGTGGTTTTCCTCCATGTAACGT
ATGTACCCGCACAAGAAAAGAACTTCACCACAGCTCCCGCAAT
ATGCCACGACGGGAAGGCGCACTTCCCGAGGGAAGGTGTTTTC
GTTAGTAACGGCACGCATTGGTTTGTGACCCAGAGAAATTTTTA
CGAGCCACAGATCATCACAACGGACAACACATTCGTAAGTGGA
AACTGTGATGTAGTAATTGGCATCGTCAACAATACCGTCTACGA
CCCTCTCCAACCAGAACTCGATTCTTTTAAGGAGGAACTTGATA
AATATTTTAAAAATCATACAAGCCCCGATGTGGACCTCGGAGA
CATCAGCGGCATTAATGCGAGCGTGGTCAACATCCAAAAGGAG
ATAGATCGCCTCAACGAAGTTGCCAAGAATTTGAATGAGAGCC
TTATAGATCTTCAGGAACTCGGCAAGTACGAGCAATACATTAA
ATGGCCTTGGTACATATGGCTGGGATTTATTGCAGGGCTGATAG
CCATCGTGATGGTGACAATAATGCTCTGTTGTATGACTAGTTGT
TGCAGCTGCCTTAAAGGGTGCTGTTCTTGTGGGAGTTGCTGTAA
GTTT
SEQ ID NO: 12 provides a nucleic acid sequence encoding a coronavirus nucleocapsid
protein polypeptide.
TCTGATAATGGACCCCAAAATCAGCGAAATGCACCCCGCATTA
CGTTTGGTGGACCCTCAGATTCAACTGGCAGTAACCAGAATGG
AGAACGCAGTGGGGCGCGATCAAAACAACGTCGGCCCCAAGGT
TTACCCAATAATACTGCGTCTTGGTTCACCGCTCTCACTCAACA
TGGCAAGGAAGACCTTAAATTCCCTCGAGGACAAGGCGTTCCA
ATTAACACCAATAGCAGTCCAGATGACCAAATTGGCTACTACC
GAAGAGCTACCAGACGAATTCGTGGTGGTGACGGTAAAATGAA
AGATCTCAGTCCAAGATGGTATTTCTACTACCTAGGAACTGGGC
CAGAAGCTGGACTTCCCTATGGTGCTAACAAAGACGGCATCAT
ATGGGTTGCAACTGAGGGAGCCTTGAATACACCAAAAGATCAC
ATTGGCACCCGCAATCCTGCTAACAATGCTGCAATCGTGCTACA
ACTTCCTCAAGGAACAACATTGCCAAAAGGCTTCTACGCAGAA
GGGAGCAGAGGCGGCAGTCAAGCCTCTTCTCGTTCCTCATCACG
TAGTCGCAACAGTTCAAGAAATTCAACTCCAGGCAGCAGTAGG
GGAACTTCTCCTGCTAGAATGGCTGGCAATGGCGGTGATGCTGC
TCTTGCTTTGCTGCTGCTTGACAGATTGAACCAGCTTGAGAGCA
AAATGTCTGGTAAAGGCCAACAACAACAAGGCCAAACTGTCAC
TAAGAAATCTGCTGCTGAGGCTTCTAAGAAGCCTCGGCAAAAA
CGTACTGCCACTAAAGCATACAATGTAACACAAGCTTTCGGCA
GACGTGGTCCAGAACAAACCCAAGGAAATTTTGGGGACCAGGA
ACTAATCAGACAAGGAACTGATTACAAACATTGGCCGCAAATT
GCACAATTTGCCCCCAGCGCTTCAGCGTTCTTCGGAATGTCGCG
CATTGGCATGGAAGTCACACCTTCGGGAACGTGGTTGACCTAC
ACAGGTGCCATCAAATTGGATGACAAAGATCCAAATTTCAAAG
ATCAAGTCATTTTGCTGAATAAGCATATTGACGCATACAAAACA
TTCCCACCAACAGAGCCTAAAAAGGACAAAAAGAAGAAGGCT
GATGAAACTCAAGCCTTACCGCAGAGACAGAAGAAACAGCAA
ACTGTGACTCTTCTTCCTGCTGCAGATTTGGATGATTTCTCCAAA
CAATTGCAACAATCCATGAGCAGTGCTGACTCAACTCAGGCC
SEQ ID NO: 13 provides a nucleic acid sequence encoding a CD9 exosomal tetraspanin
protein polypeptide.
TCTGATAATGGACCCCAAAATCAGCGAAATGCACCCCGCATTA
CGTTTGGTGGACCCTCAGATTCAACTGGCAGTAACCAGAATGG
AGAACGCAGTGGGGCGCGATCAAAACAACGTCGGCCCCAAGGT
TTACCCAATAATACTGCGTCTTGGTTCACCGCTCTCACTCAACA
TGGCAAGGAAGACCTTAAATTCCCTCGAGGACAAGGCGTTCCA
ATTAACACCAATAGCAGTCCAGATGACCAAATTGGCTACTACC
GAAGAGCTACCAGACGAATTCGTGGTGGTGACGGTAAAATGAA
AGATCTCAGTCCAAGATGGTATTTCTACTACCTAGGAACTGGGC
CAGAAGCTGGACTTCCCTATGGTGCTAACAAAGACGGCATCAT
ATGGGTTGCAACTGAGGGAGCCTTGAATACACCAAAAGATCAC
ATTGGCACCCGCAATCCTGCTAACAATGCTGCAATCGTGCTACA
ACTTCCTCAAGGAACAACATTGCCAAAAGGCTTCTACGCAGAA
GGGAGCAGAGGCGGCAGTCAAGCCTCTTCTCGTTCCTCATCACG
TAGTCGCAACAGTTCAAGAAATTCAACTCCAGGCAGCAGTAGG
GGAACTTCTCCTGCTAGAATGGCTGGCAATGGCGGTGATGCTGC
TCTTGCTTTGCTGCTGCTTGACAGATTGAACCAGCTTGAGAGCA
AAATGTCTGGTAAAGGCCAACAACAACAAGGCCAAACTGTCAC
TAAGAAATCTGCTGCTGAGGCTTCTAAGAAGCCTCGGCAAAAA
CGTACTGCCACTAAAGCATACAATGTAACACAAGCTTTCGGCA
GACGTGGTCCAGAACAAACCCAAGGAAATTTTGGGGACCAGGA
ACTAATCAGACAAGGAACTGATTACAAACATTGGCCGCAAATT
GCACAATTTGCCCCCAGCGCTTCAGCGTTCTTCGGAATGTCGCG
CATTGGCATGGAAGTCACACCTTCGGGAACGTGGTTGACCTAC
ACAGGTGCCATCAAATTGGATGACAAAGATCCAAATTTCAAAG
ATCAAGTCATTTTGCTGAATAAGCATATTGACGCATACAAAACA
TTCCCACCAACAGAGCCTAAAAAGGACAAAAAGAAGAAGGCT
GATGAAACTCAAGCCTTACCGCAGAGACAGAAGAAACAGCAA
ACTGTGACTCTTCTTCCTGCTGCAGATTTGGATGATTTCTCCAAA
CAATTGCAACAATCCATGAGCAGTGCTGACTCAACTCAGGCC

### Extracellular Vesicles and Exosomes

A variety of host cells are known in the art and suitable for proteins expression and extracellular vesicles production. Non-limiting examples of typical cell used for transfection include, but are not limited to, a bacterial cell, a eukaryotic cell, a yeast cell, an insect cell, or a plant cell. For example, human embryonic kidney 293 (HEK293), E. coli, Bacillus, Streptomyces, Pichia pastoris, Salmonella typhimurium, Drosophila S2, Spodoptera SJ9, CHO, COS (e.g. COS-7), 3T3-F442A, HeLa, HUVEC, HUAEC, NIH 3T3, Jurkat, 293, 293H, or 293F. See, e.g., Portolano et al., “Recombinant Protein Expression for Structural Biology in HEK 293F Suspension Cells: A Novel and Accessible Approach,” Journal of Visualized Experiments, October 2014, 92, e51897, pp. 1-8 for a description of the recombinant proteins in 293 cells in suspension culture.

Extracellular vesicles (EVs) are lipid bound vesicles secreted by cells into the extracellular space. The three main subtypes of EVs are microvesicles (MVs), exosomes, and apoptotic bodies, which are differentiated based upon their biogenesis, release pathways, size, content, and function. For a review of extracellular vesicles, see, e.g., Doyle and Wang, “Overview of Extracellular Vesicles, Their Origin, Composition, Purpose, and Methods for Exosome Isolation and Analysis,” Cells, v. 8(7), 2019 July, and references therein.

Exosomes include small, secreted vesicles of about 20-200 nm in diameter that are released by inter alia mammalian cells, and made either by budding into endosomes or by budding from the plasma membrane of a cell. In some cases, exosomes have a characteristic buoyant density of approximately 1.1-1.2 g/mL, and a characteristic lipid composition. Their lipid membrane is typically rich in cholesterol and contains sphingomyelin, ceramide, lipid rafts and exposed phosphatidylserine. Exosomes express certain marker proteins, such as integrins and cell adhesion molecules, but generally lack markers of lysosomes, mitochondria, or caveolae. In some embodiments, the exosomes contain cell-derived components, such as, but not limited to, proteins, DNA and RNA (e.g., microRNA [miR] and noncoding RNA). In some embodiments, exosomes can be obtained from cells obtained from a source that is allogeneic, autologous, xenogeneic, or syngeneic with respect to the recipient of the exosomes.

Certain types of RNA, e.g., microRNA (miRNA), are known to be carried by exosomes. miRNAs function as post-transcriptional regulators, often through binding to complementary sequences on target messenger RNA transcripts (mRNAs), thereby resulting in translational repression, target mRNA degradation and/or gene silencing.

Useful exosomes can be obtained from any cell source, including prokaryotes, plants, fungi, metazoans, vertebrate, mammalian, primate, human, autologous cells and allogeneic cells. See, e.g., Kim et al., “Platform technologies and human cell lines for the production of therapeutic exosomes,” Extracell Vesicles Circ Nucleic Acids 2021; 2:3-17. For example, exosomes may be derived from mesenchymal stem cells, embryonic stem cells, iPS cells, immune cells, PBMCs, neural stem cells, HEK293 cells, which are described in e.g., Dumont et al., “Human cell lines for biopharmaceutical manufacturing: history, status, and future perspectives,” Crit Rev Biotechnol 2016; 36:1110-22, HEK293T cells, which are described in e.g., Li et al., “Identification and characterization of 293T cell-derived exosomes by profiling the protein, mRNA and MicroRNA components,” PLoS One 2016; 11:e0163043, 293F cells, Stenkamp et al., “Exosomes represent a novel mechanism of regulatory T cell suppression (P1079),” J Immunol May 1, 2013, 190 (1 Supplement) 121.11, amniotic cells, CAR-T cells, cardiospheres and cardiosphere-derived cells (CDCs), which are described in, e.g., WO2014028493, WO2022006178A1, US20210032598A1, U.S. Pat. No. 9,828,603B2, EP2914273A1, US20200316226A1, US20120315252A1, US20170360842A1, and references therein, and the like.

Briefly, methods for preparing exosomes can include the steps of culturing cells in media, isolating the cells from the media, purifying the exosome by, e.g., sequential centrifugation, and optionally, clarifying the exosomes on a density gradient, e.g., sucrose density gradient. In some instances, the isolated and purified exosomes are essentially free of non-exosome components, such as cellular components or whole cells. Exosomes can be resuspended in a buffer such as a sterile PBS buffer containing 0.01-1% human serum albumin. The exosomes may be frozen and stored for future use.

Exosomes can be collected, concentrated and/or purified using methods known in the art. For example, differential centrifugation has become a leading technique wherein secreted exosomes are isolated from the supernatants of cultured cells. This approach allows for separation of exosomes from larger extracellular vesicles and from most non-particulate contaminants by exploiting their size. Exosomes can be prepared as described in a wide array of papers, including but not limited to, Fordjour et al., “A shared pathway of exosome biogenesis operates at plasma and endosome membranes”, bioRxiv, preprint posted Feb. 11, 2019, at https://www.biorxiv.org/content/10.1101/545228v1; Booth et ah, “Exosomes and HIV Gag bud from endosome-like domains of the T cell plasma membrane”, J Cell Biol., 172:923-935 (2006); and, Fang et ah, “Higher-order oligomerization targets plasma membrane proteins and HIV gag to exosomes”, PLoS Biol., 5:e158 (2007). Exosomes using a commercial kit such as, but not limited to the ExoSpin™ Exosome Purification Kit, Invitrogen® Total Exosome Purification Kit, PureExo® Exosome Isolation Kit, and ExoCap™ Exosome Isolation kit. Methods for isolating exosome from stem cells are found in, e.g., Tan et ah, Journal of Extracellular Vesicles, 2:22614 (2013); Ono et ah, Sci Signal, 7(332):ra63 (2014) and U.S. Application Publication Nos. 2012/0093885 and 2014/0004601. Methods for isolating exosome from cardiosphere-derived cells are found in, e.g., Ibrahim et al., “Exosomes as critical agents of cardiac regeneration triggered by cell therapy,” Stem Cell Reports, 2014. Specific methodologies include ultracentrifugation, density gradient, HPLC, adherence to substrate based on affinity, or filtration based on size exclusion.

Size exclusion allows for their separation from biochemically similar, but biophysically different microvesicles, which possess larger diameters of up to 1,000 nm. Differences in flotation velocity further allows for separation of differentially sized exosomes. In general, exosome sizes will possess a diameter ranging from 30-200 nm, including sizes of 40-100 nm. Further purification may rely on specific properties of the particular exosomes of interest. This includes, e.g., use of immunoadsorption with a protein of interest to select specific vesicles with exoplasmic or outward orientations.

Among current methods, e.g., differential centrifugation, discontinuous density gradients, immunoaffinity, ultrafiltration and high-performance liquid chromatography (HPLC), differential ultracentrifugation is the most commonly used for exosome isolation. This technique utilizes increasing centrifugal force from 2,000×g to 10,000×g to separate the medium- and larger-sized particles and cell debris from the exosome pellet at 100,000×g. Centrifugation alone allows for significant separation/collection of exosomes from a conditioned medium, although it may be insufficient to remove various protein aggregates, genetic materials, particulates from media and cell debris that are common contaminants. Enhanced specificity of exosome purification may deploy sequential centrifugation in combination with ultrafiltration, or equilibrium density gradient centrifugation in a sucrose density gradient, to provide for the greater purity of the exosome preparation (flotation density 1.1-1.2 g/mL) or application of a discrete sugar cushion in preparation.

Ultrafiltration can be used to purify exosomes without compromising their biological activity. Membranes with different pore sizes—such as 100 kDa molecular weight cutoff (MWCO) and gel filtration to eliminate smaller particles—have been used to avoid the use of a nonneutral pH or non-physiological salt concentration. Currently available tangential flow filtration (TFF) systems are scalable (to >10,000 L), allowing one to not only purify, but concentrate the exosome fractions, and such approaches are less time consuming than differential centrifugation. HPLC can also be used to purify exosomes to more uniformly sized particle preparations and preserve their biological activity as the preparation is maintained at a physiological pH and salt concentration. Other chemical methods have exploit differential solubility of exosomes for precipitation techniques, addition to volume-excluding polymers (e.g., polyethylene glycols (PEGs)), possibly combined additional rounds of centrifugation or filtration. For example, a precipitation reagent, ExoQuick®, can be added to conditioned cell media to quickly and rapidly precipitate a population of exosomes, although re-suspension of pellets prepared via this technique may be difficult. Flow field-flow fractionation (FlFFF) is an elution-based technique that is used to separate and characterize macromolecules (e.g., proteins) and nano- to micro-sized particles (e.g., organelles and cells) and which has been successfully applied to fractionate exosomes from culture media.

Beyond these techniques relying on general biochemical and biophysical features, focused techniques may be applied to isolate specific exosomes of interest. This includes relying on antibody immunoaffinity to recognizing certain exosome-associated antigens. As described, exosomes further express the extracellular domain of membrane-bound receptors at the surface of the membrane. This presents an opportunity for isolating and segregating exosomes in connection with their parental cellular origin, based on a shared antigenic profile. Conjugation to magnetic beads (e.g., such as anti-CD81 magnetic beads), chromatography matrices, plates or microfluidic devices allows isolating of specific exosome populations of interest as may be related to their production from a parent cell of interest or associated cellular regulatory state. Other affinity-capture methods use lectins which bind to specific saccharide residues on the exosome surface.

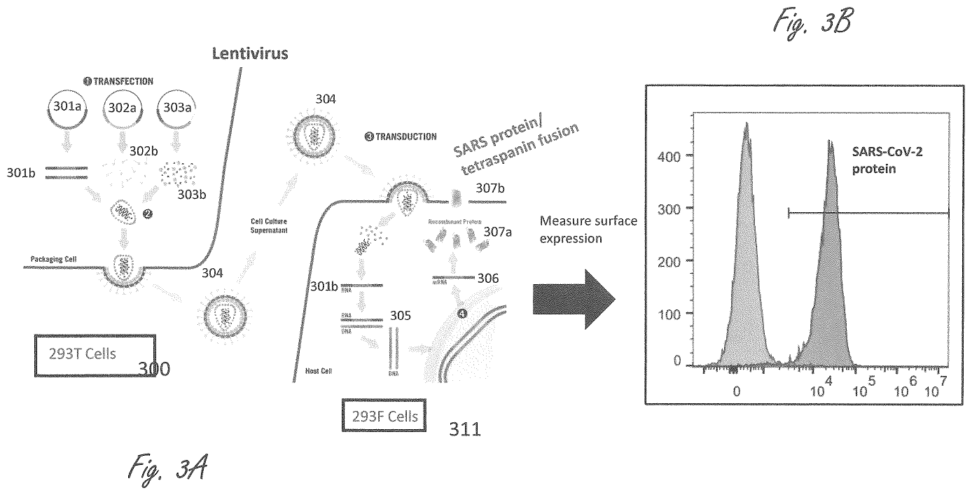

For example, exosomes (and other extracellular vesicles) may be produced via 293F cells. The 293F cells may be transfected with (or transduced with a lentivirus bearing) a polynucleotide that encodes a spike protein or a nucleocapsid protein, or chimeral fusions thereof, as described herein (see FIG. 3A), and expressing the spike protein or nucleocapsid protein, such that the spike protein or nucleocapsid is sorted into and displayed in or on the exosomes isolated therefrom. An example procedure for making exosomes from 293F cells may include steps as follows: 293F cells (Gibco™, Cat. #51-0029, ThermoFisher Scientific, Waltham, MA) may be tested for pathogens and found to be free of viral (cytomegalovirus, human immunodeficiency virus I and II, Epstein Barr virus, hepatitis B virus, and parvovirus B19) and bacterial (Mycoplasma) contaminants. Cells may be maintained in FreeStyle™ 293 Expression Medium (Gibco, Cat. #12338-018, ThermoFisher Scientific, Waltham, MA) and incubated at 37° C. in 8% CO2. For exosome production, 293F cells may be seeded at a density of 1.5E6 cells/ml in shaker flasks in a volume of about ¼ the flask volume and grown at a shaking speed of about 110 rpm. HEK293 cells may be grown in Dulbecco's modified Eagle's medium (DMEM) supplemented with 10% fetal calf serum.

To purify exosomes, the 293F cells may be cultured in shaker flasks for a period of three days. Cells and large cell debris may be removed by centrifugation at 300×g for 5 minutes followed by 3,000×g for 15 minutes. The resulting supernatant may be passed through a 0.22 μm sterile filtration filter unit (Thermo Fisher, Cat. #566-0020) to generate a clarified tissue culture supernatant (CTCS). The CTCS may be concentrated by centrifugal filtration (Centricon Plus-70, Ultracel-PL Membrane, 100 kDa size exclusion, Millipore Sigma, Cat. #UFC710008, St. Louis, MO), with about 120 mL CTCS concentrated to about 0.5 mL. Concentrated CTCS my then be purified by size exclusion chromatography (SEC) in 1× PBS (qEV original columns/35 nm: Izon Science, Cat. #SP5), with the exosomes present in each 0.5 ml starting sample eluting in three 0.5 ml fractions. Purified exosomes may be reconcentrated using Amicon® Ultra-4 100 kDa cutoff spin columns (Cat. #UFC810024). This process may yield a population of exosomes/small EVs that have the expected ultrastructure and size distribution profile of human exosomes and contain the exosomal marker proteins CD9 and CD63, at a concentrating effect of about 500-fold, to a final concentration of 1E10-2E12 exosomes/ml. The concentration and size of the isolated extracellular vesicles may be measured using NANOSIGHT nanoparticle tracking analysis system (Malvern Panalytical, Malvern, UK).

### SARS-CoV-2 Proteins

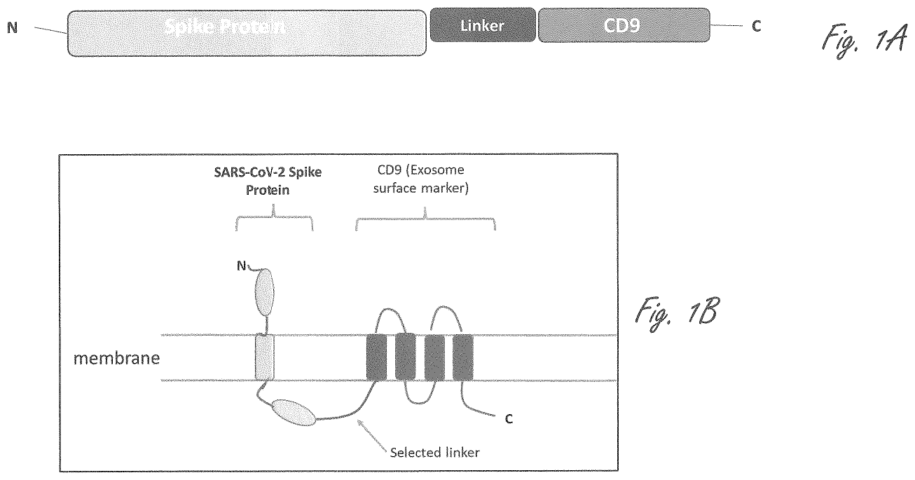

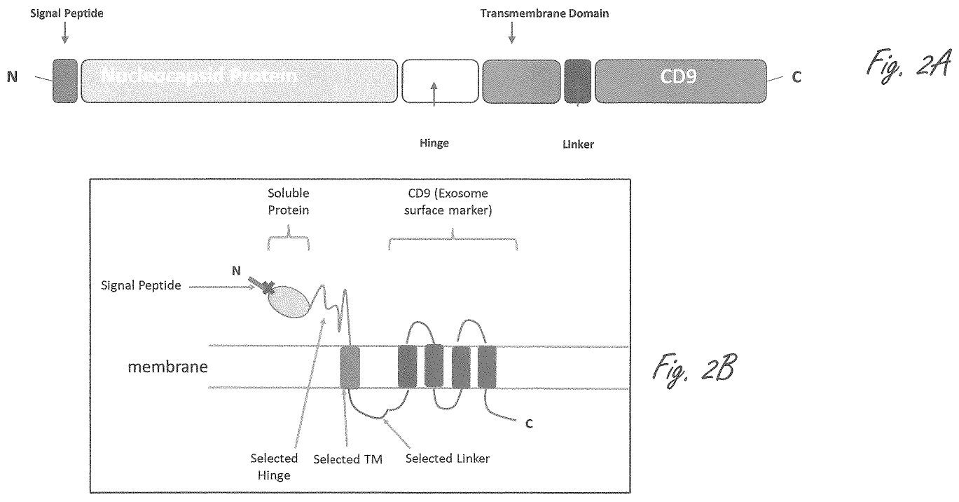

Disclosed are membrane-bound vesicles that contain one or more populations of SARS-CoV-2 structural proteins. By contain, what is meant is that the contained protein may be within the lumen of the vesicle, displayed on the surface of the vesicle, or both within the lumen and on the surface. Here, those fusion proteins containing a tetraspanin protein polypeptide sequence are mostly displayed on the surface of the vesicle. Those proteins that are displayed on the surface of the vesicle may have a portion of the protein inside the lumen, a portion of the protein spanning the membrane of the vesicle, i.e., a transmembrane spanning region or domain, and a portion of the protein extending outside the vesicle. In one embodiment, the SARS-CoV-2 structural protein is a spike glycoprotein (S), a nucleocapsid (N) protein, a membrane (M) protein, or an envelope (E) protein, or any combination thereof. See Satarker and Nampoothiri, “Structural Proteins in Severe Acute Respiratory Syndrome Coronavirus-2,” Arch Med Res. 2020 August; 51(6): 482-491. See, e.g., FIGS. 1A, 1B, 2A, and 2C, which depict spike/CD9 and nucleocapsid/CD fusion proteins for expression of spike or nucleocapsid antigen on the surface of the exosomes.

In one embodiment, the antigenic protein is SARS-CoV-2 spike glycoprotein (a.k.a. spike protein or simply “spike”). The spike protein can be of any variant of SARS-CoV-2, such as, e.g., the Wuhan-1 strain, an omicron variant (e.g., BA.2 variant), a delta variant (e.g., B.1.617.2, AY.3, AY.103, AY.44, AY.43 variant, or the like), and epsilon variant (e.g., B.1.427 or B.1.429 variant), or any variant now known or yet to be discovered. As used herein, the term spike refers to any SARS-CoV-2 spike glycoprotein, chimera, or fragment thereof unless otherwise specified.

In some embodiments, the SARS-CoV-2 spike protein is the Wuhan-1 strain SARS-CoV-2 spike protein or a Delta variant SARS-CoV-2 spike protein; a furin-blocked, trimer-stabilized form of the Wuhan-1 strain SARS-CoV-2 spike protein; the Wuhan-1 strain SARS-CoV-2 spike protein with an amino acid change of D614G; the Wuhan-1 strain SARS-CoV-2 spike protein with di-proline substitutions of 986KV987-to-986PP987 (S-2P); and/or the Wuhan-1 strain SARS-CoV-2 spike protein with cleavage site mutations of 682RRAR685-to-682GSAG685, or equivalent (S-CSM).

### Extracellular Vesicles Displaying Spike Protein or Nucleocapsid Protein

In one embodiment, the invention provides extracellular vesicles that express on their surface (a.k.a. “display”) spike protein or nucleocapsid that are useful as a vaccine against multiple variants of SARS-CoV-2. The spike protein may be a delta variant with any one or more of trimer stabilization mutation, a prefusion conformation stabilization mutation (e.g., di-proline stabilization mutations), and a furin cleavage site mutation. See, e.g., Walls et al., “Structure, Function, and Antigenicity of the SARS-CoV-2 Spike Glycoprotein,” Cell 180, 281-292, Apr. 16, 2020; Wrapp et al., “Cryo-EM structure of the 2019-nCoV spike in the prefusion conformation,” Science 367, 1260-1263 (2020)13 Mar. 2020; Kirchdoerfer et al., “Stabilized coronavirus spikes are resistant to conformational changes induced by receptor recognition or proteolysis,” Sci Rep 8, 15701 (2018), doi.org/10.1038/s41598-018-34171-7; Pallesen et al., “Immunogenicity and structures of a rationally designed prefusion MERS-CoV spike antigen,” PNAS, E7348-E7357, published online Aug. 14, 2017 pnas.org/cgi/doi/10.1073/pnas.1707304114; Juraszek, et al., “Stabilizing the closed SARS-CoV-2 spike trimer,” Nat Commun 12, 244 (2021), doi.org/10.1038/s41467-020-20321-x; Johnson, 2020; and Xiong 2020; and references therein.

Here, synthetic fusion proteins containing a C-terminal tetraspanin protein and either a SARS-CoV-2 spike protein (FIGS. 1A and 1B) or a SARS-CoV-2 nucleocapsid protein (FIGS. 2A and 2B) were produced. It is important to note that nucleocapsid protein is a soluble protein that is not expressed at the virus surface, therefore the engineered fusion protein that includes tetraspanin and other transmembrane domains enables the placement of nucleocapsid protein at the vesicle surface to provide a readily accessible antigen for immunization (see FIG. 2B).

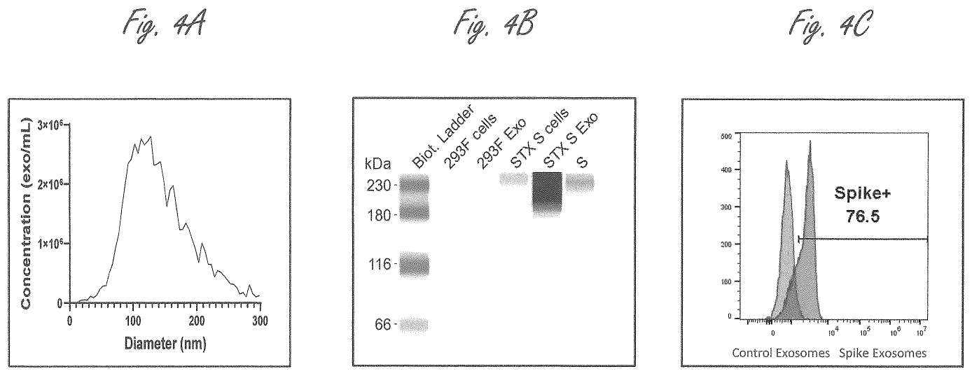

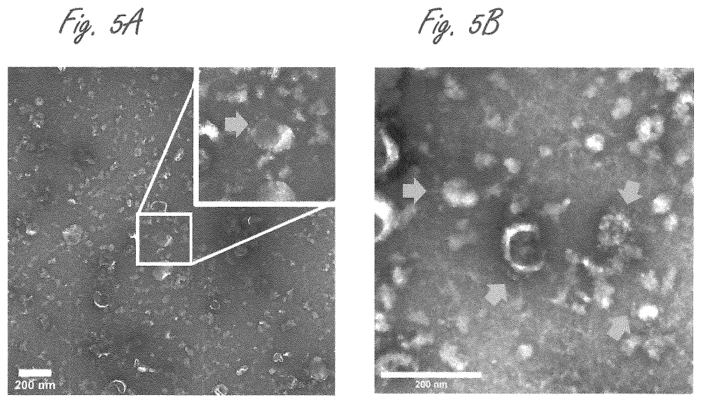

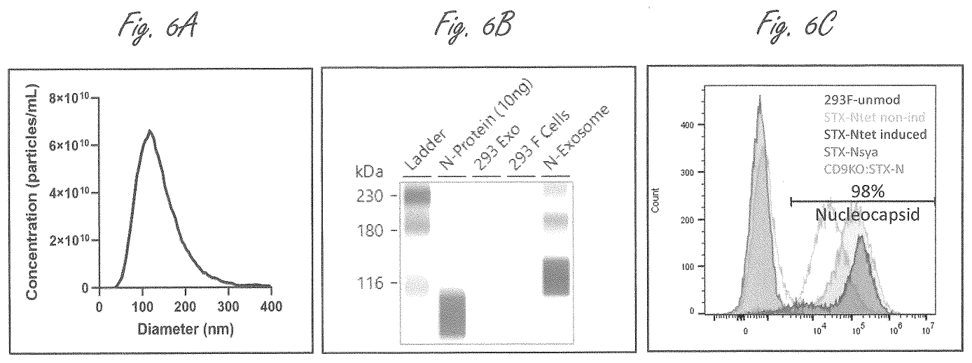

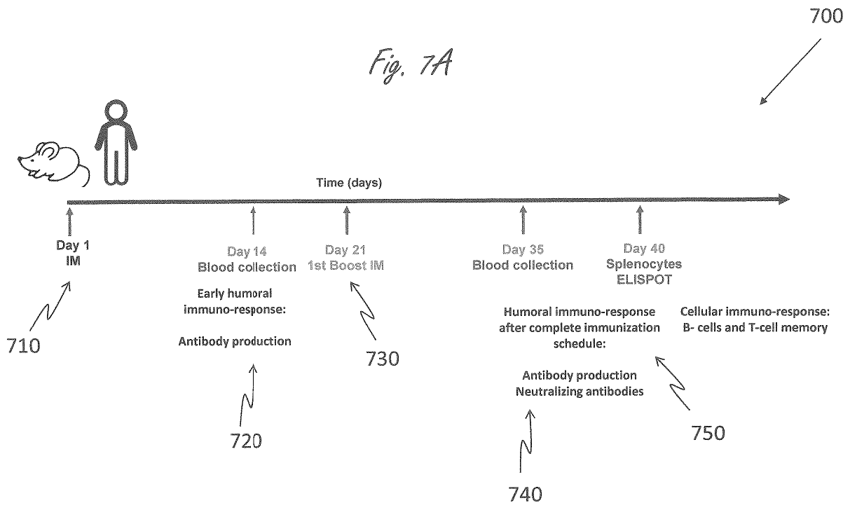

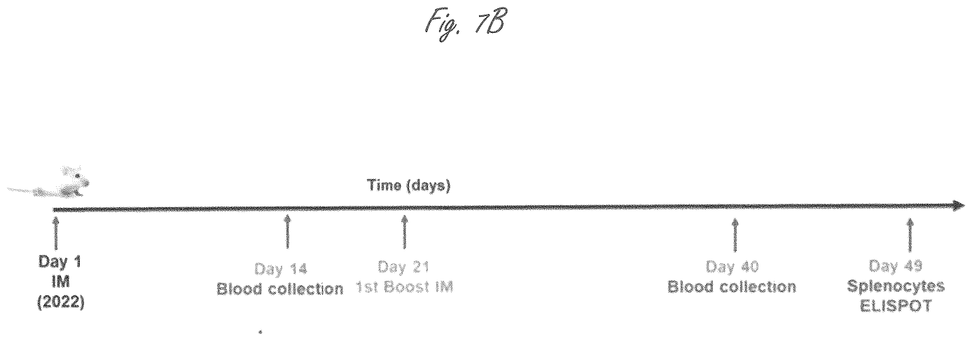

In one embodiment, the exosomes that express spike protein or nucleocapsid protein on their surface were made from spike/nuclceocapsid protein-expressing 293F cells. Turning to FIG. 3A, in specific exemplar embodiment, a packaging cell (300) was transfected with a plasmid that encodes the spike or nucleocapsid-tetraspanin fusion protein (301a) and plasmids that encode lentivirus structural proteins (302a and 303a). The lentivirus proteins were produced (302b and 303b) and incorporated the fusion protein RNA to form a lentivirus vector containing the fusion protein RNA (304). A host cell (311) was transduced with the fusion protein RNA-bearing lentivirus (304), enabling the production of the SARS-CoV-2 spike or nucleocapsid-tetraspanin fusion protein (307a), and its sorting to the plasma membrane (307b) (FIG. 3B) and the surface of exosomes that were produced by the transduced host cells (FIGS. 4C, 5A, 5B, and 6C).

Turning to FIGS. 4A-C and 5A and 5B, exosomes were isolated from 293F cells that harbored the spike-CD9 construct (FIG. 1A and FIG. 1B). FIG. 4A shows the size distribution of those exosomes from about 50 nm to about 270 nm, with a median of about 100-150 nm. FIG. 4B shows expression of spike-containing fusion protein in the transduced 293F cells (lane 4), and enriched expression/display of the spike-containing fusion protein in the exosomes that were derived from the transduced 293F cells (lane 5). FIG. 4C demonstrates significant expression of spike-containing fusion protein on those exosomes as determined by spike flow cytometry.

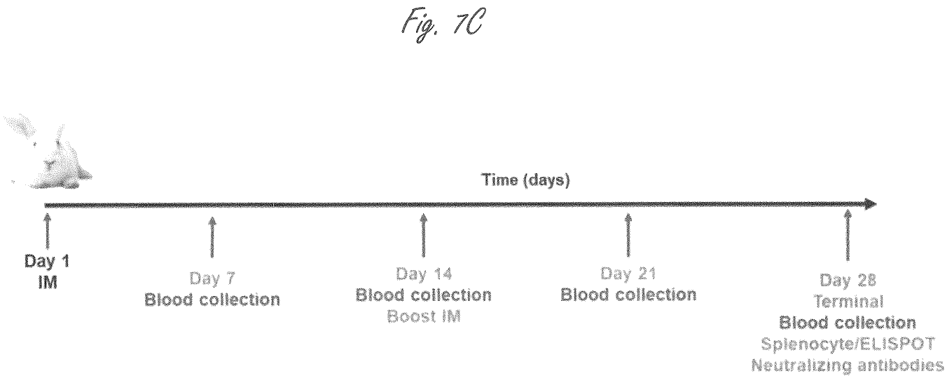

Transmission electron micrography confirmed that spike protein was expressed on the surface of the exosomes derived from the spike-CD9 transduced 293F cells (arrows at FIGS. 5A and 5B point to spikes at the surface of the vesicles).

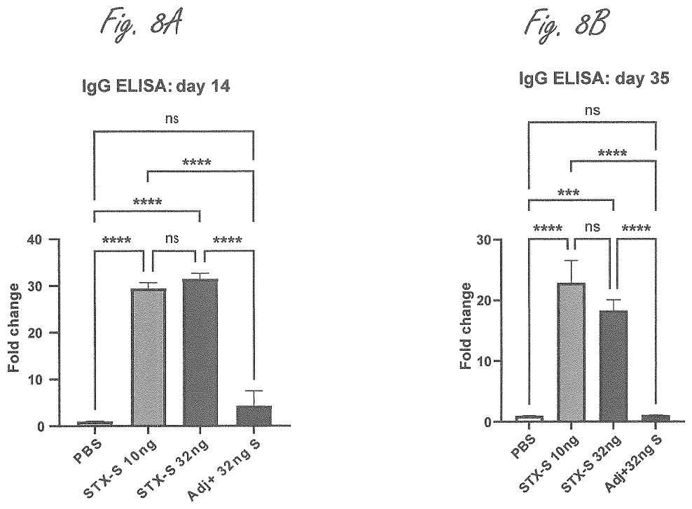

Turning to FIGS. 6A-C, exosomes were isolated from 293F cells that harbored the nucleocapsid-CD9 construct (FIG. 2A and FIG. 2B). FIG. 6A shows the size distribution of those exosomes from about 50 nm to about 270 nm, with a median of about 100-150 nm. FIG. 6B shows expression of nucleocapsid-containing fusion protein in the exosomes that were derived from the transduced 293F cells (lane 5). FIG. 6C demonstrates significant expression of spike-containing fusion protein on those exosomes as determined by spike flow cytometry. In one embodiment, expression of the nucleocapsid-CD9 fusion protein was put under control of the tet-inducible promotor. Here, the exosomes derived from the uninduced, but transduced host cells did not express nucleocapsid (see second curve from left at FIG. 6C).

In one embodiment, the exosomes expressing the spike-CD9 fusion protein (FIGS. 1A-B and 4A-C) were delivered to a subject by intramuscular injection according to a two-dose schedule (see, e.g., FIG. 7). Likewise in another set of experiments, the exosomes expressing the nucleocapsid-CD9 fusion protein (FIGS. 2A-B and 6A-C) were delivered to a subject by intramuscular injection according to the same two-dose schedule (see, e.g., FIG. 7).

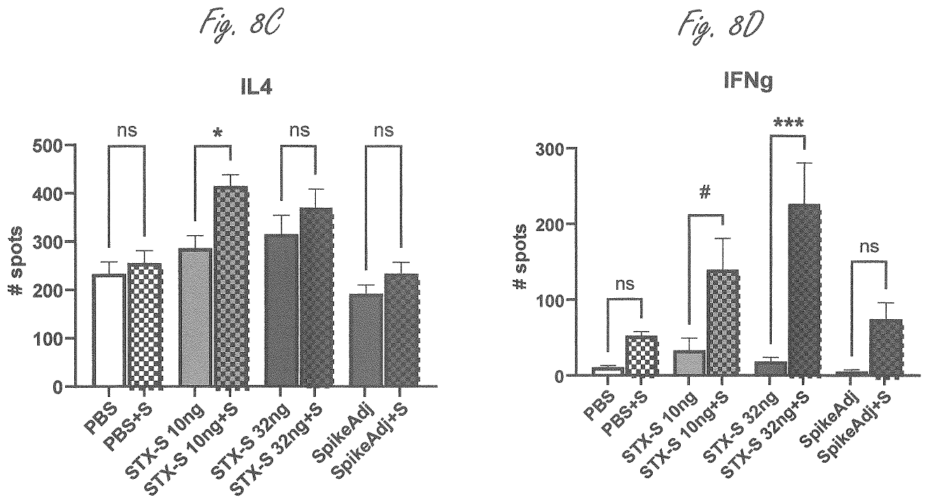

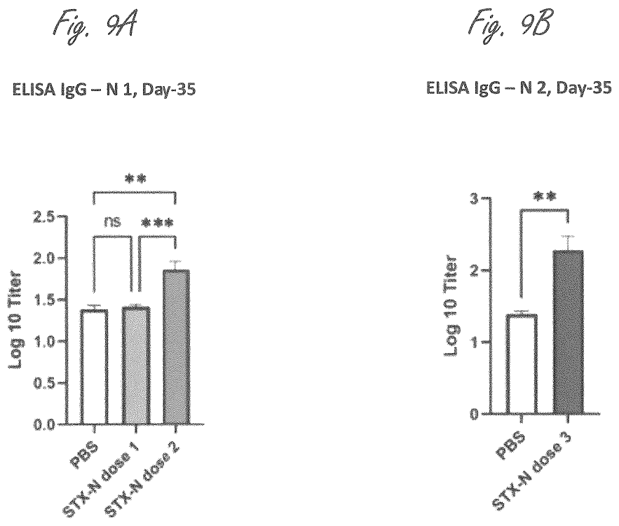

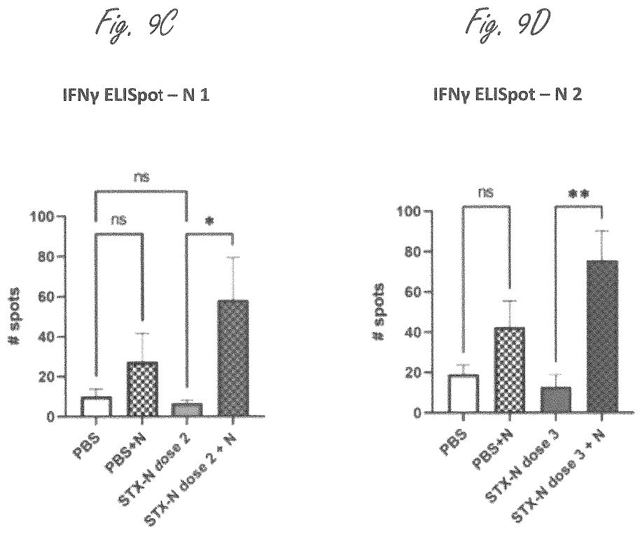

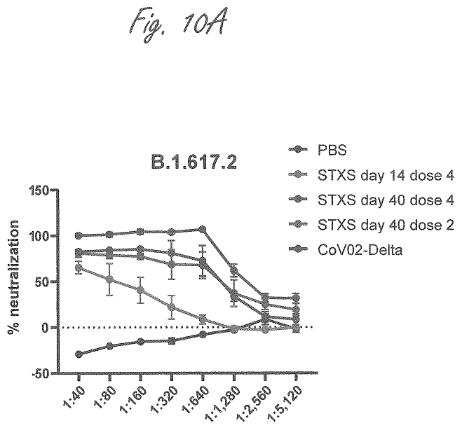

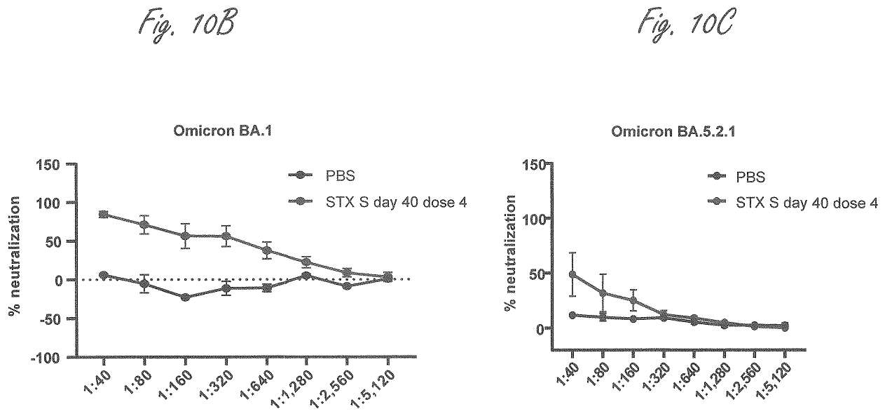

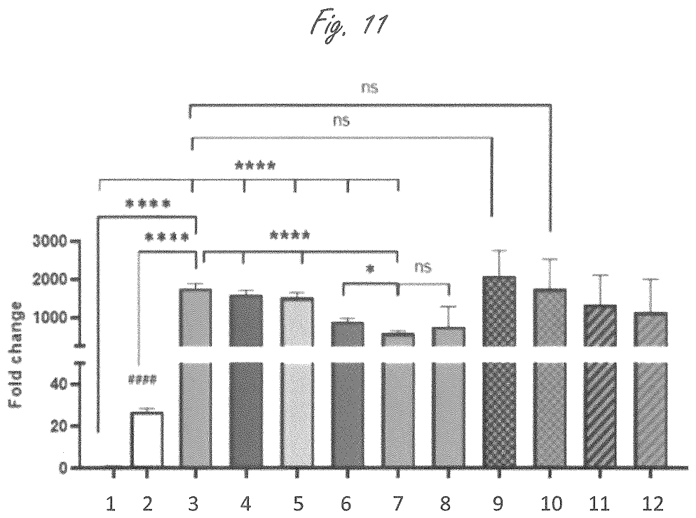

Turning to FIG. 7, at day-1, subject mice were administered by intramuscular injection 1× dose (i.e., 3E10 vesicles) or 10× dose (i.e., 3E11 vesicles) expressing either the spike-CD9 fusion protein or the nucleocapsid-CD9 fusion protein (710). At day-14, blood was collected from the administered mice and assessed for early humoral immune-responses (720) (see FIGS. 8A and 9A). At day-21, the subject mice received a second dose of the subject spike or nucleocapsid expressing exosomes (730). At day-35, blood was collected from the administered mice and assessed for humoral immune-responses, including antibody production (see FIGS. 8B and 9B) and neutralizing antibody production (740). At day-40, splenocytes were collected from the administered mice and assessed via ELISpot assay for cellular immune-responses (750) (see FIGS. 8C, 8D, 10A and 10B).

Turning to FIGS. 8A and 8B, immune responses to exosome-expressing COVID-19 spike antigen (STX-S) at the humoral level were induced in a subject as early as two weeks after the first injection (FIG. 8A) and persisting at least until day-35 post-injection (FIG. 8B). Furthermore, the robust and persistent anti-spike antibody response was elicited using nanogram amounts of spike protein expressed on exosomes and without the inclusion of an adjuvant. Here, for example, as little as 10 ng of exosome-expressed spike protein without adjuvant elicited a significant and useful antibody response as measured at day-14 and day-35. Also, 32 ng of exosome-expressed spike protein without adjuvant elicited a significant≥30-fold antibody response compared to 32 ng of purified spike protein (exosomeless) with adjuvant. It is generally known in the art that prior art spike protein vaccines are administered in microgram quantities and with adjuvant to generate an antibody response similar to that observed with nanogram quantities of the instant exosome-expressed spike protein antigen.

Turning to FIGS. 8C and 8D, immune responses to STX-S at the cellular level were induced in a subject as shown in ELISpot assays performed on splenocytes obtained at day 35-40. Here, a dose of as little as 10 ng of adjuvantless exosome-expressed spike antigen elicited in a subject a significant CD4+ T-cell response against spike protein as demonstrated in an IL4 ELISpot assay of subject splenocytes (FIG. 8C). Here also, doses of 10 ng and 32 ng of adjuvantless exosome-expressed spike antigen elicited in a subject a significant CD8+ cytotoxic T-cell response against spike protein as demonstrated in an IFNγ ELISpot assay of subject splenocytes (FIG. 8D).

Similar nanogram doses of adjuvantless exosome-expressing SARS-CoV-2 nucleocapsid protein also elicit useful, robust, and significant humoral and cell-mediated immune responses (FIGS. 9A-D). The inventors envision that any or most viral antigens (such as, e.g., influenza hemagglutinin, influenza neuraminidase, respiratory syncytial virus, and/or other viral glycoproteins or other protein or fragments thereof) expressed on the surface of exosomes according to the present disclosure will elicit humoral and/or cell-mediated immune response in the recipient subject.

Thus, in some embodiments, a low-dose immunogenic composition or vaccine is provided that contains an immunogenic dose inclusively between 1 ng and 1 μg viral antigen (e.g., SARS-CoV-2 spike or nucleocapsid), 1-900 ng, 1-800 ng, 1-700 ng, 1-600 ng, 1-500 ng, 1-400 ng, 1-300 ng, 1-200 ng, 1-100 ng, 1-90 ng, 1-80 ng, 1-70 ng, 1-60 ng, 1-50 ng, 1-40 ng, 5-100 ng, 5-90 ng, 5-80 ng, 5-70 ng, 5-60 ng, 5-50 ng, 5-40 ng, ≤100 μg, ≤50 μg, ≤25 μg, ≤20 μg, ≤10 μg, ≤1 μg, ≤900 ng, ≤800 ng, ≤700 ng, ≤600 ng, ≤500 ng, ≤400 ng, ≤300 ng, ≤200 ng, ≤100 ng, ≤90 ng, ≤80 ng, ≤70 ng, ≤60 ng, ≤50 ng, ≤40 ng, about 1 ng, about 1.5 ng, about 2 ng, about 2.5 ng, about 3 ng, about 3.5 ng, about 4 ng, about 4.5 ng, about 5 ng, about 6 ng, about 7 ng, about 8 ng, about 9 ng, about 10 ng, about 11 ng, about 12 ng, about 13 ng, about 14 ng, about 15 ng, about 16 ng, about 17 ng, about 18 ng, about 19 ng, about 20 ng, about 25 ng, about 30 ng, about 35 ng, about 40 ng, about 45 ng, about 50 ng, about 55 ng, about 60 ng, about 65 ng, about 70 ng, about 75 ng, about 80 ng, about 85 ng, about 90 ng, about 95 ng, about 100 ng, about 105 ng, about 110 ng, about 115 ng, about 120 ng, about 125 ng, about 130 ng, about 135 ng, about 140 ng, about 145 ng, about 150 ng, about 155 ng, about 160 ng, about 165 ng, about 170 ng, about 175 ng, about 180 ng, about 185 ng, about 190 ng, about 195 ng, about 200 ng, about 205 ng, about 210 ng, about 215 ng, about 220 ng, about 225 ng, about 230 ng, about 235 ng, about 240 ng, about 245 ng, or about 250 ng of viral antigen (e.g., SARS-CoV-2 spike or nucleocapsid).

Turning to FIGS. 9A and 9B, immune responses to exosome-expressing COVID-19 nucleocapsid antigen (STX-N) at the humoral level were induced in a subject and detected a day-35 post-injection (FIGS. 8A and 8B). Furthermore, the robust and persistent anti-spike antibody response was elicited using nanogram amounts of spike protein expressed on exosomes and without the inclusion of an adjuvant. Here, for example, as little as 3.2 ng of exosome-expressed nucleocapsid protein without adjuvant elicited a significant and useful antibody response as measured at day-35 (FIG. 9A, STX-N dose 2). Thus, in one embodiment, a complete immunization cycle (two i.m. injections) induced a significant 3-fold increase of IgG against SARS-CoV-2 nucleocapsid protein (N) over PBS for the dose of about 3 ng/injection (dose 2, FIG. 9A) up to a 10-fold at 10 ng/injection (dose 3, FIG. 9B).

Turning to FIGS. 9C and 9D, to characterize the T cell response to STX-N, antigen-specific T cell responses to nucleocapsid protein were measured by ELISpot assays performed on splenocytes obtained at day 40. Here, vaccination of subjects with STX-N elicited multi-functional, antigen-specific T cell responses against SARS-CoV-2 nucleocapsid protein at day 40 (after boost (2nd) injection). STX-N administration resulted in a potent IFNγ response (FIGS. 9C and 9D). Here, evaluation of IFNγ secreting cells in response to ex-vivo nucleocapsid protein stimulation showed a 6-fold increase in spleens immunized with STX-N (FIGS. 9C and 9D), suggesting a Th1-biased CD8+ T cell response. This response is of invaluable importance for the development of exosome-expressing immunogenic compositions with the potential to protect against a wide range of SARS-CoV-2 variants, since nucleocapsid protein is not a surface protein and otherwise most likely will only been presented to the immune cells once the infecting viri are processed inside the infected cells.

As in the humoral antibody response to nucleocapsid protein (FIGS. 9A, 9B), in this particular embodiment, the significant CD8+/IFNγ response to nucleocapsid protein in subjects was effected with administered doses of STX-N in the 3 to 10 ng range without adjuvant, which is on the order of three orders of magnitude lower than prior art protein subunit vaccines.

Turning to FIGS. 10A-C, the administration of nanogram quantities of STX-S to subjects elicited potent neutralization of both delta and omicron variants of SARS-CoV-2 induced by STX-S exosome injection. In one embodiment, day-40 sera from subjects administered about 3.2 ng STX-S per injection (dose 2, FIG. 10A) and day-14 and day-40 sera from subjects administered about 9.8 ng STX-S per injection (dose 4, FIG. 10A) were tested for neutralizing antibodies against SARS-CoV-2 Delta variant. Here, potent neutralizing activity was elicited by STX-S in each sample tested (FIG. 10A) comparable to SARS-CoV-2 delta positive vaccine sera. STX-S displayed dose-dependent neutralization of the virus, estimated by the ability to protect infected cells from the virus-induced cytopathic effects (compare dose 4 to dose 2 at day-40 sera, FIG. 10A).

Thus, according to one embodiment, the STX-S engineered exosome vaccine induced in subject(s) neutralizing antibodies against delta spike by day 14 (after a single i.m. injection). By day 40, ˜4 weeks post STX-S boost, robust neutralization was observed in all subjects regardless of dose (FIG. 10A). Here, one injection with about 9 ng of spike by STX-S (dose 4, FIG. 10A) delivered about 65-75% neutralization against a delta variant. Moreover, full immunization (i.e., initial injection and at least one booster, or two i.m. injections) resulted in about 80-85% of neutralization against delta variant at a dose of about 3-9 ng of spike delivered by STX-S exosomes.

The delta-variant neutralization elicited by the administration of STX-S in subjects induced a response comparable to the human control plasma (CoV02-delta, plasma from a patient immunized with Moderna's mRNA vaccine, with breakthrough delta infection), with a complete neutralizing response at higher dilutions (see, e.g., the 1:320, STX-S dose 4 day-40) (FIG. 10A).

Additionally, turning to FIGS. 10B and 10C, day-40 sera samples from subjects given the about 9-10 ng STX-S dose (here, the spike protein of the STX-S is of a delta variant—STX-Sa) were also tested for neutralizing antibodies against SARS-CoV-2 Omicron variants (Omicron BA.1 and BA.5.2.1). As shown in FIGS. 10C and 10D, a strong cross neutralization was observed for the sera obtained from STX-S^{δ}-treated subjects, achieving, in this embodiment, about 84% neutralization of Omicron BA1 variant (FIG. 10B) and a range between 16% to 97% for Omicron BA5 variant (FIG. 10C), after full immunization (i.e., at least 2 i.m. injections) with a dose of about 9-10 ng of delta spike delivered by STX-S exosomes. Thus, in some embodiments, the administration of an immunogenic composition containing single variant spike STX-S exosomes provides some level of protective immunity against other SARS-CoV-2 variants.

Turning to FIG. 11, the antibody response to spike from STX-S in the murine model persists for at least about 6 months and is expected to persist for more than six months. As shown in FIG. 11, mice dosed with 10 ng STX-S and boosted with 7 ng STX-S or 3 ng STX-S exhibit a strong IgG response to spike antigen at least 166 days after the initial dose.

In addition to their superior immune-response elicitation, both the spike-exosome vaccine and the nucleocapsid-exosome vaccine presents several other advantages over currently available vaccines. First, the subject exosome-based vaccines deliver antigen through an entirely endogenous, autologous lipid bilayer, that can be easily integrated into the host cell membrane and facilitate engineered antigen presentation to immune cells. Membrane bound antigen can easily be presented to the circulating immune cells to quicky activate a response, while free antigen contained inside the exosome can additionally be processed by the lysosomal system and activate the cytotoxic T-lymphocyte response. Thus, utilization of a natural delivery system promotes efficient delivery and response compared with synthetic lipid nanoparticle technology.

Second, the spike and nucleocapsid exosome vaccines are protein-based vaccines, making the antigen readily available to the subject immune system without translation in a host cell as is required for RNA-based vaccines. Incomplete translation, and/or incorrect folding of mRNA encoding Spike or nucleocapsid protein limits the amount of available antigen after vaccination, making the immune response extremely variable and with reduced efficacy.

Third, the subject exosome-based spike and nucleocapsid fusion protein vaccines do not require an adjuvant or synthetic lipid nanoparticle (LNP) for delivery and immune response. Traditional protein-based vaccines require adjuvants (such as, e.g., aluminum salt and the squalene oil-in-water emulsion systems MF59 (Novartis) and AS03 (GlaxoSmithKline)) (see, e.g., Wong, S. S. and R. J. Webby, “Traditional and new influenza vaccines,” Clin Microbiol Rev, 2013. 26(3). p. 476-92) to augment and orchestrate immune responses and influence affinity, specificity, magnitude and functional profile of B and T cell responses. Current mRNA vaccines do not use an adjuvant, which combined with the need for mRNA translation prior to antigen availability, may also reduce long term efficacy. Current approved COVID vaccines use LNPs to deliver the mRNA or adjuvants for the proteins and undesirable side effects have been reported.

Fourth, the subject exosomal spike fusion protein and nucleocapsid fusion protein Immunogenic compositions exhibit greater immunogenic efficacy at a significantly lower, i.e., three orders of magnitude lower, dose than currently available LNP mRNA and protein vaccines. For example, clinical approved protein candidate vaccines use approximately 5 ug to 25 ug of antigen in conjunction with an adjuvant to induce immunization (see, e.g., Formica, et al., “Different dose regimens of a SARS-CoV-2 recombinant spike protein vaccine (NVX-CoV2373) in younger and older adults: A phase 2 randomized placebo-controlled trial,” PLoS Med, 2021. 18(10): p. e1003769; Sun et al., “Development of a Recombinant RBD Subunit Vaccine for SARS-CoV-2,” Viruses, 2021. 13(10); Worzner et al., “Adjuvanted SARS-CoV-2 spike protein elicits neutralizing antibodies and CD4 T cell responses after a single immunization in mice. EBioMedicine, 2021. 63: p. 103197; and references therein. Here, the immunology data from subjects immunized with either the spike fusion-exosome or the nucleocapsid fusion-exosome elicits a complete immunization that is correlated with high antibody levels, strong viral neutralization, broad multi-variant activity, and both B and T cell memory with approximately 1/1000th of the protein antigen amount (i.e., nanogram amounts versus microgram amounts) currently being administrated in current clinically approved vaccines.

Fifth, an additional limitation of the available vaccines is the need of refrigeration and short shelf life of the products at higher temperatures. Because of their high stability at physiological pH and temperature, exosomes, including display exosomes, can be stored at 4° C. for longer times. The spike fusion or nucleocapsid fusion-expressing exosome composition can also be lyophilized for long term storage at convenient temperature (see, e.g., U.S. Pat. App. No. 2017/0360842 A1).

Thus, here, a strong CD8+ T cell response as well as a strong B cell response, as demonstrated by IgG production and potentially neutralizing antibodies, was induced when spike protein or nucleocapsid protein was delivered by exosomes. While not wishing to be bound by theory, this result could be explained by the role of extracellular vesicles in intercellular communication and antigen presentation. In particular, numerous copies of the spike protein (FIGS. 5A and B) or nucleocapsid protein may be present on the vesicle surface, facilitating the crosslinking to B-cell receptors. Moreover, spike proteins in extracellular vesicle-based vaccines could indirectly activate B cells and CD8(+) T cells through the antigen cross-presentation.

It is further envisioned that the subject fusion protein-expressing exosomes may be engineered to express antigens of interest to target new COVID variants. The antigen of interest could easily be swapped and adapted to the needs. Antigens could be expressed as fusion proteins (e.g., chimeras) with, e.g., exosome expression or display domains fused to antigen domains to enable display of antigens on the exosome for delivery to the host subject immune system.

It is further envisioned that the exosomes could be engineered to selectively target organs or tissues of interest and allow safe and targeted delivery of antigens to specific immune subsystems to elicit specific types of responses in the subject.

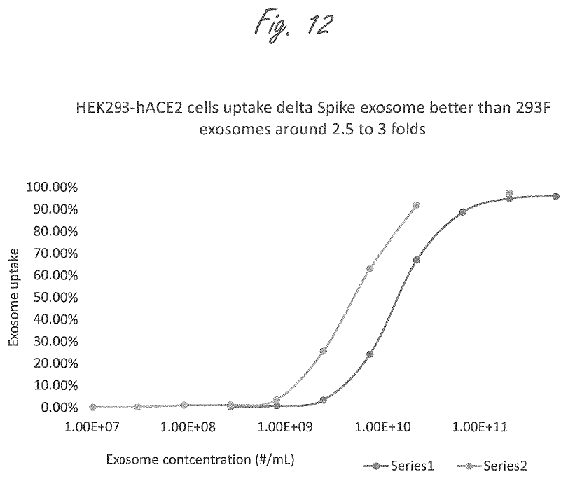

Here, turning to FIG. 12, HEK293-hACE2 cells were seeded into 24-well plates and contacted with from about 1E7 to 1E12 exosomes per mL in reduced serum medium at about 37° C. for about 3.5 hr. In one series (series 2), the exosomes expressed spike-CD9. In another series (series 1), the exosomes did not express spike-CD9 or spike in any other form. Here, HEK293-hACE2 cells demonstrated better uptake of spike-expressing exosomes at about 2.5 to 3-fold lower concentration relative to “wildtype” 293F wt exosomes. See also Table 1.

TABLE 1
Uptake of Wildtype and Spike-Expressing Exosomes by HEK293-hACE2 Cells

| 293T Exosome @ | Wt 293T | Viability of wt 293T | Delta Spike | Viability of Delta Spike |
|---|---|---|---|---|
| #/m, 3x lots (Lot. 18, | Exosome | exosome-uptaken | Exosome uptake + | exosome-uptaken cells |
| 2.43E12, 2.6 mg/mL) | uptake+ | cells (BD FVS780−) | (GG1-1) | (BD FVS780−) |
| 6E+11 | 96.07% | 84.68% | | |
| 2E+11 | 95.09% | 96.87% | 97.6% | 85.63% |
| 6.7E+10 | 88.9% | 87.1% | 86.61%* | 81.75%* |
| 2.2E+10 | 67.04% | 85.08% | 92.06% | 85.86% |
| 7.4E+9 | 24.29% | 86.24% | 63.24% | 86.02% |
| 2.5E+9 | 3.5% | 87.05% | 25.7% | 86.9% |
| 8.2E+8 | 0.92% | 85.5% | 3.66% | 87.22% |
| 2.7E+8 | 0.39% | 86.5% | 1.29% | 85.5% |
| 9.1E+7 | | | 1.26% | 86.28% |
| 3E+7 | | | 0.36% | 85.77% |
| 1E+7 | | | 0.21% | 84.93% |
| PBS | 0.66% | 85.03% | | |

Thus, spike-expressing exosomes may be used to deliver any drug cargo to lung or other tissues that express a spike receptor, such as ACE2. Furthermore, this data further evidences the fact that the disclosed exosomes expressing a recombinant fusion protein on their surface preserve the native structure of the various components of the fusion protein, e.g., the spike protein polypeptide has sufficient native structure to engage a natural target i.e., ACE2.

### EXAMPLES

The pandemic emergency has brought to light the need for potent, reliable, and broader vaccine to contrast the ever-changing viruses and stop the virus transmission. While the COVID19 mRNA vaccine played an important role during the emergency in reducing the casualties of the SARS-CoV-2 infections, its reduced ability to protect against new VOCs, together with the need of multiple injections to sustain the protection, pushes the scientific community to look for new approaches.

SARS-CoV-2 did teach us that a successful vaccine will produce a strong antibody response, with neutralizing antibody and a strong T-cell response able to contrast the viral infection broader and timely, with a minimum number of injections. Those criteria could be met by exosome-based vaccines together with a “multivalent” approach.

Here, the following examples have shown that exosomes can be used to deliver viral proteins for immunization: the disclosed STX platform generated two vaccine candidates (STX-S and STX-N), that independently, and in combination (STX-S+N) induced a strong immune response against two SARS-CoV-2 proteins (spike and nucleocapsid) with a single shot, in two different animal models by delivering nanograms of proteins on the surface of the exosomes. No adjuvant was needed, 100× less protein was used and no competition between proteins was observed.

The “multivalent” or “combination” vaccines have multiple advantages. For start, they require fewer injections, which would engage the population positively, increasing as a result the percentage of vaccinated population with broader epidemiological benefits. Consequently, less injections means lower overall budgeting and cost. Of course, there are limitations that need to be taken in consideration. First, selection of antigen: it is crucial that the selected antigen does retain strong immunogenicity once in combination, that has a hopefully conserved sequence across species and ideally lower mutagenesis rate. Of them all, immunogen interference is critical: minimal to no competition should be observed. As per the exemplified STX-S+N, the multivalent vaccine shows the same strength of efficacy as the single product, with thousands fold increase in antibody load. Additionally, STX-S+N elicited both a quantitative and qualitative immune response: consistent increase in amount of antibody produced, presence of protection as delineated by neutralizing antibodies and engagement of T cells were all observed in response to the administration of the disclosed exosome vaccine STX-S+N. Importantly, no deleterious side effects were recorded: both mice and rabbits showed no changes in weight or blood tests, and no alteration at the tissue levels, suggesting an overall safe profile.

The data presented herein suggest that exosomes are ideal vehicles for vaccination: they can safely deliver the antigen of interest (exogenous protein), in a way mimicking the natural viral infection. Exosome based vaccines constitute an innovative approach for an efficient virus-free, human-derived vaccine design. Yoo et al (“Possibility of exosome-based coronavirus disease,” 2019 vaccine (Review). Mol Med Rep, 2022. 25(1)) observed that exosomes or extracellular vesicles at large could support the vaccination needs beyond traditional strategies: compared to viral or vector methods, exosomes are not immunogenic themselves, but are carrier of a protein that retains the original conformation, tridimensional structure and modifications, all embedded in the lipid biolayer of its membrane and ready to be efficiently presented as such to the immune system.

The strong T cell response initiated by STX-S+N administration is clinically relevant. Keeton et al (“T cell responses to SARS-CoV-2 spike cross-recognize Omicron,” Nature, 2022. 603(7901): p. 488-492) observed that while neutralizing antibody might not recognize the new VOC, the T cell population is able to cross react with them and confer protection. Here, a spike and a Ncap specific T-cell response was observed: the highly conserved Ncap can further increase and broaden the efficacy of the STX vaccine, engaging additional immune response that is not compromised by a surface protein naturally mutating. This suggests that where the new variant of concern (VOC) escape the barrier of neutralizing antibodies, the Tcell response could cross-reactive and limiting the infection.

Other groups have reported a multivalent vaccine for SARS-CoV-2 using a more viral approach coexpressing the SARS-CoV-2 N and S proteins on VSV virus (e.g., O'Donnell, K. L., et al., “Protection from COVID-19 with a VSV-based vaccine expressing the spike and nucleocapsid proteins,” Front Immunol, 2022. 13: p. 1025500) or by adenovirus (e.g., Dangi, T., et al., “Combining spike- and nucleocapsid-based vaccines improves distal control of SARS-CoV-2,” Cell Rep, 2021. 36(10): p. 109664). Interestingly, use of a multiprotein vaccine broadens the efficacy to distal organs, reducing the viral charge not only on the respiratory system but to the distant brain, as well (see, e.g., Matchett, W. E., et al., “Cutting Edge: Nucleocapsid Vaccine Elicits Spike-Independent SARS-CoV-2 Protective Immunity,” J Immunol, 2021. 207(2): p. 376-379). Nucleocapsid specific immunity plays a dispensable role during a SARSCoV-2 infection. While antibody responses can block the initial entry of the virus at proximal sites of infection, it is the T cell response that plays a critical role in controlling propagation of infection, second-round infections and subsequent viral dissemination to distal sites, providing a synergistic antiviral effect by killing virally infected cells, curtailing further the dissemination of the virus to the peripheral organs.

Disclosed is a single-dose dual-antigen vaccine with efficacy against multiple SARS-CoV-2 VOC, that has broader immune capability, and could be used as a boost of the existing immunity generated by previously approved vaccines. The disclosed StealthX vaccine technology (STX), which uses exosomes to deliver nano gram quantities of viral antigen to elicit strong, broader immune response without any adjuvants.

### Example 1: Cell Lines

Human embryonic kidney 293 T cells (293T) were purchased from ATCC (CRL-3216). 293T cells were maintained in culture using Dulbecco's Modified Eagle Medium (DMEM), high glucose, Glutamax™ containing 10% fetal bovine serum. 293T cells were incubated at 37° C./5% CO₂. FreeStyle™ 293F cells (Gibco, 51-0029) were purchased from ThermoFisher, Waltham, MA. 293F cells were used as a parental cell line to generate spike SARS-CoV-2 delta spike expressing stable cell lines: Stealth X-Spike cells (STX-S). 293F and STX-S cells were maintained in a MULTITRON incubator (Infors HT, Sulzemoos, DE) at 37° C., 80% humidified atmosphere with 8% CO₂ on an orbital shaker platform rotating at 110 rpm.

### Example 2: Lentiviral Vectors

Lentiviral vectors for expression of SARS-CoV-2 spike (Delta variant B.1.617.2, NCBI accession #OX014251.1, available on Aug. 15, 2023, at www.ncbi.nlm.nih.gov/nuccore/OX014251.1) and SARS-CoV-2 nucleocapsid (NCBI accession #OP359729.1, available on Aug. 15, 2023, at www.ncbi.nlm.nih.gov/nuccore/OP359729.1) were designed and synthesized from Genscript together with the two packaging plasmids (pMD2.G and psPAX2). Lentiviral particles for transduction were generated by transfecting 293T cells with pMG.2 (Genscript, GenScript Biotech, Piscataway, NJ), psPAX2 (Genscript) and STX-S_pLenti (Genscript) expressing spike or STX-N_pLenti (Genscript) expressing nucleocapsid at a ratio of 5:5:1 using Lipofectamine™ 3000 (ThermoFisher Scientific, Waltham, MA) according to the manufacture's instruction. Spike and nucleocapsid lentiviral particles were collected at 72 hours post transfection and used to transduce 293F parental cells to generate STX-S and STX-N respectively.

### Example 3: Flow Cytometry

Standard flow cytometry methods were applied to measure the spike SARS-CoV-2 protein expression on STX cell surface. In brief, 250K STX cells were aliquoted, pelleted and resuspended in 100 uL eBioscience™ Flow Cytometry Staining Buffer (ThermoFisher). Cells were incubated at room temperature (RT) for 30 min protected from light in the presence of anti-spike antibody (Abcam, clone 1A9, ab273433, Abcam, Cambridge, UK) or anti-nucleocapsid (Abcam, ab281300) labeled with Alexa Fluor®-647 (Alexa Fluor®647 Conjugation Kit (Fast)-Lightning-Link®(Abcam, ab269823) according to the manufacturer's protocol. Following incubation, STX cells were washed with eBioscience™ Flow Cytometry Staining Buffer (ThermoFisher, cat No 00-4222-57), resuspended in PBS and analyzed on the CYTOFLEX S flow cytometer (Beckman Coulter, Brea, California). Data was analyzed by FLOWJO (Becton, Dickinson and Company, Franklin Lakes, NJ).

### Example 4: Cell Sorting

Cell sorting was performed at the Flow Cytometry Facility at the Scripps Research Institute (San Diego, CA). To enrich the spike positive population, STX-S cells were stained as described above for flow cytometry and then subjected to cell sorting (Beckman Coulter MOFLO ASTRIOS EQ) to generate pooled STX-S. The pooled STX-S were used in the examples presented herein unless specified otherwise.

### Example 5: STX Exosome Production

STX-S and STX-N cells were cultured in FREESTYLE media (ThermoFisher, 12338018) in a MULTITRON incubator (Infors HT) at 37° C., 80% humidified atmosphere with 8% CO₂ on an orbital shaker platform. Subsequently, cells and cell debris were removed by centrifugation, while microvesicles and other extracellular vesicles larger than ˜220 nm were removed by vacuum filtration. Next, exosomes were isolated using filtration and size exclusion. Briefly, supernatant was subjected to concentrating filtration against a Centricon Plus-70 Centrifugal filter unit (Millipore, UFC710008, Millipore Sigma, St. Louis, MO), then subjected to size exclusion chromatography (SEC) using a qEV original SEC column (Izon, SP5, Izon Science, Christchurch, NZ).

### Example 6: Nanoparticle Tracking Analysis

Exosome size distribution and concentration were determined using ZetaView Nanoparticle Tracking Analysis (Particle Metrix, Inning am Ammersee, DE) according to manufacturer instructions. Exosome samples were diluted in 0.1 μm filtered 1× PBS (Gibco, 10010072) to fall within the instrument's optimal operating range.

### Example 7: Protein Expression

Detection of SARS-CoV-2 spike and nucleocapsid proteins in cell lysate and exosomes used a JESS capillary protein detection system (ProteinSimple, San Jose, CA). Samples were lysed in RIPA buffer (ThermoFisher Scientific, 8990) supplemented with protease/phosphatase inhibitor (ThermoFisher Scientific, A32961), quantified using the BCA assay (ThermoFisher Scientific, 23227) and run for detection. To detect spike protein, the separation module 12-230 kDa was used following manufacturers protocol. Briefly, 0.8 μg of sample and protein standard were run in each capillary, probed with anti-mouse Ms-RD-SARS-COV-2 (MAB105401, 1:10 dilution; R&D Systems, Minneapolis, MN) or anti-rabbit Nucleocapsid (NBP3-00510, 1:100 dilution; Novus Biologicals, Centennial, CO) followed by secondary antibody provided in JESS kits (HRP substrate used neat).

### Example 8: TEM Imaging for Characterization of STX Exosome Morphology

STX-S and STX-N exosome samples were negatively stained onto copper grids with a carbon film coating and imaged by TEM at the Electron Microscopy Core Facility at UC San Diego (San Diego, CA). Briefly, samples were treated with glow discharge, stained with 2% uranyl acetate and dried before imaging. Grids were imaged on a JEM-1400 Plus (JEOL Ltd, Japan) at 80 kV and 48 uA. Images were taken at from 12K-80K magnification at 4 k×4 k pixels of resolution.

### Example 9: CD81 Bead-Assay

STX-S or 293F parental exosomes were mixed with anti-CD81 labeled magnetic beads for 2 hours at room temperature (RT) (ThermoFisher, 10622D) and washed twice with PBS using a magnetic stand. Next, the bead-exosomes were incubated with either direct conjugated AlexaFluor 647 anti-spike (see above flow cytometry), FITC anti-CD81 antibody (BD Biosciences, 551108, Franklin Lakes, NJ), or FITC Mouse IgG, κ isotype control (BD Biosciences, 555748) for 1 hour at RT followed by two PBS washes. 293F exosomes were used as a negative control for spike expression and the isotype antibody was used as a negative control for CD81 expression. Samples were analyzed on a CytoFlex S (Beckman Coulter) flow cytometer and data was analyzed by FLOWJO.

### Example 10: Mouse Studies

To examine the efficacy of STX exosomes, age matched BALB/c mice (female, 8-10 wks old) were anesthetized using isoflurane and received bilateral intramuscular injection (50 μl per leg, total 100 μl) of either 1) PBS 2) STX-S, 3) STX-N or 4) STX-S+N exosomes. Booster injection was performed at day 21. Mice were monitored closely for changes in health and weight was recorded biweekly. Blood collection was performed at day 14 and day 35. Blood (˜50-500 μl) was collected from the submandibular vein and processed for plasma isolation after centrifugation at 4000 rpm for 5 min at 4° C. For comparison study, mice were injected with equal amounts of SARS-CoV-2 protein delivered as 1) soluble protein in conjugation with adjuvant (Alhydrogel, 100 μg/dose, vac-alu-250, InvivoGen, San Diego, California), or 2) STX exosome. Blood was collected 2 weeks after injection and tested for IgG against SARS-CoV-2. Timeline of mouse study is outlined in FIG. 7B. Mouse tissues (brain, salivary gland, heat, lung, liver, spleen, kidney, gastro-intestinal tract (GI) and skeletal muscle (site of injection) were collected and fixed in fixed in 10% Neutralized Formalin. Sections were stained with hematoxylin and eosin and analyzed for alterations.

### Example 11: Rabbit Studies

To evaluate the potential toxicity and host immune response of the subject STX-S+N vaccine, age matched rabbits (male/female, New Zealand White, 2.5-3.0 kg) received intramuscular (IM) injection of the intended human dose (10-200 ng total protein in 0.5 mL) of the STX-S+N vaccine. Control animals received phosphate-buffered saline (PBS). Booster injection was performed at day 14. Rabbits were monitored closely for changes in health and weight recorded. Blood collection was performed weekly on day 0, 7, 14, 21, 28 and processed for plasma isolation after centrifugation at 4000 rpm for 5 min at 4° C. Timeline of rabbit study is outlined in FIG. 7C. Rabbit tissues were collected at terminal (day 28), fixed in 10% Neutralized Formalin and processed for pathological alterations.

### Example 12: IgG ELISA

Mouse and Rabbit IgG antibodies against SARS-CoV-2 spike or nucleocapsid were measured by enzyme-linked immunosorbent assays (ELISA) using precoated ELISA plates (IEQ-CoV-S-RBD-IgG and IEQ-CoV-N-IgG, RayBiotech, Peachtree Corners, GA) according to the manufacturer's instructions, at room temperature (RT). Briefly, mouse plasma samples were diluted in sample buffer (RayBiotech) and added to antigen-coated wells, in triplicates, and incubated at RT for 2 hours on a shaker (200 rpm). Commercially available antibody against Spike (S1N-S58, Acro Biosystems, Newark, Delaware) or Nucleocapsid (NUN-S47, Acro Biosystems) was used as positive controls. Plates were washed 3 times with wash buffer, incubated for 1 h at RT with HRP-conjugated goat anti-mouse secondary antibodies (115-035-003, dilution 1:5000, Jackson ImmunoResearch) or anti-rabbit (111-035-003, dilution 1:5000, Jackson ImmunoResearch, West Grove, PA) diluted in assay buffer (RayBiotech). After 3 washes, plates were developed using TMB substrate (RayBiotech). After 15 min incubation, reaction was stopped by adding STOP solution and absorbance at 450 nm was recorded using a BIOTECK Gen5 plate reader (Agilent Technologies, Santa Clara, California). Endpoint titers were calculated as the dilution that emitted an optical density exceeding 4× the PBS control group.

### Example 13: Neutralizing Antibodies Against DELTA SARS-CoV-2

Vero E6 cells were used to evaluate the neutralization activity of the test-items against a replication competent SARS-CoV-2 delta variant (B.1.617.2). Samples were pre-incubated with the virus for 1 hour at 37° C. before addition to cells. Following pre-incubation of plasma/virus samples, cells were challenged with the mixture. Samples were present in the cell culture for the duration of the infection (96 h), at which time a Neutral Red uptake assay was performed to determine the extent of the virus-induced cytopathic effect (CPE). Prevention of the virus-induced CPE was used as a surrogate marker to determine the neutralization activity of the test-items against SARS-CoV-2. Test-items were evaluated in duplicates using two-fold serial dilutions starting at a 1:40 dilution (eight total dilutions). Control wells included “CoV02-Delta” and GS-441524, tested in singlet data points on each plate. “CoV02-Delta” is convalescent plasma from an individual who previously received two doses of the Moderna COVID-19 vaccine (Moderna, Cambridge, MA) before infection with the Delta variant. GS-441524 is an antiviral from Gilead Sciences (Foster City, California). CoV-01 is plasma from a patient who received two doses of the Moderna covid-19 vaccine with no prior history of SARS-CoV-2 infection. NT50 values of the test-items were determined using GraphPad Prism software (GraphPad Software, Boston, MA).

### Example 14: Neutralizing Antibodies Against Omicron (BA.1 and BA.5.2.1) SARS-CoV-2

Neutralization assays were performed using anti-NP immunostaining (Omicron BA.1 and BA.5.2.1). Briefly, samples were pre-incubated with the virus for 1 h at 37° C. before addition to Vero E6 cells. Following incubation, media was removed, and then cells were challenged with the SARS-CoV-2/test-item pre-incubated mix. The amount of viral inoculum was previously titrated to result in a linear response inhibited by antivirals with known activity against SARS-CoV-2. Cell culture media with the virus inoculum was not removed after virus adsorption, and test-items and virus were maintained in the media for the duration of the assay (48 hours). Subsequently, the extent of infection was monitored by incubating cells with a monoclonal test-item against the SARS-CoV-2 nucleocapsid (NP). The amounts of the viral antigen in infected cells were estimated after incubation with horseradish peroxidase conjugated polyclonal test-items against human IgG (HRP-goat anti-mouse IgG). The reaction was monitored using a colorimetric readout (absorbance at 492 nm). Test-items were evaluated in duplicates using two-fold serial dilutions starting at a 1:40 dilution. Control wells included GS-441524 (Gilead Sciences), tested in singlet data points on each plate.

### Example 15: Splenocyte Isolation

Spleens were processed for single cell isolation by mechanical disruption of spleen pouch using a syringe stopper and passage through a 0.040 mm mesh size nylon cell strainer to remove tissue debris. Erythrocytes were lysed using ammonium chloride potassium (ACK) buffer (A1049201, ThermoFisher), splenocytes were collected by centrifugation at 300×g for 5 min. Cellular pellet was resuspended in completed RPMI 1640 media (FG1215, Millipore Sigma Aldrich).

### Example 16: ELISPOT

splenocytes were isolated by mechanical disruption of spleen pouch and seeded at a concentration of 5E5 cells/well and incubated for 24 hOURS in the presence or absence of 10 μg/ml of SARS-CoV-2 Spike (S1N-C52H4, AcroBiosystems, Newark, Delaware) or Nucleocapsid (NUN-C5227, AcroBiosystems). Commercially available ELISPOT plate for evaluation of IL-4 (MuIL4, Immunospot, Cellular Technology Limited, Shaker Heights, OH), and IFNg (MuIFNg, Immunospot, Cellular Technology Limited; cat #3110-4APW-10, rabbit, MabTech, Cincinnati, OH) were used. Assay was performed according to manufacturer's guidelines. Plates were analyzed using the ELISPOT reader S6ENTRY (Immunospot, Cellular Technology Limited).

### Example 17: ELISA for Protein Quantification

Spike protein level on exosomes was measured by ELISA using precoated ELISA plates (ELV-COVID19S1, RayBiotech) according to the manufacturer's instructions, at RT. Nucleocapsid level on exosomes was measured by ELISA using precoated ELISA plates (Legend Max SARS-CoV2 Nucleocapsid protein ELISA Kit, 448007, BioLegend, San Diego, California) according to the manufacturer's instructions, at RT. Briefly, samples and standards were loaded to the precoated plate, and incubated 2-2.5 hours at RT on a shaker (200 rpm). Plates were washed and incubated with biotin-conjugated detection antibody for an hour at RT, followed by 45 minutes incubation in streptavidin solution. After washes, plates were developed using TMB substrate. After 30 minutes incubation, reaction was stopped by adding STOP solution and absorbance at 450 nm was recorded using a BIOTECK Gen5 plate reader (Agilent). For nucleocapsid, after 10 min incubation in TMB substrate, absorbance was recorded at 450 nm and 570 nm. For analysis, the absorbance at 570 nm can be subtracted from the absorbance at 450 nm, and optical density (OD) used to build a standard curve.

### Example 18: Pathology

Mouse tissues (brain, salivary gland, heat, lung, liver, spleen, kidney, gastro-intestinal tract (GI) and skeletal muscle (site of injection) were collected and fixed in fixed in 10% Neutralized Formalin. Sections were stained with hematoxylin and eosin and analyzed for alterations.

### Example 19: Statistical Analysis

Data were analyzed using Excel and GraphPad Prism 9.1 and shown as mean±sem. 1-way ANOVA with post-hoc correction for multiple comparisons or 2-tailed t-test were applied as needed.

### Example 20: SARS-CoV-2 Protein Expression on the Surface of STX Producing Cells and Exosomes

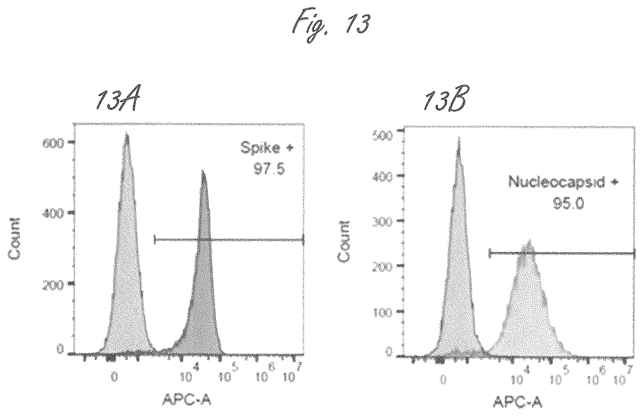

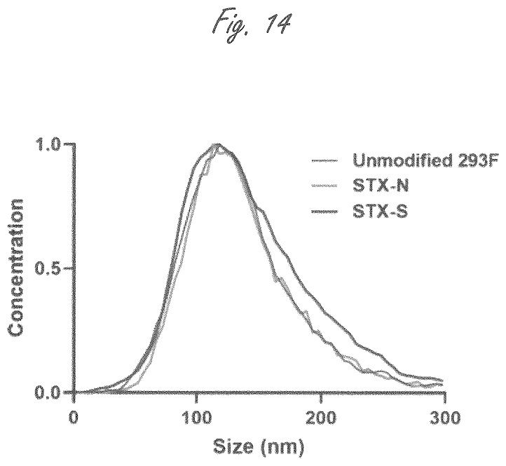

STX cells were generated by lentiviral transduction and expression of SARS-CoV-2 proteins on cells surface was evaluated by flow cytometry (FIG. 13). As shown, STX cells showed>95% increased expression of spike (FIG. 13A) and nucleocapsid (FIG. 13B) compared to parental 293F cells.

STX exosomes were purified from the engineered 293F cell culture supernatant using a lab-scale purification technique as described herein (Example 5). The purified STX-S and STX-N exosomes showed an expected average diameter of 144.6 nm and 140.4 nm (FIGS. 4A, 6A, and 14) and an expected polydispersity index (PDI) of <0.2 (0.152 and 0.129, FIG. 14), respectively.

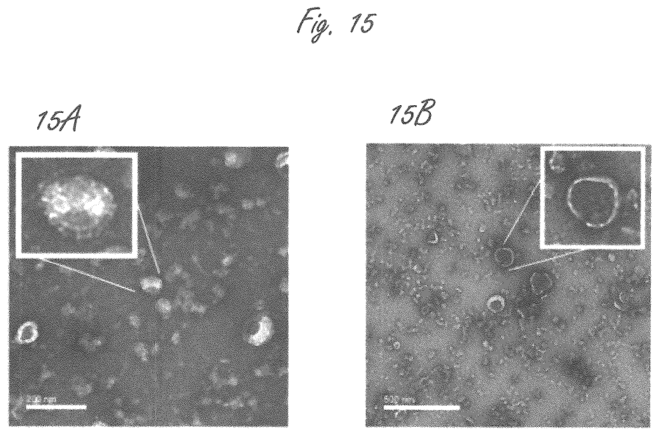

STX exosomes were analyzed by TEM imaging. As shown in FIG. 15A-B, typical exosome size and morphology were observed with round, smooth nano particles detected that had a visible lipid bilayer. Importantly, spike protrusions were visible on the surface of the STX-S nanoparticles indicating the presence of spike protein on exosomes (FIGS. 5A, 5B, and 15A). For Nucleocapsid, a characteristic lipid bilayer was observed, which resulted thicker than naïve exosomes, suggesting an accumulation of particles in the exosome membrane (FIG. 15B).

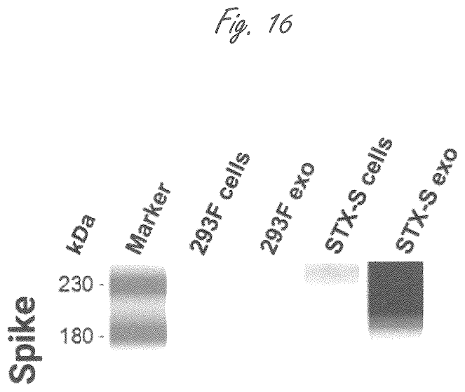

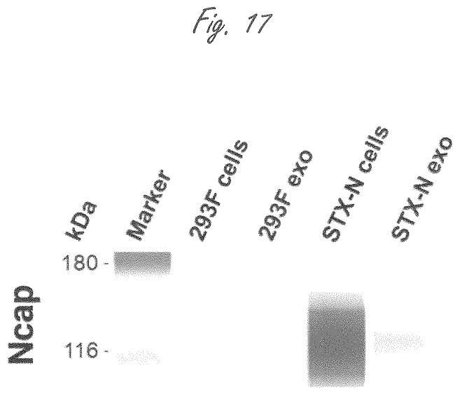

Spike and Nucleocapsid expression were validated on cell lysates and exosomes using Protein Simple's Jess automated Western blot as described herein. Spike protein was detected in both STX-S cells and exosomes, with enrichment of spike protein in the exosome samples (FIGS. 4B and 16). STX-N engineered cells and exosomes expressed abundant levels of Nucleocapsid (FIGS. 6B and 17). Further, SARS-CoV-2 protein spike and Ncap were detected on exosome membrane using a bead-based CD81 assay, with more >75% expression together with exosome specific marker CD81.

The concentration of Spike antigen in STX-S exosomes and Nucleocapsid antigen in STX-N exosome was further quantified by ELISA. A final STX-S prep of 1×10\hat{ }12 (1E12) exosomes/mL contains on average 253.77 ng of Spike, while a final STX-N prep of 1×10\hat{ }12 (1E12) exosomes/mL contains on average 40.59 ng of nucleocapsid.

### Example 21: STX-S and STN-N Individually Induced Strong Immunization, in Absence of Adjuvant

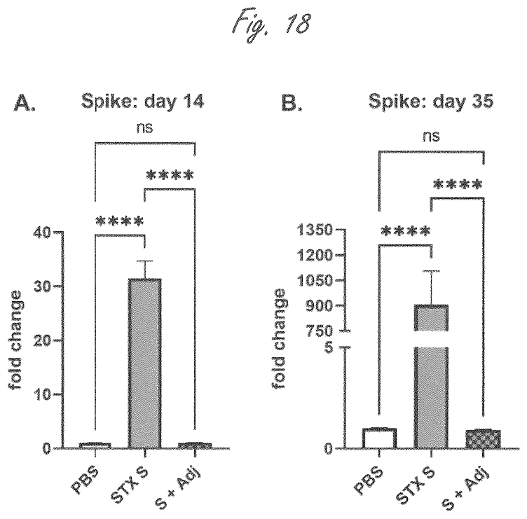

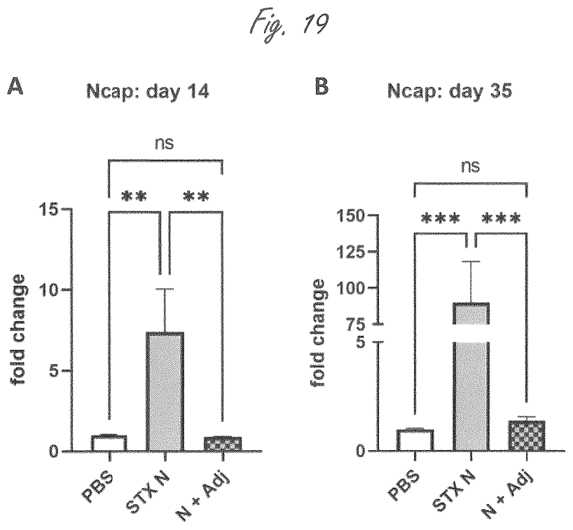

To validate the capability of STX-S and STX-N exosomes to induce an immune-response, spike and nucleocapsid, mice were immunized with 10 ng exosome formulation STX-S and STX-N. As comparison to show the robustness of exosome delivery, 10 ng of either spike or Ncap recombinant protein delivered in conjugation with adjuvant (Alhydrogel, InvivoGen). PBS was used as control. Blood collected 2 weeks after the boost injection (2^{nd} injection) showed that both STX-S and STX-N vaccines increased antibody production respectively against spike and nucleocapsid in all animals. Neither spike or Ncap protein in combination with adjuvant were statistically different than the PBS control (negative control), and no antibody production was observed (FIGS. 18 and 19, respectively).

### Example 22: STX-S+N Vaccine Induces Strong Immunization Against SARS-CoV2 Protein in Mice

The immune response of a multivalent vaccine, obtained by the combination of STX-S and STX-N exosomes, was assessed. STX-S+N was administered at three different doses (Table 2) into mice by two i.m. injections. A second i.m. injection, boost injection, was delivered after a 3-week interval. PBS was used as negative controls in the study.

TABLE 2
Concentration of Spike and Ncap used in the study.

| | Spike ng/injection | Ncap ng/injection |
|---|---|---|
| Dose 1 | 25 | 2.5 |
| Dose 2 | 10 | 4 |
| Dose 3 | 3 | 9 |

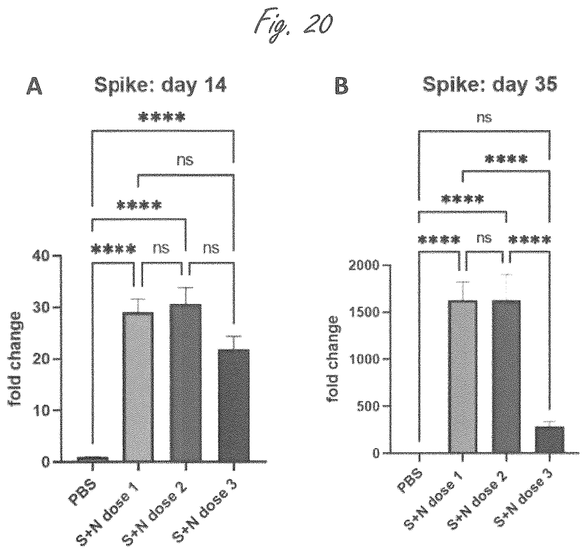

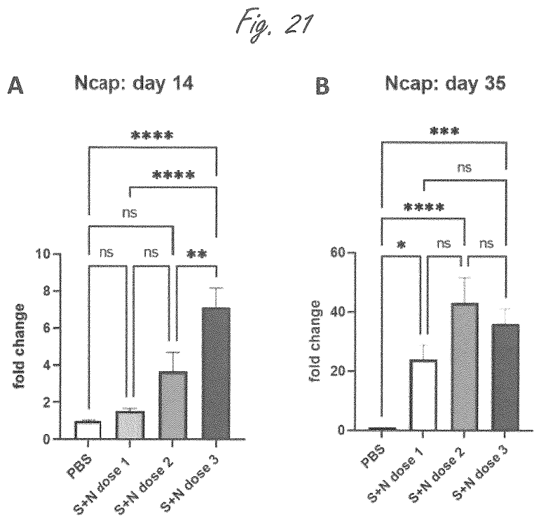

Immunization to the STX-S+N vaccine was evaluated by the quantification of antibodies against Ncap and Spike Ncap (FIG. 20A-B and FIG. 21A-B, respectively). Increase of antibody production was detected after the first injection and continued to increase after the boost injection (2^{nd} injection). A single injection of STX-S+N induced an increase up to 30-fold in IgG against spike, with overall no significant difference between the three dose. After complete immunization, dose 1 (25 ng/spike) and dose 2 (10 ng/spike) resulted in a 1500-fold increase in antibody against Spike, while dose 3 (3 ng/spike, significantly lower amount of spike) resulted in a 280-fold increase. On the other hand, a dose response was observed for Ncap. An increase of 1.5-fold was observed for the low dose 1 (2.5 ng/Ncap), ˜4-fold increase for dose 2 (4 ng/Ncap) and up to 7-fold for dose 3 (9 ng/Ncap). After complete immunization cycle (2 i.m. injections) no significant difference was observed across doses, with an increase in IgG against Neap between 24-43-fold increase observed in STX-S+N treated mice.

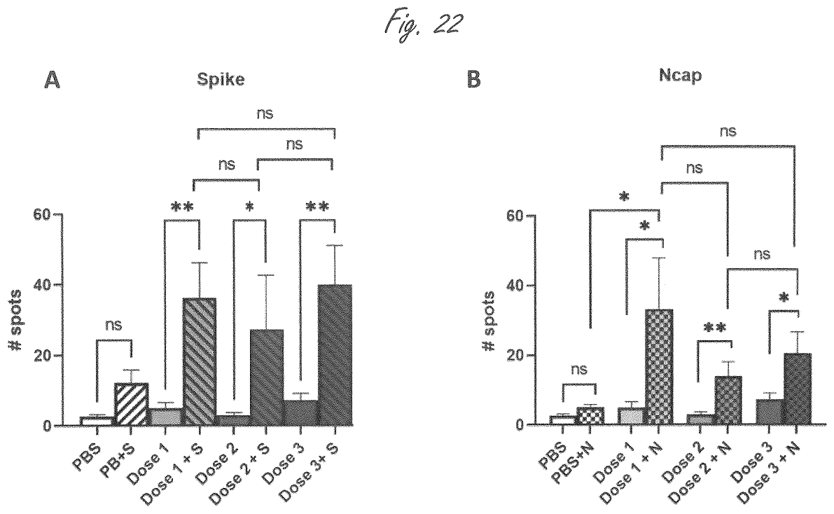

To characterize the T cell response to STX-S+N, antigen-specific T cell responses were measured by ELISpot (FIG. 22). Vaccination with STX-S+N elicited multi-functional, antigen-specific T cell responses. Splenocytes were isolated from animals at day 35 (2 weeks after boost (2^{nd}) injection) and evaluated using ELISpot plates precoated with IFNγ. PBS was used as controls in the study as described above. Baseline expression was compared to stimulation with 10 μg/ml of either Spike or Ncap protein (AcroBiosystem). While baseline IFNγ response was comparable between groups, evaluation of IFNγ secreting cells in response to ex vivo of either Spike (FIG. 22A) or Ncap (FIG. 22B) stimulation showed a strong increase in spleens immunized with STX-S+N vaccine, suggesting a Th1-biased CD8+ T cell response. After spike stimulation, an average of 7-fold increase in IFNγ response was observed despite the STX-S+N dose administered (FIG. 22A). After Ncap stimulation, a dose response effect was observed with 7-fold increase in mice receiving the lowest Ncap dose (dose 1, 2.5 ng) and ˜3-fold increase in mice receiving either dose 2 (4 ng Ncap) or 3 (9 ng Ncap) (FIG. 22B).

### Example 23: STX-S+N Vaccine Induces Strong Immunization Against SARS-CoV2 Protein in Rabbits

Immune response to STX-S+N vaccine, using a clinically relevant dose, was evaluated in rabbits. STX-S+N combination vaccine was administered at two different doses (Table 3) into rabbits by two i.m. injections. A second i.m. injection, boost injection, was delivered after a 2-week interval. PBS was used as controls in the study.

TABLE 3
Concentration of Spike and Ncap used in the study.

| | Spike ng/injection | Ncap ng/injection |
|---|---|---|
| Dose 1 | 125 | 10 |
| Dose 2 | 50 | 20 |

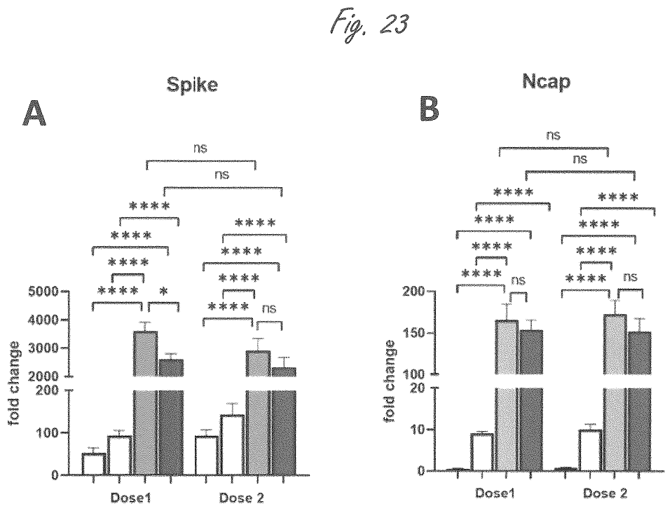

Increased antibody production was detected as early as one week after the first injection and continued to increase after the boost injection (FIG. 7C). After complete immunization cycle (two i.m. injections), up to 3600-fold increase in IgG against Spike (FIG. 23A) and up to 170-fold increase in IgG against Ncap (FIG. 23B) were observed in STX-S+N treated rabbits. No significant differences were observed between doses in antibody production against either antigen.

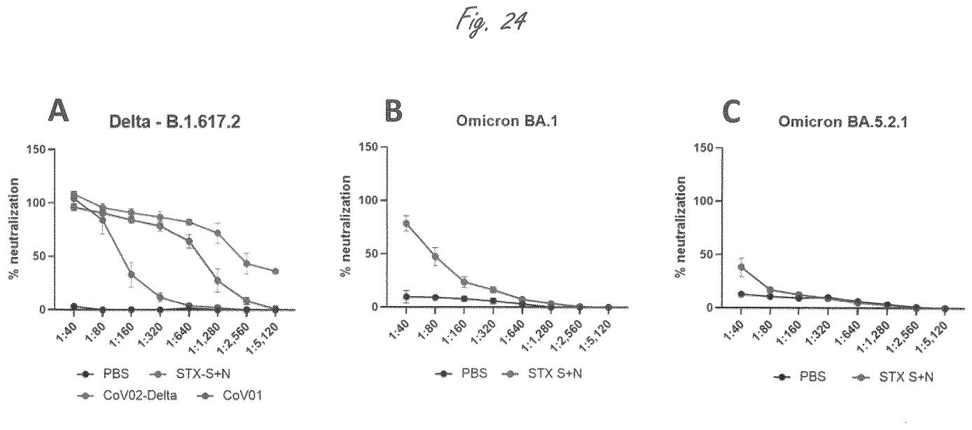

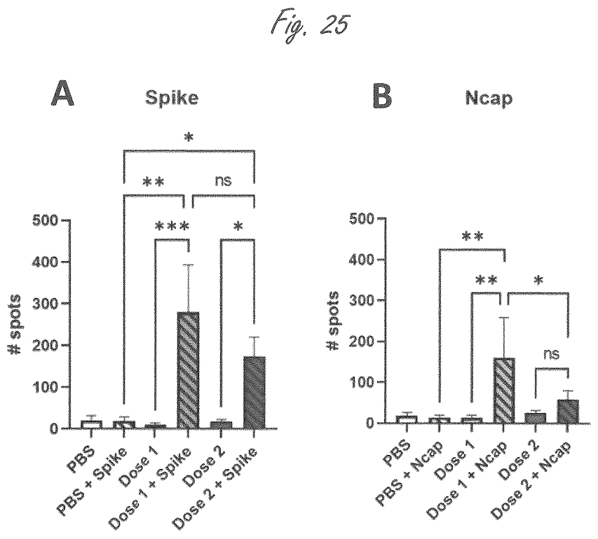

Immunization to the STX-S+N vaccine was further evaluated by assessing neutralizing antibodies against SARS-CoV-2 variants (FIG. 24 A-C). Plasma from rabbits of dose 2 (50 ng S and 20 ng N, 8 animals) and 2 PBS control were tested for neutralizing antibodies against SARS-CoV-2 Delta variant. Potent neutralizing activity was elicited by STX-S+N in all analyzed animals. Importantly, STX-S+N in rabbits induced a response comparable to the human control plasma (COV-02-delta, plasma from a patient fully immunized with Moderna's mRNA vaccine, with breakthrough delta infection), with a complete neutralizing response at higher dilutions (i.e., 1:320, range of 116.74-881.47%; average: 96.22±8.9%) (FIG. 24A). Moreover, STX-S+N in rabbits performed better than CoV-01 control (plasma from a patient who received two doses of the Moderna covid-19 vaccine with no prior history of SARS-CoV-2 infection).

Additionally, the same samples were tested for neutralizing antibodies against SARS-CoV-2 Omicron variants (Omicron BA.1 and BA.5.2.1). As shown in FIGS. 24B and 24C, a strong cross neutralization was observed for the STX-S+N treated rabbits achieving an average neutralization of 75% (range of 40.7-100%) for Omicron BA.1 (FIG. 24B) and a range between 10% to 91% for Omicron BA5 (FIG. 24C). These data suggest that protein-based vaccines delivered by exosomes, specifically STX-S+N, may result in a broader protection against SARS-COV-2 variants. In all assays, rabbits receiving PBS showed no neutralization.

T cell response to STX-S+N immunization was measured by ELISpot (FIGS. 25A and 25B). Vaccination with STX-S+N elicited multi-functional, antigen-specific T cell responses. Splenocytes were isolated from animals at day 28 (2 weeks after boost (2^{nd}) injection) and evaluated using ELISpot plates precoated with IFNg. PBS was used as controls in the study as described above. Baseline expression was compared to stimulation with 10 μg/ml of either Spike or Ncap protein (AcroBiosystem). While baseline IFNγ response was comparable between groups, stimulation with either Spike (FIG. 25A) or Ncap (FIG. 25B) resulted in a strong increase in IFNg production in spleens immunized with STX-S+N vaccine, suggesting a Th1-biased CD8+ T cell response. For spike, a clear dose response was observed with greater response in rabbits receiving dose 1 (125 ng S, +28-fold) than dose 2 (50 ng S, +10-fold), despite not statistically significant (FIG. 25A). For Ncap, dose 1 (10 ng N, +10-fold) resulted in better immune response than dose 2 (20 ng N, +2-fold) (FIG. 25B).

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

What is claimed is:

1. An immunogenic composition comprising an extracellular vesicle and a fusion protein, wherein said fusion protein comprises a SARS-CoV-2 polypeptide and an exosomal polypeptide, and wherein said composition does not comprise an adjuvant.

2. (canceled)

3. The immunogenic composition of claim 1, wherein the fusion protein is present in the composition at a concentration of about 1 ng/100 μL to about 300 ng/100 μL.

4. The immunogenic composition of claim 1, wherein the exosomal polypeptide is a tetraspanin protein polypeptide.

5. The immunogenic composition of claim 1, wherein the SARS-CoV-2 polypeptide is a SARS-CoV-2 spike protein polypeptide.

6. The immunogenic composition of claim 1, wherein the SARS-CoV-2 polypeptide is a SARS-CoV-2 nucleocapsid protein polypeptide.

7. The immunogenic composition of claim 5, wherein the fusion protein comprises an amino acid sequence (i) having at least 80% identity to SEQ ID NO:3, or (ii) of SEQ ID NO:3.

8. The immunogenic composition of claim 6, wherein the fusion protein comprises an amino acid sequence (i) having at least 80% identity to SEQ ID NO:7, or (ii) of SEQ ID NO:7.

9. The immunogenic composition of claim 1, wherein an immunogenic dose of said composition comprises about 1 ng to about 300 ng of fusion protein or virus polypeptide.

10. The immunogenic composition of claim 1 comprising a second vesicle and a second fusion protein, wherein said second fusion protein comprises a second virus polypeptide and an exosomal polypeptide, and wherein said second fusion protein is present in a membrane of the second vesicle.

11. The immunogenic composition of claim 10, wherein said virus polypeptide is a SARS-CoV-2 spike protein polypeptide and said second virus polypeptide is a SARS-CoV-2 nucleocapsid protein.

12. The immunogenic composition of claim 11, wherein the fusion protein comprises an amino acid sequence (i) having at least 80% identity to SEQ ID NO:3 or (ii) of SEQ ID NO:3, and wherein the second fusion protein comprises an amino acid sequence (III) having at least 80% identity to SEQ ID NO:7, or (iv) of SQ ID NO:7.

13. A method of immunizing a subject against a virus infection comprising administering to the subject an immunogenically effective dose of an immunogenic composition comprising an extracellular vesicle and a fusion protein, wherein said fusion protein comprises a SARS-CoV-2 spike protein polypeptide or a SARS-CoV-2 nucleocapsid protein polypeptide; and an exosomal polypeptide, wherein said composition does not comprise an adjuvant.

14. The method of claim 13, wherein said immunogenically effective dose elicits protective immunity in said subject against more than one variant of a given virus.

15. The method of claim 14, wherein the immunogenic composition comprises
a plurality of exosomes;
wherein the nucleocapsid protein polypeptide and/or the spike protein polypeptide is/are present on an outer surface of an exosome of said plurality of exosomes.

16. The method of claim 15, wherein the first fusion protein comprises an amino acid sequence that is (i) at least 80% identical to SEQ ID NO:3 or (ii) set forth in SEQ ID NO:3; and the second fusion protein comprises an amino acid sequence that is (i) at least 80% identical to SEQ ID NO:7 or (ii) set forth in SEQ ID NO:7.

17. The method of claim 15, wherein the immunogenic composition comprises a first fusion protein comprising a SARS-CoV-2 spike protein polypeptide located in the membrane of a first exosome and a second fusion protein comprising a SARS-CoV-2 nucleocapsid protein polypeptide located in the membrane of a second exosome.

18. The method of claim 17, wherein the first fusion protein comprises an amino acid sequence that is (i) at least 80% identical to SEQ ID NO:3 or (ii) set forth in SEQ ID NO:3; and the second fusion protein comprises an amino acid sequence that is (i) at least 80% identical to SEQ ID NO:7 or (ii) set forth in SEQ ID NO:7.

19. The method of claim 15, wherein the immunogenically effective dose comprises about 1 ng to about 300 ng of each of said fusion protein.

20. (canceled)

21. A synthetic fusion protein comprising a SARS-CoV-2 spike protein polypeptide or a SARS-CoV-2 nucleocapsid protein polypeptide, an exosomal polypeptide, and a linker polypeptide.

22.-25. (canceled)

26. The synthetic fusion protein of claim 21, wherein the fusion protein comprises:
(i) an amino acid sequence that is at least 80% identical to SEQ ID NO:3;
(ii) an amino acid sequence of SEQ ID NO:3;
(iii) an amino acid sequence that is at least 80% identical to SEQ ID NO:7; or
(iv) an amino acid sequence of SEQ ID NO:7.

27.-36. (canceled)


SEQUENCE LISTING (12 sequences)

SEQ ID NO: 1 - protein, 1254 aa, SARS-CoV-2
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRGSAGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKF

SEQ ID NO: 2 - protein, 168 aa, synthetic construct
NESLIDLQELGKYEQYIKWPWYIWLGFIAGLIAIVMVTIMLCCMTSCCSCLKGCCSCGSCCKFGGGGSGGGGSGGGGSPVKGGTKCIKYLLFGFNFIFWLAGIAVLAIGLWLRFDSQTKSIFEQETNNNNSSFYTGVYILIGAGALMMLVGFLGCCGAVQESQCMLGL

SEQ ID NO: 3 - protein, 1496 aa, synthetic construct
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRGSAGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFGGGGSGGGGSGGGGSPVKGGTKCIKYLLFGFNFIFWLAGIAVLAIGLWLRFDSQTKSIFEQETNNNNSSFYTGVYILIGAGALMMLVGFLGCCGAVQESQCMLGLFFGFLLVIFAIEIAAAIWGYSHKDEVIKEVQEFYKDTYNKLKTKDEPQRETLKAIHYALNCCGLAGGVEQFISDICPKKDVLETFTVKSCPDAIKEVFDNKFHIIGAVGIGIAVVMIFGMIFSMILCCAIRRNREMV

SEQ ID NO: 4 - DNA, 4488 bp, synthetic construct
  source 1..4488: mol_type = other DNA
atgtttgtatttctggtacttttgccactcgtcagttcccaatgtgttaacctgcgcactcgtacgcaactccccccagcatataccaactcatttacgcggggagtatattatccggacaaggtatttaggagttctgtactgcacagcacacaggacctttttcttccttttttttccaacgtgacatggtttcatgcgatacacgtaagtggaaccaatggaactaaaaggttcgataatcctgtacttccgtttaacgatggtgtctatttcgcttctacggagaagtcaaatatcattagaggttggatcttcggtacaactcttgacagtaagacgcaaagtctgctcatcgtaaacaacgcgacgaacgtagtgataaaggtctgcgagtttcagttctgtaatgatccgtttcttgacgtctattaccataagaacaataagagttggatggaaagtggagtatacagcagcgctaacaattgcacatttgaatatgtctctcaacctttcctgatggacctcgaagggaaacaaggtaacttcaaaaatctccgagaatttgtttttaaaaatatagacggatatttcaagatatactcaaagcacacgccgataaatctggtcagggatctccctcagggtttcagtgcactcgaacctttggtagatttgcctatcggaataaatattacacgctttcagacgcttcttgcgttgcaccggtcttaccttacacctggggacagttcatctggttggacggccggggctgccgcttattacgtcggatatctccaaccaagaacgtttctgcttaaatataatgagaacggcacaataactgacgcggtggattgcgctcttgatcctctctcagagaccaaatgcacgctcaaatcctttactgtcgagaaagggatttatcagaccagcaactttcgcgtgcagcctacagaaagtattgtaagattccctaacattactaacctctgcccgttcggcgaggtatttaatgcaacacgatttgcaagtgtctatgcgtggaatagaaaacgcatttcaaattgtgtcgccgattactccgtactctataacagcgcctcatttagcacgtttaaatgttatggtgtctcccccacaaagctcaatgatctctgcttcactaacgtgtatgctgatagcttcgtgatccgaggtgacgaagtgaggcaaattgctccgggtcaaaccgggaagatcgcagattataactataagttgccagacgatttcactggatgtgttattgcgtggaactctaataacctggactcaaaagtgggagggaattacaattaccgctacaggctgtttcgcaaaagcaatttgaaacccttcgagagggacatcagtactgagatataccaggcagggtcaaaaccctgcaatggagtcgaaggatttaattgctacttcccgcttcagagctatgggttccagccaacgaacggagtgggctaccaaccctatcgggtcgtagttttgagttttgagctgctccacgcccctgcgacggtttgtggacctaagaagagcactaacctcgtgaaaaataagtgtgtcaattttaattttaatggcctgacagggacaggggtccttacagagtccaacaaaaagtttcttccttttcagcagtttgggagggacatagccgatacaaccgacgccgtccgagatccacaaaccttggaaatattggatatcaccccgtgtagtttcggcggagtcagcgttattacccctgggaccaataccagtaaccaggtagccgtattgtatcaaggagttaattgtactgaagtgcctgtggctatccacgcagaccaactgaccccgacatggcgagtttacagtacgggttctaacgtgtttcaaaccagggctggatgtttgatcggtgcagaacacgttaataacagttacgagtgcgatatcccgatcggtgcgggcatttgtgccagttaccaaacacagacaaacagtagaggtagtgctggtagcgttgcgagtcagtctattatagcttacacgatgtcactgggtgctgaaaacagcgtagcctattctaataattccatcgccatacctacaaacttcaccatatctgtgacgactgaaatcctgccagtaagcatgactaagacatcagtggactgtactatgtatatctgtggcgactccacagagtgtagtaatctgttgctgcaatacgggtctttctgtacccagttgaatcgcgcattgacgggcatagccgttgaacaggacaagaacacgcaagaagtattcgcacaagttaaacaaatctataagactccgcctataaaagactttggtggattcaattttagtcaaatacttcctgatccgtccaagccgagtaaacgaagtttcatagaggatttgttgttcaataaggtgacattggctgacgcaggctttatcaaacagtacggtgactgtctcggagacattgctgcgcgagaccttatatgtgcgcaaaagtttaacggacttactgttttgccgcccttgttgacggacgaaatgatagcacaatatacttccgctctgcttgctggaacaattacttctggctggactttcggagcgggtgctgccctccagataccctttgcgatgcaaatggcttacagattcaacggcatcggagtgactcagaacgtcctgtacgaaaaccaaaagcttatcgcaaatcagtttaactctgcaatcggaaaaatccaagacagcttgtctagtacggctagcgcgcttggaaaacttcagaatgttgttaaccagaacgcgcaagcactcaatacgttggttaagcagttgtcatctaatttcggcgcaatctcttcagtacttaacgatattttgtcaaggctcgacccgccggaagccgaagtgcagatcgatagactcataactgggagattgcagtcccttcaaacctatgttacacagcagttgatacgagcggccgagatcagggcgtctgcgaaccttgccgcaacaaagatgtctgagtgcgttttggggcagtccaaacgcgtggatttctgcggtaaagggtatcaccttatgagttttcctcaatcagcgccccacggggtggttttcctccatgtaacgtatgtacccgcacaagaaaagaacttcaccacagctcccgcaatatgccacgacgggaaggcgcacttcccgagggaaggtgttttcgttagtaacggcacgcattggtttgtgacccagagaaatttttacgagccacagatcatcacaacggacaacacattcgtaagtggaaactgtgatgtagtaattggcatcgtcaacaataccgtctacgaccctctccaaccagaactcgattcttttaaggaggaacttgataaatattttaaaaatcatacaagccccgatgtggacctcggagacatcagcggcattaatgcgagcgtggtcaacatccaaaaggagatagatcgcctcaacgaagttgccaagaatttgaatgagagccttatagatcttcaggaactcggcaagtacgagcaatacattaaatggccttggtacatatggctgggatttattgcagggctgatagccatcgtgatggtgacaataatgctctgttgtatgactagttgttgcagctgccttaaagggtgctgttcttgtgggagttgctgtaagtttggcggaggcggaagcggaggcggaggctccggcggaggcggaagcccggtcaaaggaggcaccaagtgcatcaaatacctgctgttcggatttaacttcatcttctggcttgccgggattgctgtccttgccattggactatggctccgattcgactctcagaccaagagcatcttcgagcaagaaactaataataataattccagcttctacacaggagtctatattctgatcggagccggcgccctcatgatgctggtgggcttcctgggctgctgcggggctgtgcaggagtcccagtgcatgctgggactgttcttcggcttcctcttggtgatattcgccattgaaatagctgcggccatctggggatattcccacaaggatgaggtgattaaggaagtccaggagttttacaaggacacctacaacaagctgaaaaccaaggatgagccccagcgggaaacgctgaaagccatccactatgcgttgaactgctgtggtttggctgggggcgtggaacagtttatctcagacatctgccccaagaaggacgtactcgaaaccttcaccgtgaagtcctgtcctgatgccatcaaagaggtcttcgacaataaattccacatcatcggcgcagtgggcatcggcattgccgtggtcatgatatttggcatgatcttcagtatgatcttgtgctgtgctatccgcaggaaccgcgagatggtc

SEQ ID NO: 5 - protein, 418 aa, SARS-CoV-2
SDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 6 - protein, 240 aa, synthetic construct
WLTYTGAIKLDDKDPNFKDQVILLNKHIDAYKTFPPTEPKKDKKKKADETQALPQRQKKQQTVTLLPAADLDDFSKQLQQSMSSADSTQAKPTTTPAPRPPTPAPTIASQPLSLRPEACRPAAGGAVHTRGLDFACDIYIWAPLAGTCGVLLLSLVITLYCNHRNGGGGSGGGGSGGGGSPVKGGTKCIKYLLFGFNFIFWLAGIAVLAIGLWLRFDSQTKSIFEQETNNNNSSFYTGVY

SEQ ID NO: 7 - protein, 757 aa, synthetic construct
MLLLVTSLLLCELPHPAFLLIPSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQAKPTTTPAPRPPTPAPTIASQPLSLRPEACRPAAGGAVHTRGLDFACDIYIWAPLAGTCGVLLLSLVITLYCNHRNGGGGSGGGGSGGGGSPVKGGTKCIKYLLFGFNFIFWLAGIAVLAIGLWLRFDSQTKSIFEQETNNNNSSFYTGVYILIGAGALMMLVGFLGCCGAVQESQCMLGLFFGFLLVIFAIEIAAAIWGYSHKDEVIKEVQEFYKDTYNKLKTKDEPQRETLKAIHYALNCCGLAGGVEQFISDICPKKDVLETFTVKSCPDAIKEVFDNKFHIIGAVGIGIAVVMIFGMIFSMILCCAIRRNREMV

SEQ ID NO: 8 - DNA, 2286 bp, synthetic construct
  source 1..2286: mol_type = other DNA
tctagagccaccatgctgctgctggtgaccagcctgctgctgtgtgagctgccccaccccgcctttctgctgatcccctctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggccaagcccaccaccacccctgcccctagacctccaaccccagcccctacaatcgccagccagcccctgagcctgaggcccgaagcctgtagacctgccgctggcggagccgtgcacaccagaggcctggatttcgcctgcgacatctacatctgggcccctctggccggcacctgtggcgtgctgctgctgagcctggtcatcaccctgtactgcaaccaccggaatggcggaggcggaagcggaggcggaggctccggcggaggcggaagcccggtcaaaggaggcaccaagtgcatcaaatacctgctgttcggatttaacttcatcttctggcttgccgggattgctgtccttgccattggactatggctccgattcgactctcagaccaagagcatcttcgagcaagaaactaataataataattccagcttctacacaggagtctatattctgatcggagccggcgccctcatgatgctggtgggcttcctgggctgctgcggggctgtgcaggagtcccagtgcatgctgggactgttcttcggcttcctcttggtgatattcgccattgaaatagctgcggccatctggggatattcccacaaggatgaggtgattaaggaagtccaggagttttacaaggacacctacaacaagctgaaaaccaaggatgagccccagcgggaaacgctgaaagccatccactatgcgttgaactgctgtggtttggctgggggcgtggaacagtttatctcagacatctgccccaagaaggacgtactcgaaaccttcaccgtgaagtcctgtcctgatgccatcaaagaggtcttcgacaataaattccacatcatcggcgcagtgggcatcggcattgccgtggtcatgatatttggcatgatcttcagtatgatcttgtgctgtgctatccgcaggaaccgcgagatggtctaa

SEQ ID NO: 9 - protein, 28 aa, Homo sapiens
IYIWAPLAGTCGVLLLSLVITLYCNHRN

SEQ ID NO: 10 - protein, 227 aa, Homo sapiens
PVKGGTKCIKYLLFGFNFIFWLAGIAVLAIGLWLRFDSQTKSIFEQETNNNNSSFYTGVYILIGAGALMMLVGFLGCCGAVQESQCMLGLFFGFLLVIFAIEIAAAIWGYSHKDEVIKEVQEFYKDTYNKLKTKDEPQRETLKAIHYALNCCGLAGGVEQFISDICPKKDVLETFTVKSCPDAIKEVFDNKFHIIGAVGIGIAVVMIFGMIFSMILCCAIRRNREMV

SEQ ID NO: 11 - DNA, 3762 bp, SARS-CoV-2
  source 1..3762: mol_type = other DNA
atgtttgtatttctggtacttttgccactcgtcagttcccaatgtgttaacctgcgcactcgtacgcaactccccccagcatataccaactcatttacgcggggagtatattatccggacaaggtatttaggagttctgtactgcacagcacacaggacctttttcttccttttttttccaacgtgacatggtttcatgcgatacacgtaagtggaaccaatggaactaaaaggttcgataatcctgtacttccgtttaacgatggtgtctatttcgcttctacggagaagtcaaatatcattagaggttggatcttcggtacaactcttgacagtaagacgcaaagtctgctcatcgtaaacaacgcgacgaacgtagtgataaaggtctgcgagtttcagttctgtaatgatccgtttcttgacgtctattaccataagaacaataagagttggatggaaagtggagtatacagcagcgctaacaattgcacatttgaatatgtctctcaacctttcctgatggacctcgaagggaaacaaggtaacttcaaaaatctccgagaatttgtttttaaaaatatagacggatatttcaagatatactcaaagcacacgccgataaatctggtcagggatctccctcagggtttcagtgcactcgaacctttggtagatttgcctatcggaataaatattacacgctttcagacgcttcttgcgttgcaccggtcttaccttacacctggggacagttcatctggttggacggccggggctgccgcttattacgtcggatatctccaaccaagaacgtttctgcttaaatataatgagaacggcacaataactgacgcggtggattgcgctcttgatcctctctcagagaccaaatgcacgctcaaatcctttactgtcgagaaagggatttatcagaccagcaactttcgcgtgcagcctacagaaagtattgtaagattccctaacattactaacctctgcccgttcggcgaggtatttaatgcaacacgatttgcaagtgtctatgcgtggaatagaaaacgcatttcaaattgtgtcgccgattactccgtactctataacagcgcctcatttagcacgtttaaatgttatggtgtctcccccacaaagctcaatgatctctgcttcactaacgtgtatgctgatagcttcgtgatccgaggtgacgaagtgaggcaaattgctccgggtcaaaccgggaagatcgcagattataactataagttgccagacgatttcactggatgtgttattgcgtggaactctaataacctggactcaaaagtgggagggaattacaattaccgctacaggctgtttcgcaaaagcaatttgaaacccttcgagagggacatcagtactgagatataccaggcagggtcaaaaccctgcaatggagtcgaaggatttaattgctacttcccgcttcagagctatgggttccagccaacgaacggagtgggctaccaaccctatcgggtcgtagttttgagttttgagctgctccacgcccctgcgacggtttgtggacctaagaagagcactaacctcgtgaaaaataagtgtgtcaattttaattttaatggcctgacagggacaggggtccttacagagtccaacaaaaagtttcttccttttcagcagtttgggagggacatagccgatacaaccgacgccgtccgagatccacaaaccttggaaatattggatatcaccccgtgtagtttcggcggagtcagcgttattacccctgggaccaataccagtaaccaggtagccgtattgtatcaaggagttaattgtactgaagtgcctgtggctatccacgcagaccaactgaccccgacatggcgagtttacagtacgggttctaacgtgtttcaaaccagggctggatgtttgatcggtgcagaacacgttaataacagttacgagtgcgatatcccgatcggtgcgggcatttgtgccagttaccaaacacagacaaacagtagaggtagtgctggtagcgttgcgagtcagtctattatagcttacacgatgtcactgggtgctgaaaacagcgtagcctattctaataattccatcgccatacctacaaacttcaccatatctgtgacgactgaaatcctgccagtaagcatgactaagacatcagtggactgtactatgtatatctgtggcgactccacagagtgtagtaatctgttgctgcaatacgggtctttctgtacccagttgaatcgcgcattgacgggcatagccgttgaacaggacaagaacacgcaagaagtattcgcacaagttaaacaaatctataagactccgcctataaaagactttggtggattcaattttagtcaaatacttcctgatccgtccaagccgagtaaacgaagtttcatagaggatttgttgttcaataaggtgacattggctgacgcaggctttatcaaacagtacggtgactgtctcggagacattgctgcgcgagaccttatatgtgcgcaaaagtttaacggacttactgttttgccgcccttgttgacggacgaaatgatagcacaatatacttccgctctgcttgctggaacaattacttctggctggactttcggagcgggtgctgccctccagataccctttgcgatgcaaatggcttacagattcaacggcatcggagtgactcagaacgtcctgtacgaaaaccaaaagcttatcgcaaatcagtttaactctgcaatcggaaaaatccaagacagcttgtctagtacggctagcgcgcttggaaaacttcagaatgttgttaaccagaacgcgcaagcactcaatacgttggttaagcagttgtcatctaatttcggcgcaatctcttcagtacttaacgatattttgtcaaggctcgacccgccggaagccgaagtgcagatcgatagactcataactgggagattgcagtcccttcaaacctatgttacacagcagttgatacgagcggccgagatcagggcgtctgcgaaccttgccgcaacaaagatgtctgagtgcgttttggggcagtccaaacgcgtggatttctgcggtaaagggtatcaccttatgagttttcctcaatcagcgccccacggggtggttttcctccatgtaacgtatgtacccgcacaagaaaagaacttcaccacagctcccgcaatatgccacgacgggaaggcgcacttcccgagggaaggtgttttcgttagtaacggcacgcattggtttgtgacccagagaaatttttacgagccacagatcatcacaacggacaacacattcgtaagtggaaactgtgatgtagtaattggcatcgtcaacaataccgtctacgaccctctccaaccagaactcgattcttttaaggaggaacttgataaatattttaaaaatcatacaagccccgatgtggacctcggagacatcagcggcattaatgcgagcgtggtcaacatccaaaaggagatagatcgcctcaacgaagttgccaagaatttgaatgagagccttatagatcttcaggaactcggcaagtacgagcaatacattaaatggccttggtacatatggctgggatttattgcagggctgatagccatcgtgatggtgacaataatgctctgttgtatgactagttgttgcagctgccttaaagggtgctgttcttgtgggagttgctgtaagttt

SEQ ID NO: 12 - DNA, 1254 bp, SARS-CoV-2
  source 1..1254: mol_type = other DNA
tctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcc